(19) United States
(12) Patent Application Publication
(10) Pub. No.: US 2011/0142952 A1
(43) Pub. Date: Jun. 16, 2011

(54) Pharmaceutical Compositions

(76) Inventors: David J. Harris, Lexington, MA (US); Stephen Randall Holmes-Farley, Arlington, MA (US); Steven C. Polomoscanlk, Methuen, MA (US); Adnan Salameh, Irvine, CA (US); Bruce Shutts, Bolton, MA (US); Richard Silva, Needham, MA (US)

(21) Appl. No.: 13/000,114

(22) PCT Filed: Jun. 17, 2009
(86) PCT No.: PCT/US09/03615

§ 371 Date: Feb. 9, 2011

Related U.S. Application Data
(60) Provisional application No. 61/129,360, filed on Jun. 20, 2008.

(51) Int. Cl. A61K 9/16; A61K 31/785; A61K 31/132; C08G 73/02; C07C 217/42; B32B 5/16

(52) U.S. Cl. 424/501; 424/78.08; 514/668; 424/489; 528/422; 564/504; 428/402

(57) ABSTRACT

This invention relates to crosslinked amine-containing polymers for binding compounds or ions, and more specifically relates to pharmaceutically acceptable compositions for binding compounds or ions that include crosslinked amine-containing polymers. The pharmaceutically acceptable composition includes, for example, crosslinked polyamine particles, or pharmaceutically acceptable salts thereof, having a particle size distribution wherein greater than 10 vol. % of the particles have a particle size greater than 500 μm.

### FIELD OF THE INVENTION

This invention relates to crosslinked amine-containing polymers for binding compounds or ions, and more specifically relates to pharmaceutically acceptable compositions for binding compounds or ions that include crosslinked amine-containing polymers.

### BACKGROUND OF THE INVENTION

Hyperphosphatemia frequently accompanies diseases associated with inadequate renal function such as end stage renal disease (ESRD), hyperparathyroidism, and certain other medical conditions. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of insoluble phosphate binders to reduce gastrointestinal absorption. Many such treatments have a variety of unwanted side effects and/or have less than optimal phosphate binding properties, including potency and efficacy. Accordingly, there is a need for compositions and treatments with good phosphate-binding properties and good side effect profiles.

### DEFINITIONS

The following definitions apply herein unless otherwise specifically noted:

Aggregate particle: an aggregate particle is a particle that is assembled from, formed from or comprises distinct constituent particles.

d₁₀: the particle size within a distribution of particles where 10 vol. % of the particles have a smaller particle size.

d₅₀: the particle size within a distribution of particles where 50 vol. % of the particles have a particle size that is larger and where 50 vol. % of the particles have a particle size that is smaller.

d₉₀: the particle size within a distribution of particles where 90 vol. % of the particles have a smaller particle size.

Crosslinked polyamine particles: particles comprising at least one crosslinked polyamine, for example particles that comprise at least a substantial portion, by weight, of a crosslinked polyamine, wherein the substantial portion is at least 50 wt. %, 60 wt. %, 70 wt. %, 80 wt. %, 90 wt. %, 95 wt. %, 98 wt. %, or 99 wt. % as well as 100 wt. %.

### BRIEF SUMMARY OF THE INVENTION

In one aspect, the present invention relates to crosslinked polyamine particles and/or pharmaceutical compositions comprising, at least in part, crosslinked polyamine particles. Compositions can comprise one or more crosslinked polyamines. Several embodiments of the invention are described in further detail as follows. Generally, each of these embodiments can be used in various and specific combinations, and with other aspects and embodiments unless otherwise stated herein.

In addition to the crosslinked polyamine particles of the present invention as described herein, other forms of the crosslinked polyamine particles are within the scope of the invention including pharmaceutically acceptable salts, solvates, hydrates, prodrugs, polymorphs, clathrates, and isotopic variants and mixtures thereof of the crosslinked polyamine particles.

In addition, crosslinked polyamine particles of the invention may have optical centers or chiral centers and the crosslinked polyamine particles of the present invention include all of the isomeric forms of these crosslinked polyamine particles, including optically pure forms, racemates, diastereomers, enantiomers, tautomers and/or mixtures thereof.

In some embodiments, the crosslinked polyamine particles may have a particle size distribution such that greater than 90 vol. % of the crosslinked polyamine particles have a particle size between 250 μm and 4 mm. In some embodiments, the crosslinked polyamine particles may have a particle size distribution where greater than 5 vol. % of the crosslinked polyamine particles has a particle size larger than 500 μm. In some embodiments, the crosslinked polyamine particles have a particle size distribution such that no more than 0 to 20 vol. % of the crosslinked polyamine particles has a particle size smaller than 300 μm. In some embodiments, the crosslinked polyamine particles may have a particle size distribution such that the d₁₀ value is between 250 μm and 750 μm and/or the d₉₀ value is between 900 μm and 1600 μm. In some embodiments, the crosslinked polyamine particles may have a d₅₀ that is between 450 μm and 1100 μm.

In some embodiments, 75 wt. % to 100 wt. % of the crosslinked polyamine particles have a mesh size that is −5/+60. In some embodiments, greater than 5 wt. % of the crosslinked polyamine particles have a mesh size that is +35. In some embodiments, no more than 0 to 20 wt. % of the crosslinked polyamine particles have a mesh size that is −50. In some embodiments, between 40 wt. % and 60 wt. % of the crosslinked polyamine particles have a mesh size that is −16/+40.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g. phosphate), from the gastrointestinal tract of an animal by administering an effective amount of crosslinked polyamine particles or a pharmaceutical composition comprising crosslinked polyamine particles wherein the crosslinked polyamine particles has one or more of the particle size characteristics described herein, such as for example, a particle size distribution such that greater than 5 vol. % of the crosslinked polyamine particles have a particle size greater than 500 μm, such as between 500 μm and 2 mm.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g. phosphate), from the gastrointestinal tract of an animal by administering an effective amount of crosslinked polyamine particles or a pharmaceutical composition comprising crosslinked polyamine particles, wherein the crosslinked polyamine particles have a mean gray value of greater than 180.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g. phosphate), from the gastrointestinal tract of an animal by administering an effective amount of a crosslinked polyamine particles or a pharmaceutical composition comprising crosslinked polyamine particles, wherein said crosslinked polyamine particles comprise 2 or more constituent particles comprising crosslinked polyamine.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g. phosphate), from the gastrointestinal tract of an animal by administering an effective amount of a crosslinked polyamine particles or a pharmaceutical composition comprising crosslinked polyamine particles, wherein the crosslinked polyamine particles are formed by aggregating 2 or more constituent particles comprising crosslinked polyamine.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g. phosphate), from the gastrointestinal tract of an animal by administering an effective amount of crosslinked polyamine particles or a pharmaceutical composition comprising crosslinked polyamine particles, wherein the crosslinked polyamine particles have an in vitro competitive phosphate binding capacity of greater than 1.2 mmol/g at 60 minutes.

In some embodiments, crosslinked polyamine particles according to the invention may have one or more of or any combination of the following characteristics:
a) a particle size distribution such that 75 vol. % or greater of the crosslinked polyamine particles have a size of between 250 μm and 4 mm;
b) a particle size distribution where from 5 vol. % to 100 vol. % of the crosslinked polyamine particles have a particle size of greater than 500 μm;
c) a particle size distribution such that no more than 20 vol. % of the crosslinked polyamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180;
l) comprises 2 or more constituent particles; and/or
m) a competitive phosphate binding capacity at 60 minutes of greater than 1.2.

In some embodiments, the crosslinked polyamine particles described herein may comprise aggregates of constituent particles of the crosslinked polyamine polymers. In some embodiments, the constituent particles may have a particle size distribution such that greater than 70% of the constituent particles have a particle size between 50 μm and 850 μm. In some embodiments, the constituent particles may have a particle size distribution such that the constituent particles have a d₁₀ value between about 20 μm and about 100 μm and/or a d₉₀ value that is between about 150 μm and about 450 μm. In some embodiments, the constituent particles may have a d₅₀ between 50 μm and 200 μm. In some embodiments, the crosslinked polyamine particles comprise aggregates of from about 2 to about 10,000 constituent particles.

In some embodiments, the invention provides methods of treating an animal, including a human. The method generally involves administering an effective amount of crosslinked polyamine particles or a composition (e.g., a pharmaceutical composition) comprising the same to the animal as described herein.

Another aspect of the invention is a pharmaceutical composition comprising crosslinked polyamine particles of the present invention and at least one pharmaceutically acceptable excipient. In some embodiments, the composition is a liquid formulation in which the crosslinked polyamine particles are dispersed in a liquid vehicle, such as water, and suitable excipients. In some embodiments, the invention provides a pharmaceutical composition comprising crosslinked polyamine particles for binding a target compound or ion, and one or more suitable pharmaceutical excipients, where the composition is in the form of a tablet, sachet, slurry, food formulation, troche, capsule, elixir, suspension, syrup, wafer, chewing gum or lozenge. In some embodiments the composition contains a pharmaceutical excipient selected from the group consisting of sucrose, mannitol, xylitol, maltodextrin, fructose, sorbitol, and combinations thereof. In some embodiments the target anion of the crosslinked polyamine particles is an organophosphate and/or phosphate. In some embodiments the crosslinked polyamine particles are more than about 50% of the weight of the tablet. In some embodiments, the tablet is of cylindrical shape with a diameter of from about 12 mm to about 28 mm and a height of from about 1 mm to about 8 mm and the crosslinked polyamine particles comprise more than 0.6 to about 2.0 gm of the total weight of the tablet.

In some of the compositions of the invention, the excipients are chosen from the group consisting of sweetening agents, binders, lubricants, and disintegrants. In some of these embodiments, the sweetening agent is selected from the group consisting of sucrose, mannitol, xylitol, maltodextrin, fructose, and sorbitol, and combinations thereof.

The crosslinked polyamine particles described herein have several therapeutic applications. For example, the crosslinked polyamine particles are useful in removing compounds or ions such as anions, for example phosphorous-containing compounds or phosphorous containing ions such as organophosphates and/or phosphates, from the gastrointestinal tract, such as from the stomach, small intestine and/or large intestine. In some embodiments, the crosslinked amine polymers are used in the treatment of phosphate imbalance disorders and renal diseases.

In yet another aspect, the crosslinked polyamine particles are useful for removing other solutes, such as chloride, bicarbonate, and/or oxalate containing compounds or ions. Crosslinked polyamine particles removing oxalate compounds or ions find use in the treatment of oxalate imbalance disorders. Crosslinked polyamine particles removing chloride compounds or ions find use in treating acidosis, for example. In some embodiments, the crosslinked polyamine particles are useful for removing bile acids, citrate and related compounds.

The invention further provides compositions containing any of the above crosslinked polyamine particles where the crosslinked polyamine particles are encased in one or more shells.

### DETAILED DESCRIPTION OF THE INVENTION

In one aspect, the present invention provides crosslinked polyamine particles, compositions and methods of using crosslinked polyamine particles, where the crosslinked polyamine comprises or is derived from an amine compound, polymer or copolymer and a crosslinking agent.

As used herein, unless otherwise stated, the term “derived from” is understood to mean: produced or obtained from another substance by chemical reaction, especially directly derived from the reactants, for example a crosslinked polyamine may be derived from the reaction of an amine compound and a linking agent, such as a crosslinking agent resulting in a crosslinked polyamine that is derived from the amine compound and the crosslinking agent.

In some embodiments, it has been found that the size and/or size distribution of the crosslinked polyamine particles of the invention affect the ion binding, such as the phosphate binding properties of the polymers. In some embodiments, crosslinked polyamine particles of the invention may exhibit enhanced phosphate binding in the presence of competing organic ions throughout a physiologically significant time period while having similar equilibrium phosphate binding properties when compared to smaller particles of the same polymer.

The particle size of the crosslinked polyamine particles may be determined according to the procedure detailed in the Test Procedures. In some embodiments, crosslinked polyamine particles have a particle size distribution such that 75 vol. % or greater, such as 80 vol. % or greater, 85 vol. % or greater, 90 vol. % or greater, 95 vol. % or greater, 99 vol. % or greater, or 100 vol. % of the crosslinked polyamine particles have a particle size between 250 μm and 4 mm, such as between 275 μm and 3.5 mm, between 300 μm and 3.0 mm, between 300 μm and 2.5 mm, between 300 μm and 2.0 mm, between 325 μm and 2.5 mm, between 350 μm and 2.0 mm, between 375 μm and 1.75 mm, between 400 μm and 1500 μm, between 425 μm and 1400 μm, between 450 μm and 1300 μm, between 475 μm and 1200 μm, between 500 μm and 1100 μm, or between 525 μm and 1075 μm.

In some embodiments of the invention, the crosslinked polyamine particles have a particle size distribution such that greater than 5 vol. %, greater than 10 vol. %, greater than 20 vol. %, greater than 30 vol. %, greater than 40 vol. %, greater than 50 vol. %, greater than 60 vol. %, greater than 70 vol. %, greater than 80 vol. %, greater than 90 vol. % or greater than 95 vol. % of the crosslinked polyamine particles have a particle size of greater than 450 μm, such as greater than 500 μm, greater than 525 μm, greater than 550 μm, greater than 575 μm, greater than 600 μm, greater than 625 μm, greater than 650 μm, greater than 675, greater than 700 μm, greater than 725 μm, greater than 750 μm or greater than 775 μm.

In some embodiments of the invention, the crosslinked polyamine particles have a particle size distribution such that greater than 5 vol. %, greater than 10 vol. %, greater than 20 vol. %, greater than 30 vol. %, greater than 40 vol. %, greater than 50 vol. %, greater than 60 vol. %, greater than 70 vol. %, greater than 80 vol. %, greater than 90 vol. % or greater than 95 vol. % of the crosslinked polyamine particles have a particle size of between 500 μm and 2.0 mm, such as between 525 μm and 1800 μm, between 550 μm and 1600 μm, between 575 μm and 1550 μm, between 600 μm and 1500 μm, between 625 μm and 1475 μm, between 650 μm and 1450 μm, between 675 μm and 1425 μm, between 700 μm and 1400 μm, between 725 μm and 1375 μm, between 750 μm and 1350 μm or between 775 μm and 1300 μm.

In some embodiments of the invention, the crosslinked polyamine particles have a particle size distribution such that from 5 to 100 vol. %, 10 to 90 vol. %, 20 to 80 vol. %, 30 to 70 vol. %, 40 to 60 vol. % or 50 vol. % of the crosslinked polyamine particles have a particle size of greater than 450 μm, such as greater than 500 μm, greater than 525 μm, greater than 550 μm, greater than 575 μm, greater than 600 μm, greater than 625 μm, greater than 650 μm, greater than 675 μm, greater than 700 μm, greater than 725 μm, greater than 750 μm or greater than 775 μm.

In some embodiments of the invention, the crosslinked polyamine particles have a particle size distribution such that from 5 to 100 vol. %, 10 to 90 vol. %, 20 to 80 vol. %, 30 to 70 vol. %, 40 to 60 vol. % or 50 vol. % of the crosslinked polyamine particles have a particle size of between 500 μm and 2.0 mm, such as between 525 μm and 1800 μm, between 550 μm and 1600 μm, between 575 μm and 1550 μm, between 600 μm and 1500 μm, between 625 μm and 1475 μm, between 650 μm and 1450 μm, between 675 μm and 1425 μm, between 700 μm and 1400 μm, between 725 μm and 1375 μm, between 750 μm and 1350 μm or between 775 μm and 1300 μm.

In some embodiments of the invention, the crosslinked polyamine particles have a particle size distribution such that no more than 0 to 20 vol. %, such as no more than 5 to 15 vol. %, such as no more than 5 vol. %, 10 vol. %, 15 vol. % or 20 vol. % of the crosslinked polyamine particles have a particle size of less than about 300 μm. In some embodiments of the invention, the crosslinked polyamine particles have a particle size distribution such that no more than 0 to 25 vol. %, such as no more than 5 to 20 vol. %, such as no more than 5 vol. %, 10 vol. %, 15 vol. %, 20 vol. % or no more than 25 vol. % of the crosslinked polyamine particles have a particle size of less than about 350 μm. In some embodiments of the invention, the crosslinked polyamine particles have a particle size distribution such that no more than 0 to 35 vol. %, such as no more than 5 to 30 vol. %, such as no more than 10 vol. %, 15 vol. %, 20 vol. %, 25 vol. % or no more than 30 vol. % of the crosslinked polyamine particles have a particle size of less than about 400 μm. In some embodiments of the invention, the crosslinked polyamine particles have a particle size distribution such that no more than 0 to 40 vol. %, such as no more than 5 to 35 vol. %, such as no more than 10 vol. %, 15 vol. %, 20 vol. %, 25 vol. %, 20 vol. %, 35 vol. % or no more than 40 vol. % of the crosslinked polyamine particles has a particle size of less than about 450 μm.

In some embodiments of the invention, the crosslinked polyamine particles have a particle size distribution such that d₁₀ is greater than 225 μm, such as greater than 250 μm, greater than 275 μm, greater than 300 μm, greater than 325 μm greater than 350 μm, greater than 375 μm, greater than 400 μm, greater than 425, μm, greater than 450 μm, greater than 475 μm, greater than 500 μm, greater than 525 μm, or greater than 550 μm.

In some embodiments of the invention, the crosslinked polyamine particles have a particle size distribution such that d₁₀ is between 275 μm and 725 μm, between 300 μm and 700 μm, between 325 μm and 675 μm, between 350 μm and 650 μm, between 375 μm and 625 μm.

In some embodiments of the invention, the crosslinked polyamine particles have a particle size distribution such that d₉₀ is less than 1650 μm, such as less than 1600 μm, less than 1550 μm, less than 1500 μm, less than 1475 μm, less than 1450 μm, less than 1425 μm, less than 1400 μm, less than 1350 μm, less than 1300 μm.

In some embodiments of the invention, the crosslinked polyamine particles have a particle size distribution such that d₉₀ is between 900 μm and 1600 μm, such as between 925 μm and 1550 μm, between 950 μm and 1525 μm, between 975 μm and 1500 μm, between 1000 μm and 1475 μm, between 1025 μm and 1450 μm, between 1050 μm and 1425 μm, between 1075 μm and 1400 μm, between 1100 μm and 1400 μm, between 1100 μm and 1375 μm, between 1100 μm and 1350 μm or between 1100 μm and 1325 μm.

In some embodiments of the invention, the crosslinked polyamine particles have a particle size distribution such that d₁₀ is greater than 225 μm, such as greater than 250 μm, greater than 275 μm, greater than 300 μm, greater than 325 μm, greater than 350 μm, greater than 375 μm, greater than 400 μm, greater than 425, μm, greater than 450 μm, greater than 475 μm, greater than 500 μm, greater than 525 μm, or greater than 550 μm and d₉₀ is less than 1650 μm, such as less than 1600 μm, less than 1550 μm, less than 1500 μm, less than 1475 μm, less than 1450 μm, less than 1425 μm, less than 1400 μm, less than 1350 μm, less than 1300 μm.

In some embodiments of the invention, the crosslinked polyamine particles have a particle size distribution such that d₁₀ is greater than 225 μm, such as greater than 250 μm, greater than 275 μm, greater than 300 μm, greater than 325 μm, greater than 350 μm, greater than 375 μm, greater than 400 μm, greater than 425, μm, greater than 450 μm, greater than 475 μm, greater than 500 μm, greater than 525 μm, or greater than 550 μm and d₉₀ is between 900 μm and 1600 μm, such as between 925 μm and 1550 μm, between 950 μm and 1525 μm, between 975 μm and 1500 μm, between 1000 μm and 1475 μm, between 1025 μm and 1450 μm, between 1050 μm and 1425 μm, between 1075 μm and 1400 μm, between 1100 μm and 1400 μm, between 1100 μm and 1375 μm, between 1100 μm and 1350 μm or between 1100 μm and 1325 μm.

In some embodiments of the invention, the crosslinked polyamine particles have a particle size distribution such that d₁₀ is between 275 μm and 725 μm, between 300 μm and 700 μm, between 325 μm and 675 μm, between 350 μm and 650 μm, between 375 μm and 625 μm and d₉₀ is less than 1650 μm, such as less than 1600 μm, less than 1550 μm, less than 1500 μm, less than 1475 μm, less than 1450 μm, less than 1425 μm, less than 1400 μm, less than 1350 μm, less than 1300 μm.

In some embodiments of the invention, the crosslinked polyamine particles have a particle size distribution such that d₁₀ is between 275 μm and 725 μm, between 300 μm and 700 μm, between 325 μm and 675 μm, between 350 μm and 650 μm, between 375 μm and 625 μm and d₉₀ is between 900 μm and 1600 μm, such as between 925 μm and 1550 μm, between 950 μm and 1525 μm, between 975 μm and 1500 μm, between 1000 μm and 1475 μm, between 1025 μm and 1450 μm, between 1050 μm and 1425 μm, between 1075 μm and 1400 μm, between 1100 μm and 1400 μm, between 1100 μm and 1375 μm, between 1100 μm and 1350 μm or between 1100 μm and 1325 μm.

In some embodiments of the invention, the crosslinked polyamine particles have a d₅₀ that is greater than 450 μm, such as greater than 475 μm, greater than 500 μm, greater than 525 μm, greater than 550 μm, greater than 575 μm, greater than 600 μm, greater than 625 μm, greater than 650 μm, greater than 675 μm or greater than 700 μm.

In some embodiments of the invention, the crosslinked polyamine particles have a d₅₀ between 450 μm and 1100 μm, such as between 475 μm and 1050 μm, between 500 μm and 1025 μm, between 525 μm and 1000 μm, between 550 μm and 975 μm, between 575 μm and 950 μm, between 600 μm and 925 μm, between 625 μm and 900 μm, between 650 μm and 875 μm, between 675 μm and 850 μm or between 700 μm and 825 μm. In some embodiments, the crosslinked polyamine particles have a d₅₀ between 675 μm and 1000 μm.

In some embodiments, crosslinked polyamine particles of the invention may be sized according to sieve size with a “+” indicating that the crosslinked polyamine particles are held back by a sieve of the indicated mesh size and a “−” indicating that the crosslinked polyamine particles pass through a sieve of the indicated mesh size. Thus a crosslinked polyamine particle that passes through a No. 5 mesh sieve but is held back by a No. 20 mesh sieve is designated as being −5/+20. All references to mesh size described herein refer to mesh sizes that are U.S. Standard and in conformance with ASTM E-11. In some embodiments, from 75 wt. % to 100 wt. %, such as 80 wt. %, 85 wt. %, 90 wt. % or 95 wt. % of the crosslinked polyamine particles have a mesh size that is −5, −6, −7, −8, −10, 12, −14, −16, −18, −20, or −25. In some embodiments from 50 to 100 wt. % such as 55 wt. %, 60 wt. %, 65 wt. %, 70 wt. %, 75 wt. %, 80 wt. %, 85 wt. %, 90 wt. % or 95 wt. % of the crosslinked polyamine particles have a mesh size that is +60, +50, +45, +40, +35 or +30. In some embodiments, from 50 wt. % to 100 wt. % such as 55 wt. %, 60 wt. %, 65 wt. %, 70 wt. %, 75 wt. %, 80 wt. %, 85 wt. %, 90 wt. % or 95 wt. % of the crosslinked polyamine particles have a mesh size that is −5/+60, such as −6/+60, −7/+60, −8/+60, −10/+60, −12/+60, −14/+60, −16/+50, −18/+50, −20/+50, −25/+45, −25/+40, −25/+35 or −25/+30. In some embodiments, from 40 wt. % to 60 wt. % of the crosslinked polyamine particles have a mesh size that is −16/+40 mesh, such as −18/+35, −20/+35, −20/+30 or −20/+25.

In some embodiments of the invention, from 5 to 100 wt. % of the crosslinked polyamine particles, such as 10 to 90 wt. %, 20 to 80 wt. %, 30 to 70 wt. %, 40 to 60 wt. % or 50 wt. % of the crosslinked polyamine particles have a mesh size that +35 mesh, such as +30, +25, +20, +18, +16, or +14 mesh.

In some embodiments of the invention, greater than 10 wt. %, greater than 20 wt. %, greater than 30 wt. %, greater than 40 wt. %, greater than 50 wt. %, greater than 60 wt. %, greater than 70 wt. %, greater than 80 wt. %, greater than 90 wt. % or greater than 95 wt. % of the crosslinked polyamine particles have a mesh size that +35 mesh, such as +30, +25, +20, +18, +16, or +14 mesh.

In some embodiments of the invention, no more than 0 to 20 wt. %, such as no more than 5 to 15 wt. %, such as no more than 10 wt. % of the crosslinked polyamine particles have a mesh size that is −50. In some embodiments of the invention, no more than 0 to 25 wt. %, such as no more than 5 to 20 wt. %, such as no more than 10 wt. % or no more than 15 wt. % of the crosslinked polyamine particles have a mesh size that is −45. In some embodiments of the invention, no more than 0 to 35 wt. %, such as no more than 5 to 35 wt. %, such as no more than 10 wt. %, 15 wt. %, 20 wt. %, 25 wt. % or no more than 20 wt. % of the crosslinked polyamine particles have a mesh size that is −40. In some embodiments of the invention, no more than 0 to 45 wt. %, such as no more than 5 to 30 wt. %, such as no more than 10 wt. %, 15 wt. %, 20 wt. %, 25 wt. %, 30 wt. %, 35 wt. %, or no more than 40 wt. % have a mesh size that is −35.

In some embodiments, crosslinked polyamine particles of the invention may have any one or more of the particle size characteristics described herein prior to being formulated into a final dosage form, while in other embodiments, crosslinked polyamine particles of the invention may have any one or more of the particle size characteristics described herein when in a final dosage form. In some embodiments, any of the particle size characteristics described above may be determined prior to tableting. In other embodiments, any of the particle size characteristics described above may be determined after tableting has occurred.

Any suitable method of controlling or achieving the desired particle size may be used. For example, the particle size of the crosslinked polyamine particles may be controlled by controlling various polymerization process parameters such as temperature, monomer and crosslinker concentration, solvent, monomer to solvent ratio, pH, infusion rate, mixing rate, and by selecting the downstream process and processing parameters. For example, the particle size may be affected by the orifice size of a spray dryer nozzle and the height of a spray drying tower or the drying temperature. In addition, after polymerization, the crosslinked polyamine particles may be further processed to achieve the desired particle size such as ground using a grinder or a mill or selectively sieved. Any suitable method of controlling or achieving the desired particle size may be used. Specific suitable downstream processing methods include, but are not limited to grinding, wet or dry milling, spray drying, sieving, precipitation, and spray-freezing. In some embodiments, the down stream processing methods comprise wet milling.

In some embodiments, the crosslinked polyamine particles may have one or more of the following particle size characteristics, such as 1, 2, 3, 4, 5, 6, 7, 8, 9 or even all 10 of the following particle size characteristics:
a) a particle size distribution such that 75 vol. % or greater of the crosslinked polyamine particles have a size of between 250 μm and 4 mm;
b) a particle size distribution where from 5 vol. % to 100 vol. % of the crosslinked polyamine particles have a particle size of greater than 500 μm;
c) a particle size distribution such that no more than 20 vol. % of the crosslinked polyamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50; and/or
j) from 40 wt. % to 60 wt. % of the crosslinked polyamine particles have a mesh size that is −16/+40.

Thus, by way of example, in some embodiments, the crosslinked polyamines may have 3 of the above particle size characteristics such as a, e and h (or aeh) and would thus have a particle size distribution such that 75 vol. % or greater of the crosslinked polyamine particles have a size of between 250 μm and 4 mm, a particle size distribution such that the crosslinked polyamine particles have a d₉₀ value that is between 900 μm and 1600 μm and from 5 wt. % to 100 wt. % of the crosslinked polyamine particles have a mesh size that is +35. Accordingly it should be understood that the crosslinked polyamine particles may have any one or more of the above characteristics in any combination. Similarly, when any characteristics herein are provided in a list that includes “and/or” it should be understood that each and every possible permutation of combinations of those characteristics are specifically disclosed and included herein.

In addition, it should be understood that each of the characteristics identified herein by a letter such as “a)” may be any permutation of that same characteristic as discussed in the various detail paragraphs herein. For example, characteristic “a)” refers to a particle size distribution such that 75 vol. % or greater of the crosslinked polyamine particles have a size of between 250 μm and 4 mm. This reference however should be understood to encompass the detailed discussion of this characteristic above where it is shown that characteristic “a)” refers to particles having a particle size distribution such that 75 vol. % or greater, such as 80 vol. % or greater, 85 vol. % or greater, 90 vol. % or greater, 95 vol. % or greater, 99 vol. % or greater, or 100 vol. % of the crosslinked polyamine particles have a particle size between 250 μm and 4 mm, such as between 275 μm and 3.5 mm, between 300 μm and 3.0 mm, between 300 μm and 2.5 mm, between 300 μm and 2.0 mm, between 325 μm and 2.5 mm, between 350 μm and 2.0 mm, between 375 μm and 1.75 mm, between 400 μm and 1500 μm, between 425 μm and 1400 μm, between 450 μm and 1300 μm, between 475 μm and 1200 μm, between 500 μm and 1100 μm, or between 525 μm and 1075 μm. Each of the individual characteristics identified by letters in this application should be understood to refer to their detail paragraph or paragraphs discussed elsewhere in this application.

In some embodiments, crosslinked polyamine particles according to the invention exhibit special optical characteristics, such as optical density. In some embodiments, the crosslinked polyamine particles may have a mean gray value of greater than 180, such as a mean gray value of greater than 185, greater than 190, greater than 195, greater than 200, greater than 205, greater than 210, greater than 215 or greater than 220. In some embodiments, crosslinked polyamine particles according to the invention have a mean gray value that is between 180 and 230, such as between 185 and 225, between 190 and 215, between 190 and 210, between 195 and 205 or between 195 and 200. The mean gray value may be measured according to the techniques described in the Test Methods section below.

In some embodiments, the crosslinked polyamine particles described herein may comprise constituent particles or may comprise aggregates of constituent particles of the crosslinked polyamine polymers. In some embodiments, the constituent particles may have a particle size distribution such that greater than 70%, such as greater than 80 vol. %, such as greater than 85 vol. %, greater than 90 vol. %, greater than 95 vol. %, greater than 99 vol. % or 100 vol. % of the constituent particles have particle size between 10 μm and 850 μm, such as between 10 μm and 800 μm, between 10 μm and 750 μm, between 10 μm and 650 μm, between 10 μm and 550 μm, between 10 μm and 450 μm, between 10 μm and 400 μm, between 20 μm and 650 μm, between 30 μm and 550 μm, between 40 μm and 450 μm, between 50 μm and 400 μm, between 55 μm and 750 μm, between 55 μm and 650 μm, between 55 μm and 550 μm, between 55 μm and 500 μm, between 55 μm and 450 μm, between 55 μm and 400 μm, between 60 μm and 350 μm, between 65 μm and 300 μm, between 70 μm and 250 μm, between 75 μm and 200 μm, between 85 μm and 150 μm, between 90 μm and 125 μm or between 90 μm and 105 μm.

In some embodiments, the crosslinked polyamine particles described herein may comprise constituent particles or may comprise aggregates of constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm.

In some embodiments, the crosslinked polyamine particles described herein may comprise constituent particles or may comprise aggregates of constituent particles where the constituent particles have a particle size distribution such that the constituent particles have a d₁₀ value greater than 20 μm, greater than 25 μm, greater than 28 μm or greater than 30 μm.

In some embodiments, the crosslinked polyamine particles described herein may comprise constituent particles or may comprise aggregates of constituent particles where the constituent particles have a particle size distribution such that the constituent particles have a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise constituent particles or may comprise aggregates of constituent particles where the constituent particles have a particle size distribution such that the constituent particles have a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise constituent particles or may comprise aggregates of constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise constituent particles or may comprise aggregates of constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm and a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise constituent particles or may comprise aggregates of constituent particles where the constituent particles have a particle size distribution such that the constituent particles have a d₁₀ value greater than 20 μm, greater than 25 μm, greater than 28 μm or greater than 30 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise constituent particles or may comprise aggregates of constituent particles where the constituent particles have a particle size distribution such that the constituent particles have a d₁₀ value greater than 20 μm, greater than 25 μm, greater than 28 μm or greater than 30 μm and a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise constituent particles or may comprise aggregates of constituent particles where the constituent particles have a d₅₀ between 50 μm and 200 μm, such as between 50 μm and 175 μm, between 50 μm and 150 μm, between 50 μm and 120 μm, between 70 μm and 120 μm or between 70 μm and 100 μm.

In some embodiments, the crosslinked polyamine particles comprise 2 or more constituent particles, such as from 2 to 10,000 constituent particles, such as from 10 to 9000 constituent particles, from 100 to 8000 constituent particles, from 150 to 7000 constituent particles, from 200 to 6000 constituent particles, from 250 to 5000 constituent particles, from 275 to 4000 constituent particles, from 300 to 3500 constituent particles, from 350 to 3000 constituent particles, from 400 to 2500 constituent particles, from 450 to 2000 constituent particles, from 500 to 1500 constituent particles, from 600 to 1250 constituent particles, from 700 to 1000 constituent particles. In some embodiments, the crosslinked polyamine particles comprise from 500 to 1000 constituent particles.

In some embodiments, the crosslinked polyamine particles comprise aggregates of 2 or more constituent particles, such as from 2 to 10,000 constituent particles, such as from 10 to 9000 constituent particles, from 100 to 8000 constituent particles, from 150 to 7000 constituent particles, from 200 to 6000 constituent particles, from 250 to 5000 constituent particles, from 275 to 4000 constituent particles, from 300 to 3500 constituent particles, from 350 to 3000 constituent particles, from 400 to 2500 constituent particles, from 450 to 2000 constituent particles, from 500 to 1500 constituent particles, from 600 to 1250 constituent particles, from 700 to 1000 constituent particles. In some embodiments, the crosslinked polyamine particles comprise aggregates of from 500 to 1000 constituent particles.

In some embodiments, the crosslinked polyamine particles described herein may comprise particles which are formed by aggregating 2 or more constituent particles. In some embodiments, the constituent particles may have a particle size distribution such that greater than 70%, such as greater than 80 vol. %, such as greater than 85 vol. %, greater than 90 vol. %, greater than 95 vol. %, greater than 99 vol. % or 100 vol. % of the constituent particles have particle size between 10 μm and 850 μm, such as between 10 μm and 800 μm, between 10 μm and 750 μm, between 10 μm and 650 μm, between 10 μm and 550 μm, between 10 μm and 450 μm, between 10 μm and 400 μm, between 20 μm and 650 μm, between 30 μm and 550 μm, between 40 μm and 450 μm, between 50 μm and 400 μm, between μm 55 μm and 750 μm, between 55 μm and 650 μm, between 55 μm and 550 μm, between 55 μm and 500 μm, between 55 μm and 450 μm, between 55 μm and 400 μm, between 60 μm and 350 μm, between 65 μm and 300 μm, between 70 μm and 250 μm, between 75 μm and 200 μm, between 85 μm and 150 μm, between 90 μm and 125 μm or between 90 μm and 105 μm.

In some embodiments, the crosslinked polyamine particles described herein may comprise particles which are formed by aggregating 2 or more constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm.

In some embodiments, the crosslinked polyamine particles described herein may comprise particles which are formed by aggregating 2 or more constituent particles where the constituent particles have a particle size distribution such that the constituent particles have a d₁₀ value greater than 20 μm, greater than 25 μm, greater than 28 μm or greater than 30 μm.

In some embodiments, the crosslinked polyamine particles described herein may comprise particles which are formed by aggregating 2 or more constituent particles where the constituent particles have a particle size distribution such that the constituent particles have a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise particles which are formed by aggregating 2 or more constituent particles where the constituent particles have a particle size distribution such that the constituent particles have a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise particles which are formed by aggregating 2 or more constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise particles which are formed by aggregating 2 or more constituent particles where the constituent particles have a particle size distribution such that the constituent particles have a d₁₀ value between 20 μm and 100 μm, such as between 20 μm and 70 μm, between 25 μm and 60 μm, between 28 μm and 53 μm, or between 30 μm and 50 μm and a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise particles which are formed by aggregating 2 or more constituent particles where the constituent particles have a particle size distribution such that the constituent particles have a d₁₀ value greater than 20 μm, greater than 25 μm, greater than 28 μm or greater than 30 μm and a d₉₀ value that is between 120 μm and 450 μm, such as between 150 μm and 400 μm, between 175 μm and 350 μm, between 175 μm and 300 μm, between 175 μm and 275 μm or between 175 μm and 250 μm.

In some embodiments, the crosslinked polyamine particles described herein may comprise particles which are formed by aggregating 2 or more constituent particles where the constituent particles have a particle size distribution such that the constituent particles have a d₁₀ value greater than 20 μm, greater than 25 μm, greater than 28 μm or greater than 30 μm and a d₉₀ value that is less than 450 μm, such as less than 425 μm, less than 400 μm, less than 375 μm, less than 350 μm, less than 325 μm, less than 300 μm, less than 275 μm or less than 250 μm.

In some embodiments, the crosslinked polyamine particles described herein may comprise particles which are formed by aggregating 2 or more constituent particles where the constituent particles have a d₅₀ between 50 μm and 200 μm, such as between 50 μm and 175 μm, between 50 μm and 150 μm, between 50 μm and 120 μm, between 70 μm and 120 μm or between 70 μm and 100 μm.

In some embodiments, may comprise particles which are formed by aggregating 2 or more constituent particles, such as from 2 to 10,000 constituent particles, such as from 10 to 9000 constituent particles, from 100 to 8000 constituent particles, from 150 to 7000 constituent particles, from 200 to 6000 constituent particles, from 250 to 5000 constituent particles, from 275 to 4000 constituent particles, from 300 to 3500 constituent particles, from 350 to 3000 constituent particles, from 400 to 2500 constituent particles, from 450 to 2000 constituent particles, from 500 to 1500 constituent particles, from 600 to 1250 constituent particles, from 700 to 1000 constituent particles. In some embodiments, the crosslinked polyamine particles comprise from 500 to 1000 constituent particles.

In some embodiments, aggregating 2 or more constituent particles includes hydrating constituent particles, such as suspending, forming a suspension of or forming a re-suspension of constituent particles in water. In some embodiments, forming a suspension of or forming a re-suspension of constituent particles includes protonating, such as carbonating, at least a portion of the crosslinked polyamine particles. In some embodiments, forming includes making a gel from constituent particles. In some embodiments, the gel may be dried and/or the gel may be ground, milled or wet milled.

In some embodiments, the crosslinked polyamine particles according to the invention may have an in vitro competitive phosphate binging capacity at 60 minutes that is greater than 1.2 mmol phosphate/g of polymer, such as greater than 1.25 mmol/g, greater than 1.30 mmol/g, greater than 1.35 mmol/g, greater than 1.4 mmol/g, greater than 1.5 mmol/g, greater than 1.6 mmol/g, greater than 1.7 mmol/g, greater than 1.8 mmol/g, greater than 1.9 mmol/g or greater than 2.0 mmol/g. In some embodiments, the crosslinked polyamine particles according to the invention may have an in vitro competitive phosphate binging capacity at 60 minutes that is between 1.2 mmol/g and 10 mmol/g, such as between 1.2 mmol/g and 7.5 mmol/g, between 1.2 mmol/g and 5.0 mmol/g, between 1.2 mmol/g and 4.0 mmol/g, between 1.25 mmol/g and 4.0 mmol/g, between 1.3 mmol/g and 4.0 mmol/g, between 1.35 mmol/g and 4.0 mmol/g, between 1.4 mmol/g and 4.0 mmol/g, between 1.5 mmol/g and 4.0 mmol/g, between 1.6 mmol/g and 4.0 mmol/g, between 1.7 mmol/g and 4.0 mmol/g, or between 1.8 mmol/g and 4.0 mmol/g.

In some embodiments, the invention is, consists essentially of, or comprises crosslinked polyamine particles, a pharmaceutical composition comprising crosslinked polyamine particles or a method for removing a compound or ion, such as a phosphorous-containing compound or a phosphorous-containing ion (e.g. phosphate), from the gastrointestinal tract of an animal by administering an effective amount of crosslinked polyamine particles or a pharmaceutical composition comprising crosslinked polyamine particles, where the crosslinked polyamine particles have one or more of the following characteristics:
a) a particle size distribution such that 75 vol. % or greater of the crosslinked polyamine particles have a size of between 250 μm and 4 mm;
b) a particle size distribution where from 5 vol. % to 100 vol. % of the crosslinked polyamine particles have a particle size of greater than 500 μm;
c) a particle size distribution such that no more than 20 vol. % of the crosslinked polyamine particles have a particle size less than 300 μm;
d) a particle size distribution such that the crosslinked polyamine particles have a d₁₀ value that is between 250 μm and 750 μm
e) a particle size distribution such that the crosslinked polyamine particles have a d₉₀ value that is between 900 μm and 1600 μm;
f) a particle size distribution such that the crosslinked polyamine particles have a d₅₀ between 450 μm and 1100 μm;
g) from 75 wt. % to 100 wt. % of the crosslinked polyamine particles have a mesh size that is −5/+60;
h) from 5 wt. % to 100 wt. % of the crosslinked polyamine particles have a mesh size that is +35;
i) no more than 20 wt. % of the crosslinked polyamine particles have a mesh size that is −50;
j) from 40 wt. % to 60 wt. % of the crosslinked polyamine particles have a mesh size that is −16/+40;
k) a mean gray value greater than 180; 1) comprises 2 or more constituent particles; and/or
m) a competitive phosphate binding capacity at 60 minutes of greater than 1.2.

In general, the invention provides for crosslinked polyamine particles, compositions comprising crosslinked polyamine particles and methods of making and using crosslinked polyamine particles where the crosslinked polyamine particles may comprise any suitable crosslinked polyamine particles. In general, suitable crosslinked polyamines include compounds or polymers having multiple amine groups, where the compounds or polymers have been crosslinked and where the crosslinked compounds and polymers are suitable as pharmaceuticals. Examples of some crosslinked polyamines that may be used with the invention may be found, for instance, in U.S. Application Ser. Nos. 60/847,905; 60/841,566; 60/849,434; 60/853,440; 60/874,715; 60/902,848; 60/905,595 and 60/924,043, the entire contents of each of which is hereby incorporated by reference in their entirety.

In some embodiments, the crosslinked polyamine particles may comprise crosslinked polyamine dendrimers. In some embodiments, the crosslinked polyamine particles may comprise crosslinked hyperbranched polyamine particles. Non-limiting examples of some suitable crosslinked polyamines are described below.

In some embodiments, the invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound comprises or is derived from one or more substituted or unsubstituted polyhydroxy compounds and one or more substituted or un-substituted, α, β unsaturated nitriles.

In some embodiments, the invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound comprises an amine dendrimer or residue thereof, the dendrimer having a core that is a residue of one or more substituted polyhydroxy compounds and a residue of one or more substituted or un-substituted, α, β unsaturated nitriles.

In some embodiments, the invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound is represented by Formula I, as follows:

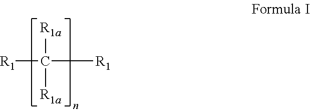

wherein n independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₁ independently represents a hydrogen radical, a hydroxyl radical or —OR₃; R₁ₐ independently represents R₁, —R₂OH or —R₂OR₃; with the proviso that the amine compound includes at least one moiety represented by R₃; R₂ independently represents a substituted or un-substituted, branched or unbranched alkyl radical, for example a C₁ to C₂₀ alkyl radical, such as a C₁, C₂, C₃, C₄, C₅ or C₆ radical; and R₃ independently represents a group represented by the following Formula II:

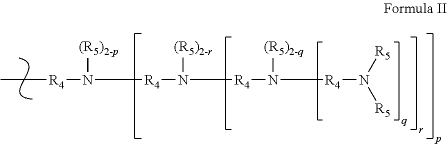

wherein p, q and r independently represent an integer from 0-2, for example, 0, 1 or 2; R₄ independently represents

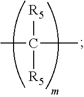

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In some embodiments, the present invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound is represented by Formula I and where R₃ independently represents a group represented by the following Formula III or Formula IV:

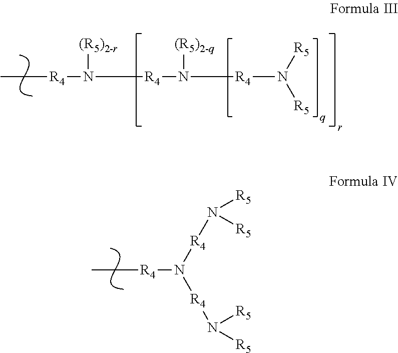

where q, r, R₄ and R₅ are as defined above.

In some embodiments, the present invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound is represented by Formula V, as follows:

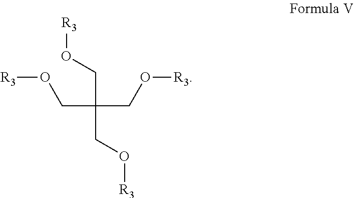

where R₃ independently represents a group represented by Formula II, Formula III, or Formula IV as defined above.

In some embodiments, the present invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound represented by Formula VI, as follows:

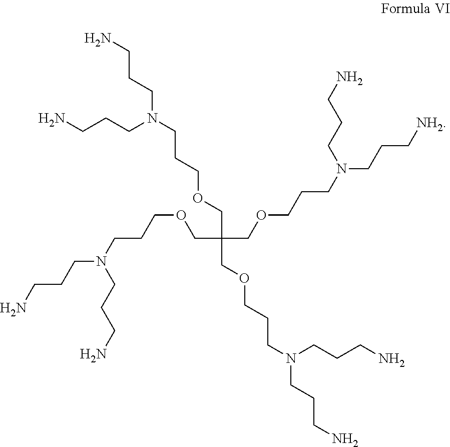

In some embodiments, the present invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound is represented by Formula VII, as follows:

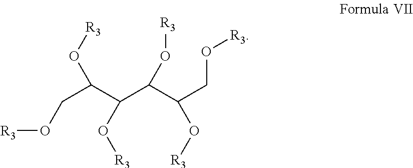

wherein R₃ independently represents a group represented by Formula II, Formula III, or Formula IV as defined above.

In some embodiments, the present invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound is represented by Formula VIII, as follows:

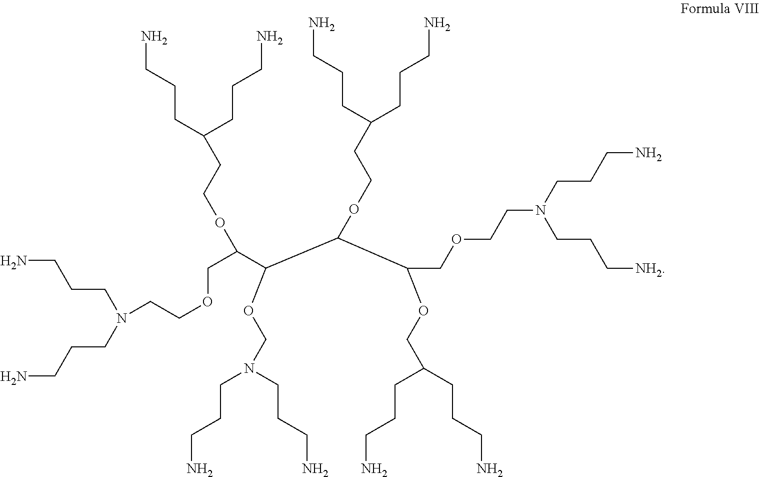

In some embodiments, the present invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound comprises one or more sugar alcohols substituted with an amine group represented by the following Formula IX:

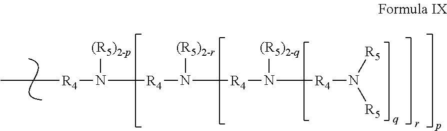

wherein p, q and r independently represent an integer from 0-2, for example, 0, 1 or 2; R₄ independently represents

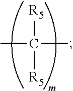

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In some embodiments, the present invention comprises crosslinked polyamine particles comprising or derived from an amine compound or a residue thereof, a pharmaceutical composition comprising crosslinked polyamine particles comprising or derived from said amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amine compound comprises an amine dendrimer or residue thereof, the dendrimer having a core that is a residue of one or more sugars or sugar alcohols and a residue of one or more substituted or un-substituted α, β unsaturated nitriles.

Polyhydroxy compounds that may be used as cores for, or in the preparation of crosslinked polyamines and compositions according to some embodiments of the invention include straight chain, branched, cyclic, alicyclic, aromatic, and heterocyclic polyhydric alcohols, such as 1,4-butanediol, 1,5-pentanediol, 1,6-hexanediol, 1,6-cyclohexanedimethanol, 2-methyl-1,3-propanediol, 2-methyl-2-ethyl-1,3-propanediol, 2-ethyl-2-butyl-1,3-propanediol, neopentyl glycol, dimethylolpropane, 1,1-dimethylolcyclohexane, glycerol, trimethylolethane, trimethylolpropane, diglycerol, ditrimethylolethane, ditrimethylolpropane, pentaerythritol, dipentaerythritol, inositol, sugars and sugar alcohols.

Examples of some aromatic, alicyclic and heterocyclic groups that may be substituted with at least 2 hydroxyl groups to form suitable polyhydroxy compounds include cyclopropyl, cyclobutyl, cyclopentyl, cyclohexyl, piperidinyl, piperizinyl, thiazolidinyl, imidazolidinyl, pyranyl, tetrahydrofuranyl, oxanyl, benzyl, pyridinyl, imidazolyl, pyrazolyl, thiazolyl, oxazolyl, pyrimidinyl, dioxanyl, quinizolinyl, indolinyl, benzothiazolyl, benzooxazolyl, pyrazinyl, furanyl, thenyl, naphthalenyl and the like.

Non-limiting examples of some suitable cyclic polyhydroxy compounds include: cyclohexane-1,2-diol, cyclohexane-1,3-diol, cyclohexane-1,4-diol, cyclohexane-1,2,3-triol, cyclohexane-1,2,4-triol, cyclohexane-1,3,4-triol, cyclohexane-1,3,5-triol, cyclohexane-1,2,3,4-tetraol, cyclohexane-1,3,4,5-tetraol, cyclohexane-1,2,3,4,5-pentaol, cyclohexane-1,2,3,4,5,6-hexaol, cyclopentane-1,2-diol, cyclopentane-1,3-diol, cyclopentane-1,2-diol, cyclopentane-1,2,3-triol, cyclopentane-1,2,4-triol, cyclopentane-1,2,3,4-tetraol, cyclopentane-1,2,3,4,5-pentaol, benzene-1,2-diol, benzene-1,3-diol, benzene-1,4-diol, benzene-1,2,3-triol, benzene-1,2,4-triol, benzene-1,3,4-triol, benzene-1,3,5-triol, benzene-1,2,3,4-tetraol, benzene-1,3,5-triol, benzene-1,2,3,4-tetraol, benzene-1,2,3,5-tetraol, benzene-1,2,4,5-tetraol, benzene-1,2,3,4,5-pentaol, and benzene-1,2,3,4,5,6-hexaol.

Sugars and sugar alcohols that are suitable for use alone or in combination in some embodiments of the crosslinked polyamine polymers or compositions of the present invention include monosaccharides and sugar alcohols derived from monosaccharides. Examples of such compounds include sugars or sugar alcohols comprising or derived from aldoses and ketoses including those comprising or derived from monoses, dioses, trioses, tetroses, pentoses, hexoses, heptoses, octoses and nonoses. The aldoses and ketoses may be fully or partially hydrogenated, and may be substituted, including replacement of one or more hydroxyl groups on the aldose or ketose with one or more hydrogen groups to form the corresponding deoxyaldose or deoxyketose, provided that at least one alcohol group remains and substitution of one or more hydroxyl groups with one or more amine groups to form the corresponding amino sugar. Specific non-limiting examples of some suitable substituted or unsubstituted aldoses and ketoses include: erythrose, threose, ribose, deoxyribose, arabinose, xylose, lyxose, allose, altrose, glucose, mannose, gulose, idose, galactose, talose, ribulose, rhamnose, fucose, ribodesose, xylulose, fructose, psicose, tagatose, mannoheptulose, sedoheptulose, sorbose, pentaerythrose, octolose, sialose, glucosamine, glucosylamine, mannosamine, galactosamine, allosamine, altrosamine, ribosamine, arabinosamine, gulosamine, idosamine, talosamine, xylosamine, lyxosamine, sorbosamine, tagatosamine, psicosamine, fructosamine, and sialic acids, including both the D and L forms of each, α and β forms of each, partially or fully hydrogenated derivatives thereof, or combinations thereof. Non-limiting examples of some suitable sugar alcohols include sorbitol, mannitol, maltitol, xylitol, erythritol, lactitol, galactitol, dulcitol, arabitol, threitol, arabinitol, ribitol, and rhamnitol.

In some embodiments, suitable polyhydroxy compounds include one or more substituted or unsubstituted cyclic sugars or cyclic sugar alcohols such as cyclic forms of aldoses and ketoses, including cyclic forms of the aldoses and ketoses described above. Other suitable polyols that may be used alone or in combination include substituted or unsubstituted polysaccharides, including disaccharides and oligosaccharides, including hetero and homopolysaccharides derived from cyclic forms of the aldoses and ketoses described herein. Such polysaccharides may be unbranched or branched and may include α and/or β glycosidic bonds such as, for example, α(1→4), α(1→1), α(1→6), α(1→3), β(1→3) and/or β(1→4) glycosidic bonds. In unsubstituted form, polysaccharides may have the general formula Cₙ(H₂O)ₙ₋₁, where n is from 6-3000. Non-limiting examples of some substituted or unsubstituted polysaccharides include: sucrose, maltose, chitobiose, laminarbiose, kojibiose, xylobiose, trehalose, saccharose, cellobiose, gentiobiose, lactose, melibiose, raffinose, gentianose, melizitose, stachyose, inulin, methyl-α-glucopyranoside, amylosamine, maltosamine, agarosamine, cellulosamine, saccharosamine, starches, amylose, amylopectin, pectins/pectic polysaccharides, arabingalactans, mannans, mucopolysaccharides, hyaluronic acid, heparin, glucomannans, celluloses, chitins, glycogen, callose, laminarin, xylan and galactomannan.

Examples of some suitable polyhydroxy compounds include the following compounds:

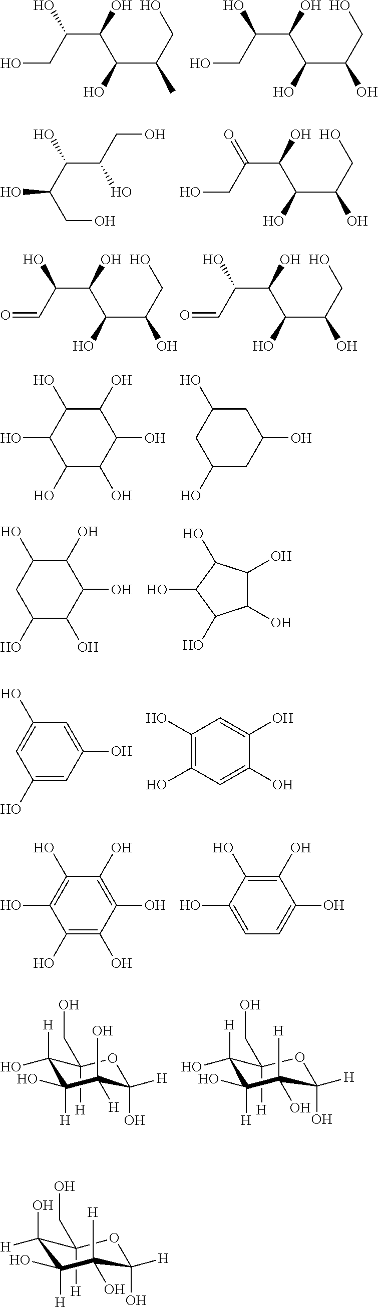

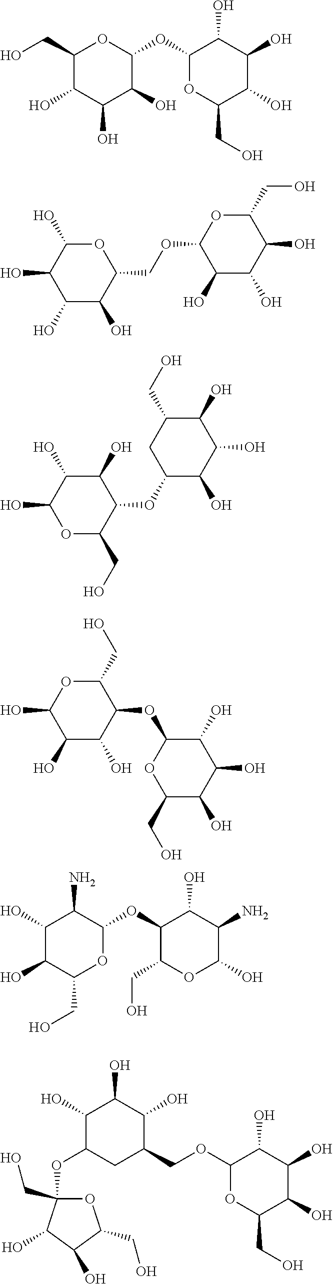

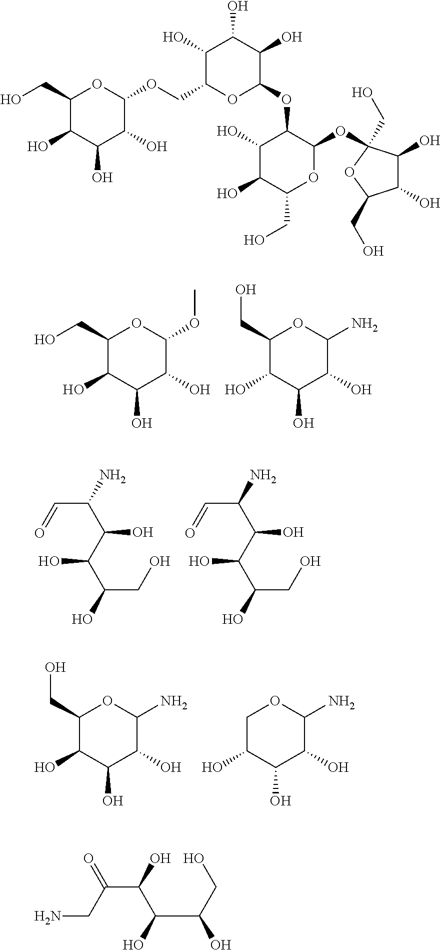

In some embodiments, the invention comprises crosslinked polyamine particles comprising or derived from at least one amido-amine compound or a residue thereof, a pharmaceutical composition comprising crosslinked polyamine particles comprising or derived from said at least one amido-amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the at least one amido-amine compound or residue thereof may be derived from a multi-amine and a multifunctional compound, where the multifunctional compound comprises two or more amine-reactive groups. In some embodiments, the amine reactive groups are independently selected from the group consisting of vinyl groups, carboxylic acid groups and ester groups and combinations thereof.

In some embodiments, the multifunctional compound comprising two or more amine-reactive groups is selected from the group consisting of

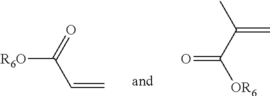

where R₆ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical, such as, for example,

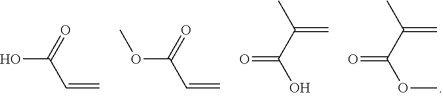

In some embodiments, the multi-amine is selected from the group consisting of:

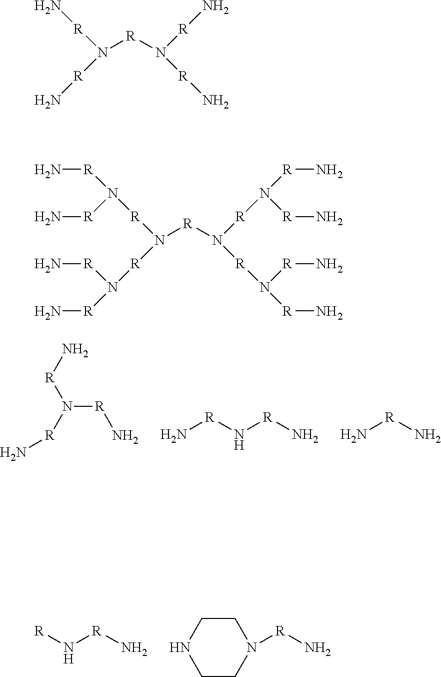

and combinations thereof, wherein R independently represents a branched or unbranched, substituted or un-substituted alkyl radical, for example a C₁ to C₂₀ alkyl radical, such as a C₁, C₂, C₃, C₄, C₅ or C₆ radical, such as, for example,

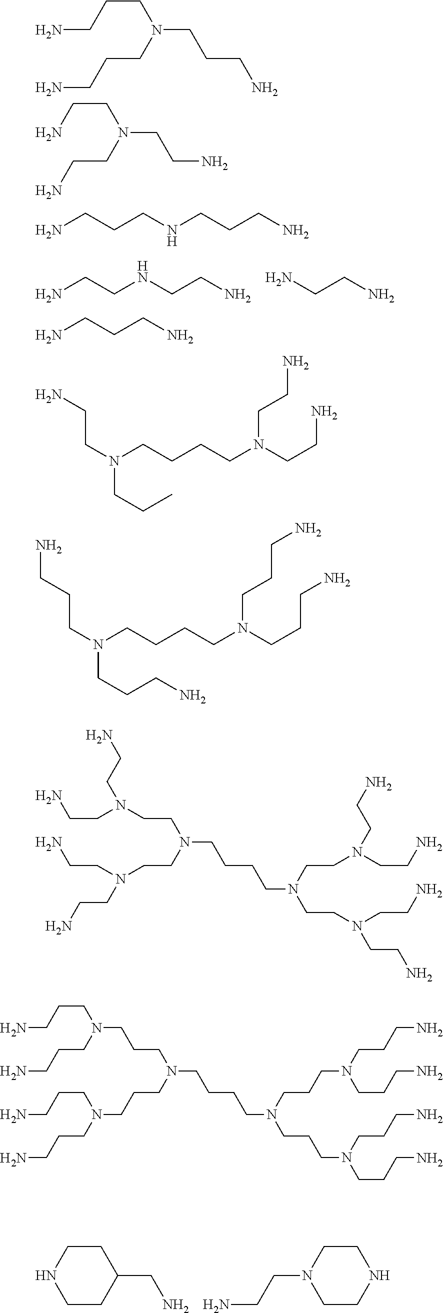

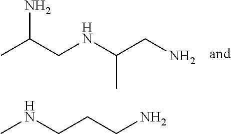

and combinations thereof.

In some embodiments, the invention comprises crosslinked polyamine particles comprising or derived from at least one amido-amine compound or a residue thereof, a pharmaceutical composition comprising crosslinked polyamine particles comprising or derived from said at least one amido-amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the at least one amido-amine compound or residue thereof is derived from compounds according to the following Formulas X and XI:

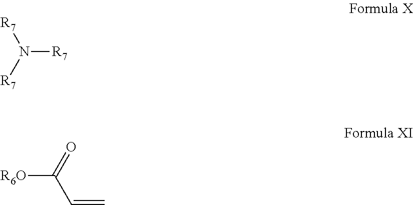

wherein R₇ independently represents a hydrogen radical, —RNH₂, —R—N—(R—NH₂)₂ or —R—N—(R—N—(R—NH₂)₂)₂, wherein R independently represents a branched or unbranched, substituted or un-substituted alkyl radical for example a C₁ to C₂₀ alkyl radical, such as a C₁, C₂, C₃, C₄, C₅ or C₆ radical, with the proviso that at least one R₇ is not a hydrogen radical and R₆ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical.

In some embodiments the compound according to Formula X is selected from the multi-amines described above.

In some embodiments, the invention comprises crosslinked polyamine particles comprising or derived from at least one amido-amine compound or a residue thereof, a pharmaceutical composition comprising crosslinked polyamine particles comprising or derived from said at least one amido-amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the at least one amido-amine compound or residue thereof is represented by the following Formula XII:

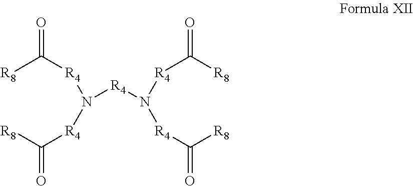

where R₈ independently represents a group represented by the following Formula XIII:

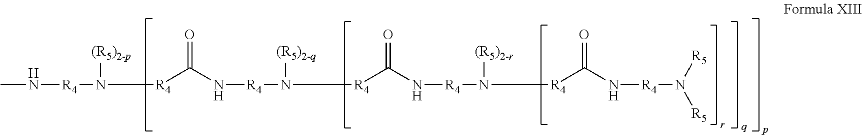

where p, q and r independently represent an integer from 0-2, for example 0, 1 or 2; R₄ independently represents

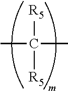

where m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof. Examples of such compounds include compounds according to Formulas XIV, XV or XVI:

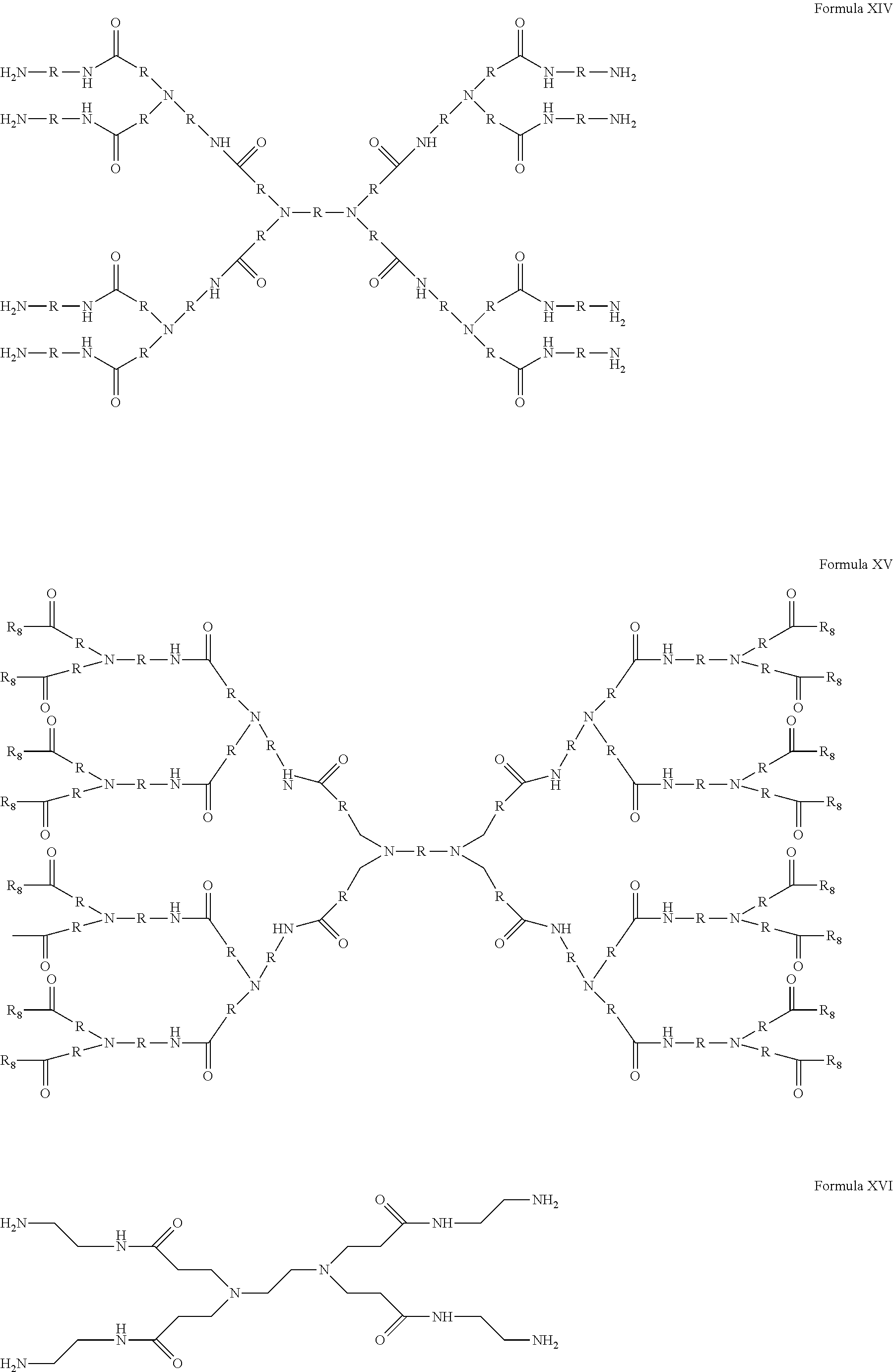

where R independently represents a branched or unbranched, substituted or un-substituted alkyl radical, for example a C₁ to C₂₀ alkyl radical, such as a C₁, C₂, C₃, C₄, C₅ or C₆ radical.

In some embodiments, the invention comprises crosslinked polyamine particles comprising or derived from at least one amido-amine compound or a residue thereof, a pharmaceutical composition comprising crosslinked polyamine particles comprising or derived from said at least one amido-amine compound or a residue thereof or a method of using the same in a therapeutically effective amount to remove a compound or ion, such as a phosphorous-containing compound or a phosphorous-containing ion (e.g. phosphate), from the gastrointestinal tract of an animal where the at least one amido-amine compound or residue thereof is represented by the following Formula XVII:

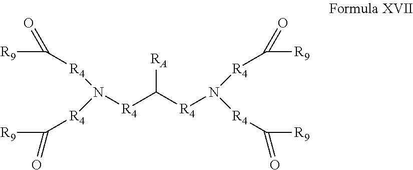

where R₉ independently represents a group represented by the following Formula XVIII:

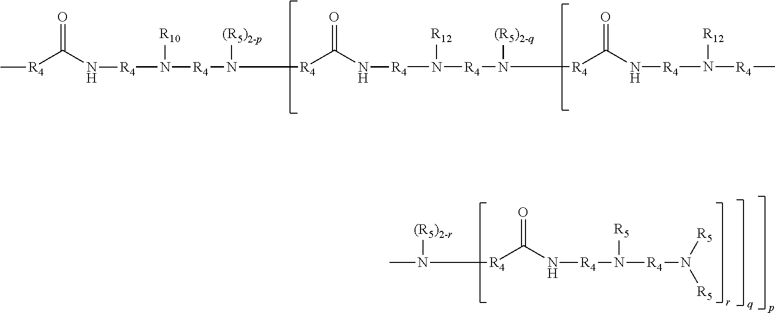

where p, q and r independently represent an integer from 0-2; R₄ independently represents:

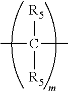

where m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof; R_{A} independently represents an R₅ group or a —R₄—CO—R₉ group; R₁₀ independently represents an R₅ group or independently represents a group according to the following Formula XIX:

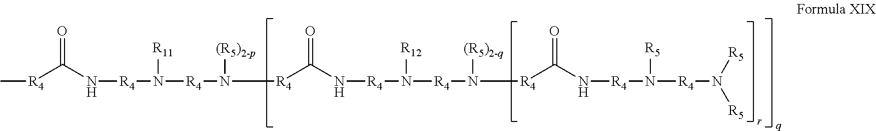

where R₁₁ independently represents an R₅ group or independently represents a group according to the following Formula XX:

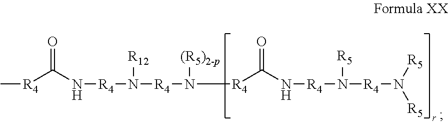

where R₁₂ independently represents an R₅ group or independently represents a group according to the following Formula XXI:

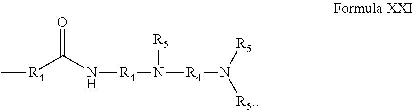

Examples of such compounds include, for example, compounds represented by the following Formula XXII:

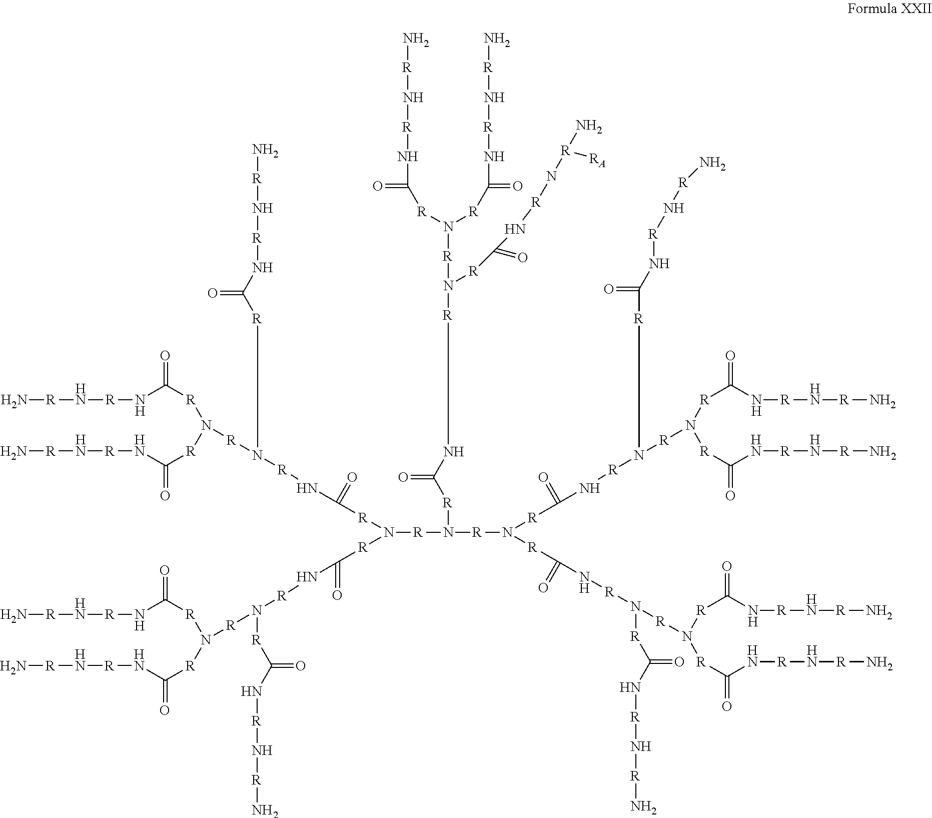

where R independently represents a branched or unbranched, substituted or un-substituted alkyl radical, for example a C₁ to C₂₀ alkyl radical, such as a C₁, C₂, C₃, C₄, C₅ or C₆ radical.

In some embodiments, the invention comprises crosslinked polyamine particles comprising or derived from at least one amido-amine compound or a residue thereof, a pharmaceutical composition comprising crosslinked polyamine particles comprising or derived from said at least one amido-amine compound or a residue thereof or a method of using the same in a therapeutically effective amount to remove a compound or ion, such as a phosphorous-containing compound or a phosphorous-containing ion (e.g. phosphate), from the gastrointestinal tract of an animal, where the amido-amine compound is derived from compounds according to the following Formulas XI and XXIII:

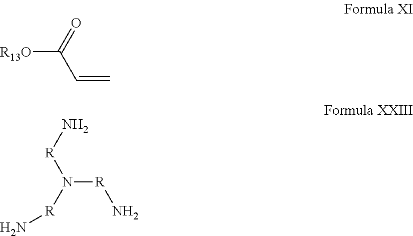

wherein R independently represents a branched or unbranched, substituted or un-substituted alkyl radical, for example a C₁ to C₂₀ alkyl radical, such as a C₁, C₂, C₃, C₄, C₅ or C₆ radical; R₁₃ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical.

In some embodiments, the compound according to Formula XXIII comprises:

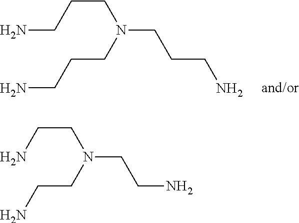

In some embodiments, the invention comprises crosslinked polyamine particles comprising or derived from at least one amido-amine compound or a residue thereof, a pharmaceutical composition comprising crosslinked polyamine particles comprising or derived from said at least one amido-amine compound or a residue thereof or a method of using the same in a therapeutically effective amount to remove a compound or ion, such as a phosphorous-containing compound or a phosphorous-containing ion (e.g. phosphate), from the gastrointestinal tract of an animal, where the amido-amine compound is derived from compounds according to the following Formulas XI and XXIV:

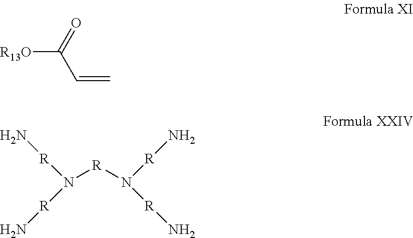

wherein R independently represents a branched or unbranched, substituted or un-substituted alkyl radical, for example a C₁ to C₂₀ alkyl radical, such as a C₁, C₂, C₃, C₄, C₅ or C₆ radical; R₁₃ independently represents a hydrogen radical or a branched or unbranched, substituted or un-substituted alkyl radical, for example a C₁ to C₂₀ alkyl radical, such as a C₁, C₂, C₃, C₄, C₅ or C₆ radical.

In some embodiments, the compound according to Formula XXIV comprises:

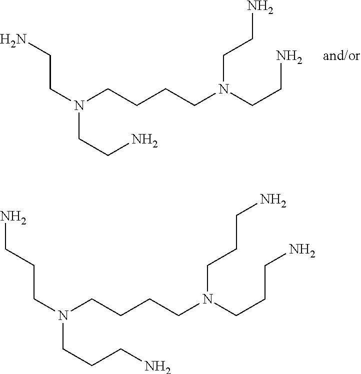

In some embodiments, the invention comprises crosslinked polyamine particles comprising or derived from at least one amido-amine compound or a residue thereof, a pharmaceutical composition comprising crosslinked polyamine particles comprising or derived from said at least one amido-amine compound or a residue thereof or a method of using the same in a therapeutically effective amount to remove a compound or ion, such as a phosphorous-containing compound or a phosphorous-containing ion (e.g. phosphate), from the gastrointestinal tract of an animal, where the amido-amine compound comprises an amido-amine dendrimer or residue thereof, the dendrimer having a core that is a residue of one or more multi-amine compounds and a residue of one or more substituted or un-substituted α, β unsaturated carboxylic acids or esters.

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide compound is represented by Formula XXV, as follows:

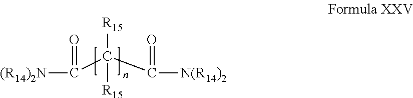

wherein n independently represents an integer from 0-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₁₅ independently represents a hydrogen radical, a hydroxyl radical, —OR₁₆, —R₁₇OH, —R₁₇OR₁₆, or C(O)N(R₁₄)₂; R₁₄ independently represents a hydrogen radical, a hydroxyl radical, —OR₁₆, or a branched or unbranched substituted C₁-C₁₀, such as a C₁, C₂, C₃, C₄, C₅, C₆, C₇, C₈, C₉, C₁₀, alkyl radical, wherein one or more carbon atoms of the alkyl radical may be partially or fully substituted with —OH and/or —OR₁₆ groups, for example a C₃-C₈ branched alkyl radical having more than one substitution, such as C₄-C₇ branched alkyl substituted with 2 or more —OH and/or —OR₁₆ groups, or a C₃ branched alkyl substituted with 3 or more —OH and/or —OR₁₆ groups; R₁₇ independently represents a substituted or unsubstituted, branched or unbranched alkyl radical; and R₁₆ is independently represented by the following Formula XXVI:

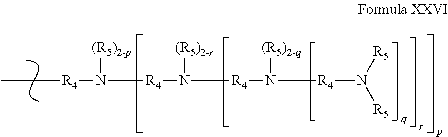

wherein p, q and r independently represent an integer from 0-2, such as 0, 1 or 2; R₄ independently represents

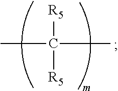

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide compound is represented by Formula XXV, wherein R₁₄ independently represents a branched or unbranched substituted C₁-C₁₀ alkyl radical that is partially or fully substituted with 1-20, for example 2-10, 2-6, 2-4, such as 1, 2, 3, 4, 5, 6, 7, 8, 9, 10, 11, 12, 13, 14, 15, 16, 17, 18, 19 or 20, —OH and/or OR₁₆ groups.

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide compound is represented by Formula XXV, and where R₁₆ independently represents a group represented by the following Formula XXVII:

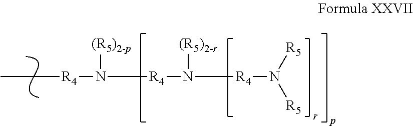

where p, r, R₄ and R₅ are as defined above.

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide compound is represented by Formula XXV, where R₁₆ independently represents a group represented by the following Formula XXVIII:

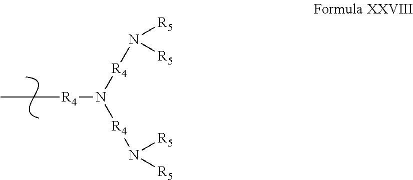

where R₄ and R₅ are as defined above.

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide compound is represented by Formula XXIX, as follows:

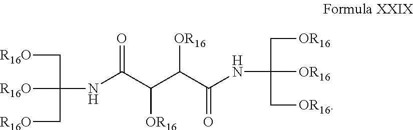

where R₁₆ independently represents a group represented by Formula XXVI, Formula XXVII, or Formula XXVIII as defined above.

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide compound is represented by Formula XXX, as follows:

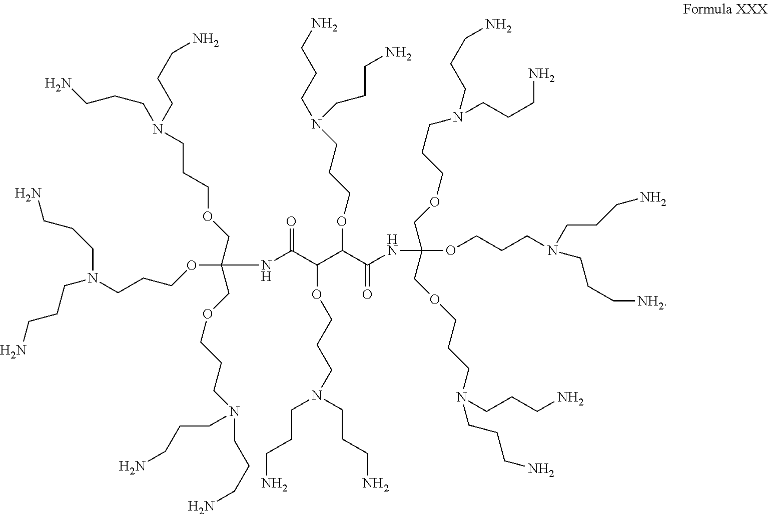

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide compound comprises a substituted amide polyol or residue thereof. The amide polyol may comprise a residue of a substituted or unsubstituted organic polyacid or ester thereof and a residue of a substituted or unsubstituted amine polyol. The amide polyol may be substituted with one or more groups represented by Formula XXVI, Formula XXVII, or Formula XXVIII as defined above.

In some embodiments, the present invention comprises crosslinked polyamine particles comprising or derived from an amide compound or a residue thereof, a pharmaceutical composition comprising crosslinked polyamine particles comprising or derived from said amide compound or a residue thereof or a method of using the same in a therapeutically effective amount to remove a compound or ion, such as a phosphorous-containing compound or a phosphorous-containing ion (e.g. phosphate), from the gastrointestinal tract of an animal where the amide polymer comprises at least one amide compound or residue thereof, where the amide compound comprises an amide dendrimer or residue thereof, the amide dendrimer comprising a substituted amide polyol or residue thereof and a residue or one or more substituted or unsubstituted α, β unsaturated nitriles or residues thereof. In some embodiments, the amide polyol may comprise a residue of a substituted or unsubstituted organic polyacid or ester thereof and a residue of a substituted or unsubstituted amine polyol.

In some embodiments, examples of some suitable amine polyols include amine-substituted straight chain, branched, cyclic, alicyclic, aromatic, and heterocyclic polyhydric alcohols, including the polyhydric alcohols described above.

In some embodiments, organic polyacids and/or esters thereof may be used to form the cores for, or in the preparation of, the crosslinked polyamines and compositions according to some embodiments of the invention. Esters of all of the organic polyacids may be used instead of, or in conjunction with, the organic polyacids, including polyacids that are partially and fully esterified. Examples of the polyacids include any organic polyacids, including diacids, triacids, tetracids, pentacids and hexacids. Examples of some polyacids that may be used include substituted or un-substituted methanetetracarboxylic acid, ethane-1,1,2,2-tetracarboxylic acid, oxalic acid, malonic acid, succinic acid, fumaric acid, maleic acid, glutaric acid, adipic acid, pimelic acid, suberic acid, azelaic acid, sebacic acid, tartaric acid, tartronic, 3-hydroxypentanedioc acid, 3,4-hydroxyhexanedioc acid, glucaric acid, mucic acid, galactaric acid, xylaric acid, aspartic acid, 2-amino malonic acid, citric acid, ethylenediaminetetraacetic acid. In some embodiments, the organic polyacids include one or more substitutions, where the substitutions comprise hydroxyl and/or amine groups.

In some embodiments, suitable organic polyacids that may be used to form the cores for, or in the preparation of, amide compounds, amide polymers, polymer networks and compositions according to some embodiments of the invention include aldaric acids having the following general formula:

HOOC—(CHOH)_{w}—COOH

wherein w represents an integer from 1 to 20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20. Additional examples of suitable aldaric acids include diacids formed from any of the sugar alcohols as mentioned above. In some embodiments, one or more of the non-acid hydroxyl groups of the aldaric acids may be replaced with an amine group.

In some embodiments, suitable organic polyacids may be cyclic polyacids such as aromatic, alicyclic or heterocyclic polyacids having a 3, 4, 5 or 6 membered ring or rings that are partially or fully substituted with carboxylic acid groups. For example, a 3-membered polyacid ring may have 2 or 3 carboxylic acid groups, a 6-membered polyacid ring may have 2, 3, 4, 5, or 6 carboxylic acid groups and a naphthalene group may have 2, 3, 4, 5, 6, 7 or 8 carboxylic acid groups. The heterocyclic organic polyacids may be aromatic or non-aromatic and may have up to four heteroatoms selected from N, O and S and combinations thereof. The cyclic polyacids may additionally have non-acid substitutions on the rings including, for example, —OH groups.

Examples of some aromatic, alicyclic and heterocyclic groups that may be substituted with at least 2 carboxylic acids to form suitable organic polyacids include cyclopropyl, cyclobutyl, cyclopentyl, cyclohexyl, piperidinyl, piperizinyl, thiazolidinyl, imidazolidinyl, pyranyl, tetrahydrofuranyl, oxanyl, benzyl, pyridinyl, imidazolyl, pyrazolyl, thiazolyl, oxazolyl, pyrimidinyl, dioxanyl, quinizolinyl, indolinyl, benzothiazolyl, benzooxazolyl, pyrazinyl, furanyl, thenyl, naphthalenyl and the like.

Non-limiting examples of some suitable cyclic polyacids include: cyclohexane-1,2-dicarboxylic acid, cyclohexane-1,3-dicarboxylic acid, cyclohexane-1,4-dicarboxylic acid, cyclohexane-1,2,3-tricarboxylic acid, cyclohexane-1,2,4-tricarboxylic acid, cyclohexane-1,3,4-tricarboxylic acid, cyclohexane-1,3,5-tricarboxylic acid, cyclohexane-1,2,3,4-tetracarboxylic acid, cyclohexane-1,3,4,5-tetracarboxylic acid, cyclohexane-1,2,3,4,5-pentacarboxylic acid, cyclohexane-1,2,3,4,5,6-hexacarboxylic acid, cyclopentane-1,2-dicarboxylic acid, cyclopentane-1,3-dicarboxylic acid, cyclopentane-1,2-dicarboxylic acid, cyclopentane-1,2,3-tricarboxylic acid, cyclopentane-1,2,4-tricarboxylic acid, cyclopentane-1,2,3,4-tetracarboxylic acid, cyclopentane-1,2,3,4,5-pentacarboxylic acid, phthahlic acid, isophthalic acid, terephthalic acid, hemimellitic acid, trimellitic acid, trimesic acid, benzene-1,2,3,4-tetracarboxylic acid, benzene-1,2,3,5-tetracarboxylic acid, pyromellitic acid, benzene-1,2,3,4,5-pentacarboxylic acid, mellitic acid, quinolinic acid, 1H-pyrazole-3,4-dicarboxylic acid, 1H-pyrazole-1,3,4-tricarboxylic acid, pyridine-2,4,5-tricarboxylic acid.

In some embodiments, the crosslinked polyamine particles are, comprise or consist essentially of, a hyperbranched polymer or residue thereof, a hyperbranched copolymer or residue thereof, a hyperbranched polymer network and/or a hyperbranched copolymer network or a pharmaceutical composition comprising the same.

In some embodiments, the hyperbranched polymers or copolymers may include polymers and or copolymers where from 10-95%, for example 10-75%, 25%-75%, 30%-60%, such as greater than 20%, 25%, 30%, 35%, 40%, 45%, 50%, or greater than 55% and less than 95% of the amine groups in the polymer or copolymer comprise secondary amine groups. In some embodiments, the hyperbranched polymers and/or copolymers include polymers and or copolymers where greater than 10% and less than 90%, for example, from 15%-85%, 20%-80%, 30%-70%, such as 35%, 40%, 45%, 50%, 55%, 60% or 65% of the non-terminal, non-amido amine groups in the polymer or copolymer are tertiary amines. In some embodiments, the hyperbranched polymers and/or copolymers may have a degree of branching of from 0.10 to 0.95, such as from 0.25-0.75, 0.30-0.60, or such as a degree of branching of 0.2, 0.25, 0.3, 0.35, 0.4, 0.45, 0.5, 0.55 which, in some embodiments may be calculated according to the following formula:

$$Degree of Branching = \frac{{N}_{p} + {N}_{t}}{{N}_{p} + {N}_{t} + {N}_{s}}$$

where

Nₚ=the number of primary amine units in the polymer (e.g.,

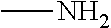

units);

Nₜ=the number of tertiary amine units in the polymer (e.g.,

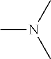

units; and

Nₛ=the number of secondary amine units in the polymer (e.g.,

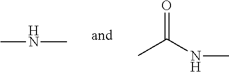

units).

In some embodiments, the hyperbranched polymers and/or copolymers have a polydispersity of greater than 1.2, for example greater than 1.3, 1.4, 1.5, 1.75, 2.0, 2.5 or even greater than 3.0, such as from 1.2-6, such as 1.5-5 or 2-4. In some embodiments, the hyperbranched polymers and/or copolymers may be branched and may be characterized by a plot of log (Mᵥ) versus log (η) that has no maximum, where Mᵥ represents the viscosity averaged molecular weight of the polymer or copolymer and η represents the intrinsic viscosity of the polymer or copolymer. For example, in some embodiments, the hyperbranched polymers and copolymers make the following equation is true: d(log(η))/d(log(Mᵥ))≠0.

In some embodiments, the hyperbranched polymers and/or copolymers may have random, variable length branching. For example, the hyperbranched polymers or copolymers may exhibit branching that does not conform to a regular or easily predictable or quantifiable pattern of occurrence or length and instead results from essentially random molecular interactions that may be driven by a wide variety of different variables such as, for example, monomer concentration, reactivity, pH, solvent, temperature, charge-charge interactions, catalysis, order of addition, and any other reaction parameters.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof is derived from or comprises a residue of a multi-amine monomer and a residue of a multifunctional monomer comprising two or more amine-reactive groups such as, for example, vinyl groups, carboxylic acid groups, ester groups, halogen groups, OSO₂R groups, or —C(O)R groups, where R independently represents a substituted or un-substituted C₁-C₂₀ alkyl radical such as a C₁, C₂, C₃, C₄, C₅ or C₆ radical, a substituted or un-substituted aryl radical, a substituted or un-substituted heteroaryl radical and/or combinations thereof and where the copolymer is hyperbranched.

The amine-reactive groups may react with the multi-amine via any suitable reaction, for example via a condensation or polycondensation reaction or via an alkylation reaction. In some embodiments, the hyperbranched polymer or residue thereof may be derived from a monomer comprising one or more amine groups and one or more multifunctional amine reactive groups, such as a multi-amine ester or a multi-amine multi-ester. In some embodiments, the reaction to form the polymer or copolymer may include a combination of different reactions, such as a combination of alkylation and condensation reactions. In some embodiments the reaction or reactions may be controlled by any suitable means including, for example, choice of solvent, temperature, concentration of reactants, protection using protecting groups, pH and/or any other suitable methods.

In some embodiments, the multi-amine monomer is selected from the multi-amines described elsewhere herein. In some embodiments, the multi-amine comprises from 2-20, such as 3, 4, 5, 6, 7, 8, 9, 10, 11, 12, 13, 14, 15, 16, 17, 18 or 19 terminal amine groups.

In some embodiments, the multifunctional monomer comprising two or more amine-reactive groups is selected from the group consisting of:

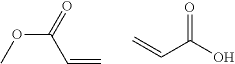

and combinations thereof.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof is derived from comonomers represented by the following Formulas XXXI and XXXII:

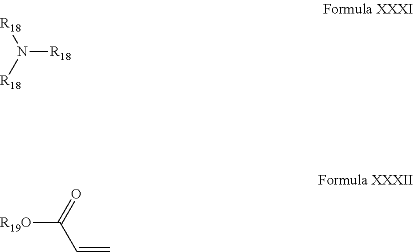

wherein R₁₈ independently represents a hydrogen radical, —RNH₂, —R—N—(R—NH₂)₂ or —R—N—(R—N—(R—NH₂)₂)₂, wherein R independently represents a branched or unbranched, substituted or unsubstituted alkyl radical for example a C₁ to C₂₀ alkyl radical, such as a C₁, C₂, C₃, C₄, C₅ or C₆ radical, with the proviso that at least one R₁₈ is not a hydrogen radical; R₁₉ independently represents a hydrogen radical or a branched or unbranched, substituted or unsubstituted alkyl radical for example a C₁ to C₂₀ alkyl radical, such as a C₁, C₂, C₃, C₄, C₅ or C₆ radical.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof is derived from comonomers represented by the following Formulas XXXI and XXXII:

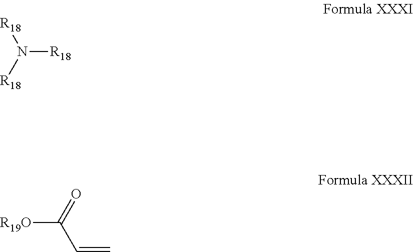

wherein R₁₈ independently represents a hydrogen radical, —RNH₂, —R—N—(R—NH₂)₂ or —R—N—(R—N—(R—NH₂)₂)₂, wherein R independently represents a branched or unbranched, substituted or unsubstituted alkyl radical for example a C₁ to C₂₀ alkyl radical, such as a C₁, C₂, C₃, C₄, C₅ or C₆ radical, with the proviso that at least one R₁₈ is not a hydrogen radical; R₁₉ independently represents a hydrogen radical or a branched or unbranched, substituted or unsubstituted alkyl radical for example a C₁ to C₂₀ alkyl radical, such as a C₁, C₂, C₃, C₄, C₅ or C₆ radical, where the copolymer is hyperbranched.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof comprises at least one multi-amine or residue thereof and at least one ester or multi-ester or residue thereof; where the copolymer is hyperbranched.

In some embodiments the multi-ester is selected from the group consisting of:

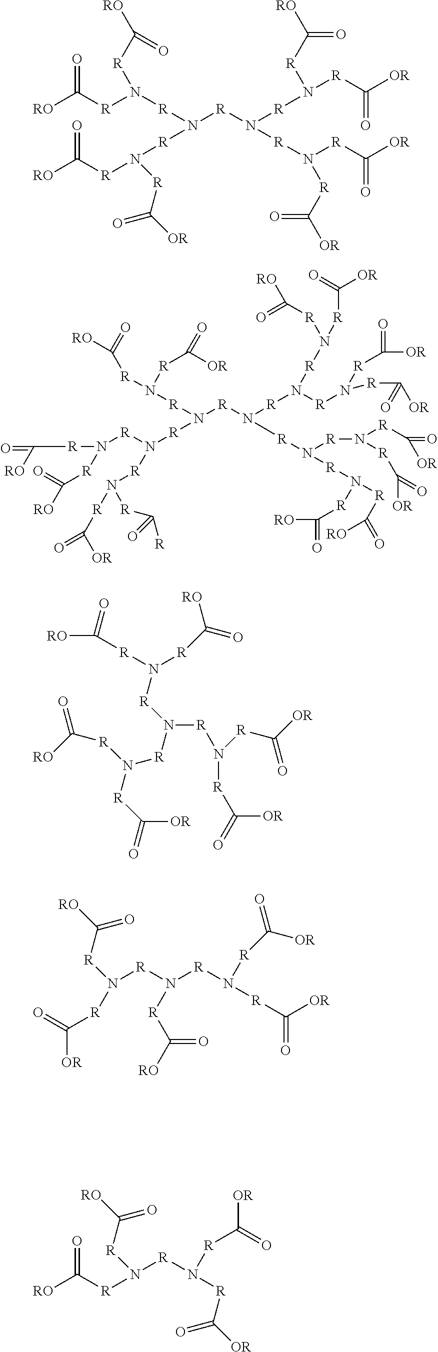

and combinations thereof, where R independently represents a branched or unbranched, substituted or unsubstituted alkyl radical for example a C₁ to C₂₀ alkyl radical, such as a C₁, C₂, C₃, C₄, C₅ or C₆ radical.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof comprises at least one compound or residue thereof, said compound represented by the following Formula XXXIII:

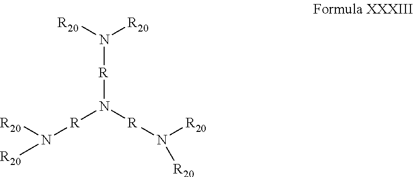

wherein R independently represents a branched or unbranched, substituted or unsubstituted alkyl radical for example a C₁ to C₂₀ alkyl radical, such as a C₁, C₂, C₃, C₄, C₅ or C₆ radical; R₂₀ independently represents a hydrogen radical or a unit independently represented by the following Formula XXXIV, with the proviso that at least one R₂₀ comprises a group represented by Formula XXXIV:

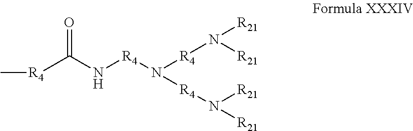

wherein R₄ independently represents

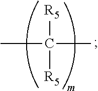

wherein m independently represents an integer from 1-20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound; R₂₁ represents a hydrogen radical or a unit according to Formula XXXIV and where the copolymer is hyperbranched.

In some embodiments the copolymer comprises one or more groups represented by the following Formula XXXV:

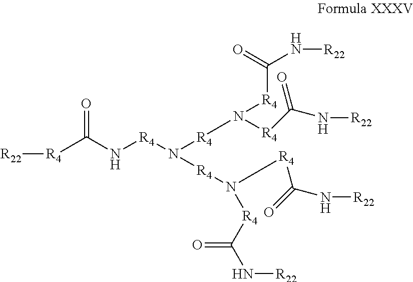

wherein R₂₂ comprises a link to a portion of a copolymer or copolymer network comprising a residue of a compound, said compound independently represented by Formula XXXIII.

In some embodiments, the present invention comprises crosslinked polyamine particles comprising or derived from a polymer or residue thereof, a pharmaceutical composition comprising crosslinked polyamine particles comprising or derived from said polymer or residue thereof or a method of using the same in a therapeutically effective amount to remove a compound or ion, such as a phosphorous-containing compound or a phosphorous-containing ion (e.g. phosphate), from the gastrointestinal tract of an animal where the polymer is derived from a monomer or comprises a residue of a monomer, where the monomer comprises an amine ester monomer having one or more amine reactive groups and one or more amine groups and where the polymer is hyperbranched.

In some embodiments the multi-amine ester monomer may be selected from the group consisting of

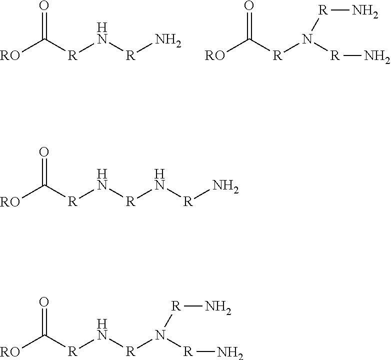

and combinations thereof.

In some embodiments, the present invention comprises crosslinked polyamine particles comprising or derived from a polymer or residue thereof, a pharmaceutical composition comprising crosslinked polyamine particles comprising or derived from said polymer or residue thereof or a method of using the same in a therapeutically effective amount to remove a compound or ion, such as a phosphorous-containing compound or a phosphorous-containing ion (e.g. phosphate), from the gastrointestinal tract of an animal where the polymer is derived from a monomer represented by the following Formula XXXVI:

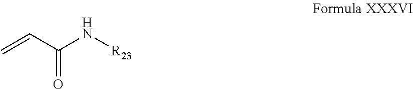

wherein R₂₃ represents —R—R₂₄, —R—N—(R₂₅)₂, —R—N(R—R₂₄)—R₂₅ or —R—N(R—R₂₄)—R—N(R₂₅)—R—N—(R₂₅)₂; R independently represents a branched or unbranched, substituted or unsubstituted alkyl radical, for example a C₁ to C₂₀ alkyl radical, such as a C₁, C₂, C₃, C₄, C₅ or C₆ radical; R₂₄ independently represents —NH₂ or NH₃⁺Cl⁻; R₂₅ independently represents —R—N—(R—R₂₄)₂ and where the polymer is hyperbranched.

In some embodiments the polymer comprises one or more groups represented by one or more of the following Formulas XXXVII-XXXVIII:

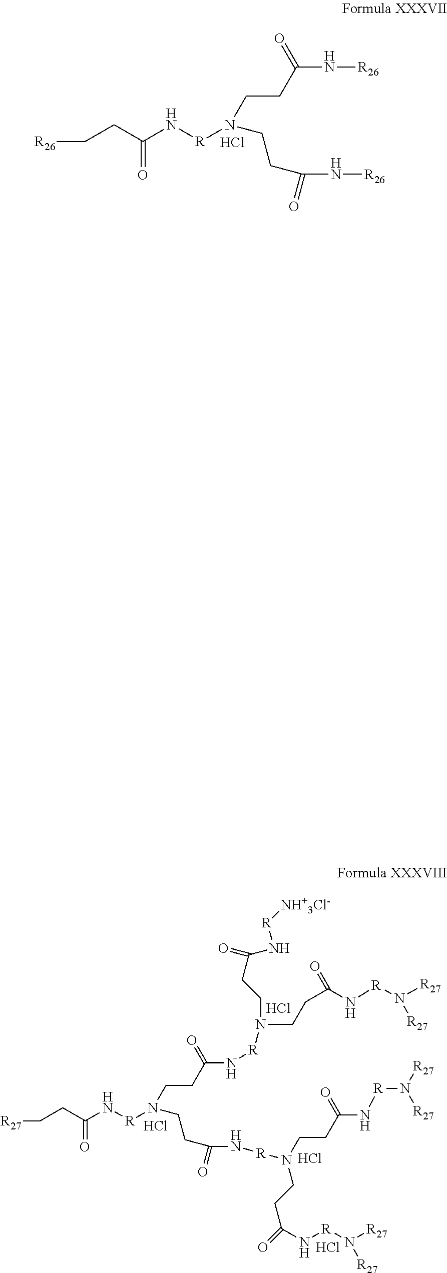

wherein R₂₆ and R₂₇ independently represent a link to a portion of a copolymer or comprising a residue of a monomer, said monomer independently represented by Formula XXXVI.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof comprises at least one amine compound or residue thereof, said amine compound represented by the following Formula XXXIX:

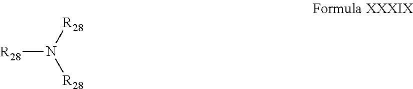

wherein R₁₁ independently represents a hydrogen radical or a unit independently represented by the following Formula XL, with the proviso that at least one R₂₈ comprises a group represented by Formula XL:

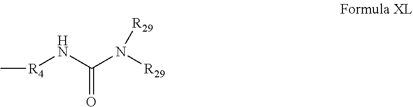

wherein R₄ independently represents

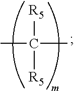

wherein m independently represents an integer from 1-20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound; R₂₉ represents a hydrogen radical or a unit according to Formula XL, where the copolymer is hyperbranched.

In some embodiments, the multifunctional monomer comprising two or more amine-reactive groups may comprise a multi-haloalkyl amine selected from the group consisting of:

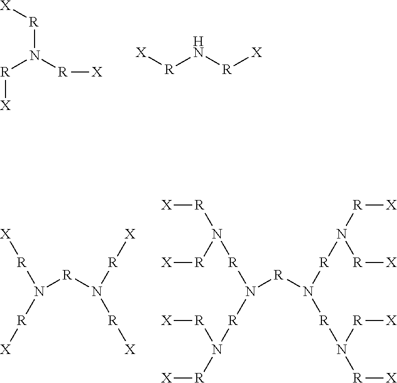

and combinations thereof, where R independently represents a branched or unbranched, substituted or un-substituted alkyl radical such as, for example a C₁ to C₂₀ alkyl radical such as a C₁, C₂, C₃, C₄, C₅, or C₆ alkyl radical and X independently represents —NH₂, —Cl, —Br, or —I, with the proviso that at least two X groups are not NH₂, such as, for example,

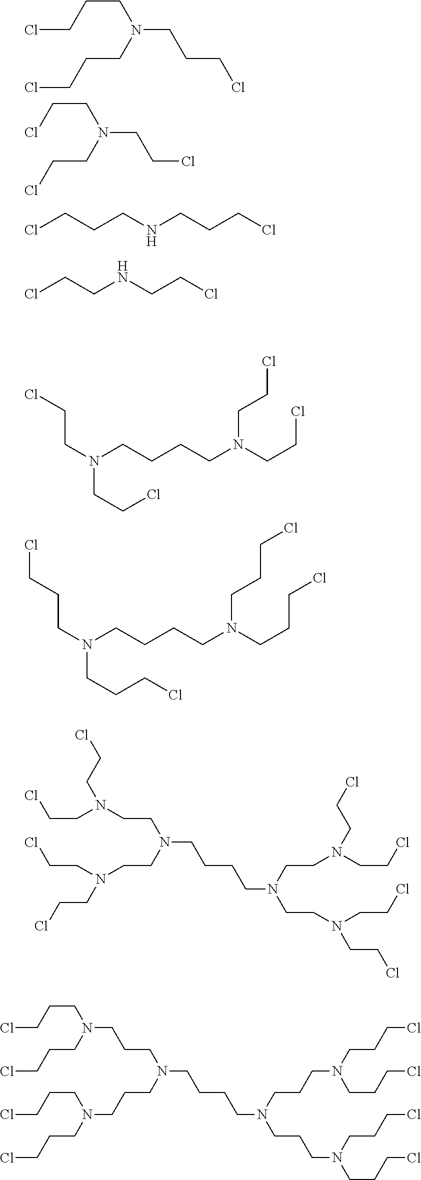

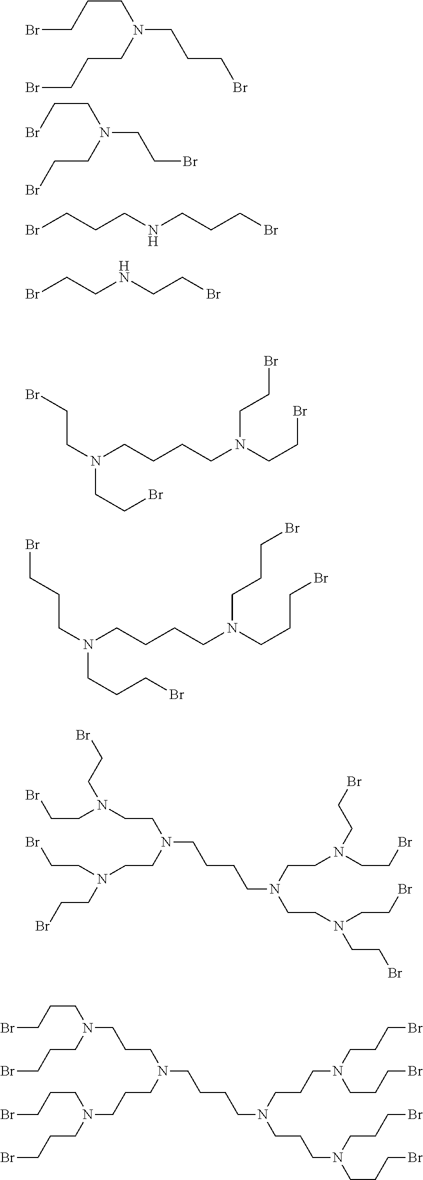

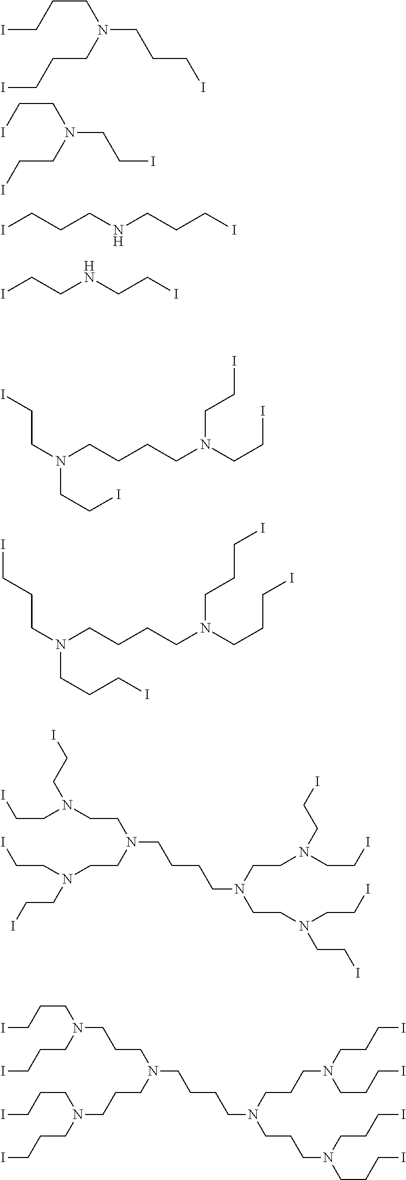

and combinations thereof.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof, where the copolymer is derived from comonomers represented by the following Formulas XLI and XLII:

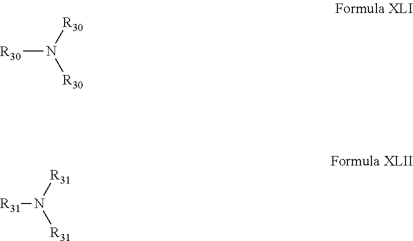

wherein R₃₀ independently represents a hydrogen radical, —R or —R—N(H)₂₋ₘ—(R—N(H)₂₋ₙ—(R—NH₂)ₙ)ₘ, or R₃₀ and another R₃₀ combine to form a heterocyclic ring, such as for example a heterocyclic ring comprising 1-4 heteroatoms, such as 1, 2, 3 or 4 heteroatoms, such as 1-4 nitrogen atoms, where the ring also includes 1-10 carbon atoms, such as 1, 2, 3, 4, 5, 6, 7, 8, or 9 carbon atoms; n and m independently represents an integer from 0 to 2, such as 0, 1 or 2, preferably either n or m is 1; R independently represents an oxygen radical, —CONR₃₂R₃₃, a branched or unbranched, substituted or un-substituted alkyl radical, for example a C₁ to C₂₀ alkyl radical such as a C₁, C₂, C₃, C₄, C₅, or C₆ alkyl radical, a branched or unbranched, substituted or un-substituted alkenyl radical, for example a C₂ to C₂₀ alkenyl radical such as a C₂, C₃, C₄, C₅, C₆, or C₇ alkenyl radical, a sulfur radical, or an SO₂ radical; R₃₂ and R₃₃ independently represent a hydrogen radical or a branched or unbranched, substituted or un-substituted alkyl radical, for example a C₁ to C₂₀ alkyl radical such as a C₁, C₂, C₃, C₄, C₅, or C₆ alkyl radical; R₃₁ independently represents a hydrogen radical, an electrophilic group (E) or —RE, with the proviso that at least one R₃₀ and at least one R₃₁ are not H, and where the copolymer is hyperbranched.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof, where the copolymer is derived from comonomers comprising at least one multi-amine or residue thereof and at least one multi-haloalkyl amine or residue thereof, and where the copolymer is hyperbranched.

In some embodiments, the present invention comprises crosslinked polyamine particles comprising or derived from a polymer or residue thereof or a copolymer or residue thereof, a pharmaceutical composition comprising crosslinked polyamine particles comprising or derived from said polymer or residue thereof or said copolymer or residue thereof thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof, where the polymer or copolymer comprises one or more groups represented by one or more of the following Formulas XLIII-XLV:

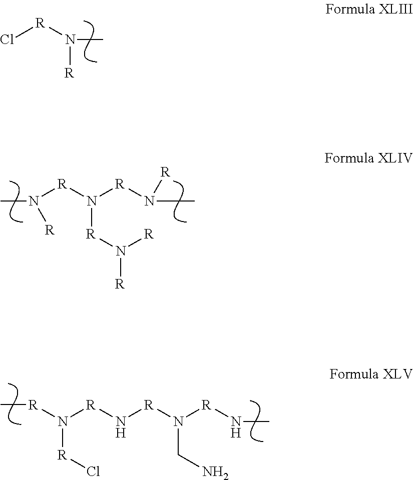

wherein R independently represents a branched or unbranched, substituted or un-substituted alkyl radical such as, for example a C₁ to C₂₀ alkyl radical such as a C₁, C₂, C₃, C₄, C₅, or C₆ alkyl radical, where the polymer or copolymer.

In some embodiments, the multifunctional monomer comprising two or more amine-reactive groups may comprise a multifunctional sulfonyl-containing monomer comprising two or more amine-reactive groups. In some embodiments, the multifunctional sulfonyl-containing monomer comprising two or more amine-reactive groups may be selected from the group consisting of:

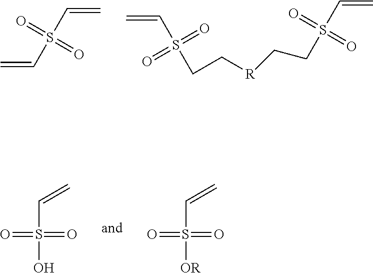

wherein R independently represents a branched or unbranched, substituted or unsubstituted alkyl or aryl radical.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof, where the copolymer is derived from comonomers represented by the following Formulas XLVI and XLVII:

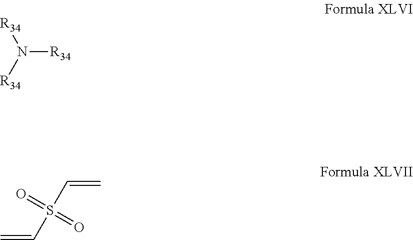

where R₃₄ independently represents a hydrogen radical, —R or —R—N(H)₂₋ₘ—(R—N(H)₂₋ₙ—(R—NH₂)ₙ)ₘ or R₃₄ and another R₃₄ combine to form a heterocyclic ring, such as for example a heterocyclic ring comprising 1-4 heteroatoms, such as 1, 2, 3 or 4 heteroatoms, such as 1-4 nitrogen atoms, where the ring also includes 1-10 carbon atoms, such as 1, 2, 3, 4, 5, 6, 7, 8, or 9 carbon atoms; n and m independently represents an integer from 0 to 2, such as 0, 1 or 2, preferably either n or m is 1; R independently represents a branched or unbranched, substituted or unsubstituted alkyl radical, for example a C₁ to C₂₀ radical such as a C₁, C₂, C₃, C₄, C₅, or C₆ radical, with the proviso that at least one R₃₄ is not a hydrogen radical or —R and where the copolymer is hyperbranched.

In some embodiments, the present invention comprises crosslinked polyamine particles comprising or derived from a copolymer or residue thereof, a pharmaceutical composition comprising crosslinked polyamine particles comprising or derived from said copolymer or residue thereof or a method of using the same in a therapeutically effective amount to remove a compound or ion, such as a phosphorous-containing compound or a phosphorous-containing ion (e.g. phosphate), from the gastrointestinal tract of an animal where the copolymer or residue thereof, where the copolymer where the copolymer comprises a residue of one or more multi-amine compounds and a residue of one or more vinyl sulfonyl-containing compounds. In some embodiments, the multi-amine monomer comprises at least one secondary amine.

In some embodiments, the invention is a method of treating a phosphate imbalance disorder such as hyperphosphatemia comprising administering a therapeutically effective amount of crosslinked polyamine particles comprising or derived from one or more polymers or copolymers of the invention.

### Polymerization

In some embodiments, the crosslinked polyamine particles according to the invention may comprise dendrimers which may be formed from any suitable reaction scheme. Dendrimers are macromolecular compounds that comprise a core that includes functional groups and dendritic branches that may be formed through a series of iterative reaction sequences with the functional groups on the core to form a branched macromolecule. In some embodiments the reactive functional groups comprise hydroxyl groups and/or amine groups. The functional groups will have functionalities that are dependent on the type of group. For example, hydroxyl groups have a functionality of one, while primary amines generally have a functionality of 2, though they may be quaternized. In some embodiments, dendrimers of the present invention are prepared by a Michael addition of a substituted or un-substituted α, β unsaturated nitrile to one or more of the functional groups on a core to replace a hydrogen in the functional group on the core with a nitrile group. The nitriles of the nitrile groups of the resulting compound are then chemically reduced, for example via hydrogenation, to form the corresponding primary amines. The Michael addition and subsequent reduction may be repeated on the primary amines generally yielding a branched tertiary amine. Subsequent Michael additions and reductions may be repeated one or more times to provide the branched structure characteristic of dendrimers. A schematic of this process is provided below in Scheme I, using acrylonitrile as the substituted or un-substituted α, β unsaturated nitrile and mannitol as the core:

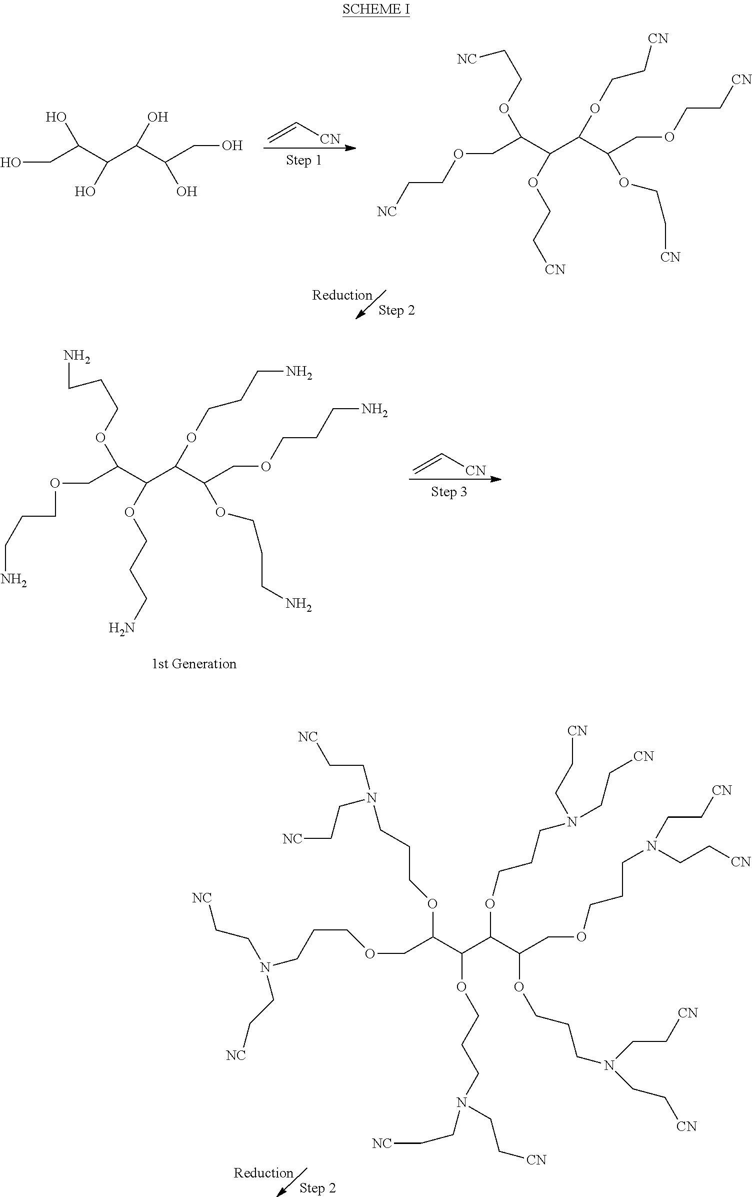

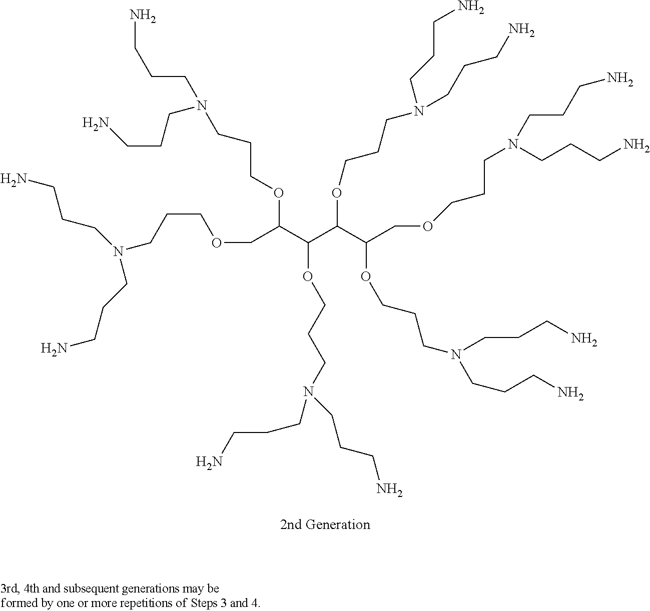

In some embodiments, each iteration of Michael addition and subsequent reduction may be considered one generation. Thus, for some embodiments, a compound having one generation of dendritic branching may have undergone one iteration of Michael addition and reduction, compounds having two generations of dendritic branching may have undergone two iterations of Michael addition and reduction, compounds having three generations of dendritic branching may have undergone three iterations of Michael addition and reduction, compounds having four generations of dendritic branching may have undergone four iterations of Michael addition and reduction, etc. Generally dendrimers according to some embodiments of the present invention may have from 1-10, such as 2, 3, 4, 5, 6, 7, 8, or 9 generations of dendritic branching.

In some embodiments, a method of making hyperbranched polymers and copolymers of the invention can include any suitable method such as addition of a multi-amine to a multifunctional monomer comprising two or more amine-reactive groups in a reactor and heating the mixture. In some embodiments the mixture may be heated to greater than 25° C., for example 30° C., 35° C., 37° C., 40° C., 45° C., 50° C. or higher. In some embodiments, the mixture may be heated from 1 hour to several days, such as 1-7 days, such as from 2-6 days or 24, 48, 72 or 96 hours. The resulting polymer or copolymer may be purified using any suitable method, such as precipitation and washing, or dialyzation. The copolymer may then be dried under vacuum or lyophilized to yield the desired copolymer.

The copolymer prepared above may then be subsequently crosslinked using any suitable method. For example, the copolymer may be mixed with a crosslinking agent, such as for example epichlorohydrin, in a suitable solvent, such as, for example, water and stirred. In some embodiments, the crosslinking agent may be added in one or more aliquots such as 1-10 aliquots, such as 2-8 or 3-5 aliquots. In some embodiments, the solution may be stirred and heated for 1 hour to 5 days, such as 1, 2, 3, 4 or 5 days. A gel may form and may be cured, broken, resuspended and washed one or more times and then dried, such as in a forced air oven or via lyophilization. In some embodiments, washing may include adjustment of the pH of the material.

In some embodiments, the polymers or copolymers may be crosslinked in a bulk solution (i.e. using the neat amine polymer and neat crosslinking agents) or in dispersed media. When a bulk process is used, solvents are selected so that they co-dissolve the reactants and do not interfere with the crosslinking reaction. Suitable solvents include water, low boiling alcohols (methanol, ethanol, butanol), acetonitrile, dimethylformamide, dimethylsulfoxide, acetone, methylethylketone, and the like.

Other polymerization methods may include a single polymerization reaction, stepwise addition of individual monomers via a series of reactions, the stepwise addition of blocks of monomers, combinations of the foregoing, or any other method of polymerization, such as, for example, direct or inverse suspension, condensation, phase transfer, emulsion, precipitation techniques, polymerization in aerosol or using bulk polymerization/crosslinking methods and size control processes such as extrusion and grinding. Processes can be carried out as batch, semi-continuous and continuous processes. For processes in dispersed media, the continuous phase can be selected from apolar solvents such as toluene, benzene, hydrocarbon, halogenated solvents, supercritical carbon dioxide, and the like. With a direct suspension process, water can be used, although salt brines are also useful to “salt out” the amine and crosslinking agents in a droplet separate phase.

Examples of some suitable polymerization methods may be found, for example, in the following patents and patent applications each of which is incorporated herein by reference in their entirety: U.S. Pat. No. 4,605,701; U.S. Pat. No. 5,496,545; U.S. Pat. No. 5,618,530; U.S. Pat. No. 5,679,717; U.S. Pat. No. 5,693,675; U.S. Pat. No. 5,702,696; US WO 96/21454; WO 98/57652; EP 7372352; DE 4227019.

A non-limiting example of a preparation of a crosslinked polyamine may occur as follows: a polymer or copolymer prepared as previously described herein may be neutralized if necessary, with a base, such as ammonium hydroxide or NaOH After the optional neutralization, the polymer or copolymer may be emulsified with a crosslinking agent, such as epichlorohydrin using a static or high shear mixer. The resulting oil-in-water emulsion may be polymerized using batch reactor or a single screw or twin screw kneading or LIST reactor. The temperature, polymer or copolymer concentration, ratio of polymer or copolymer to crosslinking agent, rotor speed, and/or work supplied to the reacting polymer or copolymer may be controlled to help achieve the desired particle size. The polymer or copolymer leaving the reactor may be suspended in a solvent, such as water, ethanol, ethanol/water mixtures, isopropanol, isopropanol/water mixtures and mixtures thereof followed by filtering and optionally re-suspending one or multiple times, may be milled, wet milled, neutralized and/or protonated using a suitable source such as HCl, CO₂ or carbonic acid, may be milled and/or may be separated before drying using centrifugal force, such as using hydrocyclones or centrifuges. The polymer or copolymer may be dried using any suitable method such as using a convection oven, a vacuum oven or a fluidized bed and then may be ground, milled and/or sieved or fractionated to a particular desired mesh or particle size after drying. Alternatively, when a solvent that comprises ethanol, ethanol/water mixtures, isopropanol or isopropanol/water mixtures is used, the polymer may not need to be dried prior to grinding, milling and/or sieving or fractionating. In some embodiments, the solvent is water and the polymer or copolymer is dried prior to grinding.

In some embodiments, the crosslinking reaction can be run in any suitable vessel or reactor and may be run batch-wise or in a continuous fashion. In some embodiments, the crosslinking reaction is run in a reactor designed for high viscosity processing which has agitation means capable of mixing the reactants prior to gelation and breaking the gel into small pieces or crumb after gelation. Examples of such reactors are LIST reactors, such as the LIST-DISCOTHERM B manufactured by LIST Inc. LIST reactors may be supplied for batch or continuous operation and are particularly useful for thermal processes such as drying or reactions, where mixing or kneading is necessary to process viscous, pasty, crusting or gelatinous materials such as cross-linked polyamine polymer. In some embodiments, such a reactor may include a horizontal, cylindrical housing, and a concentric agitator shaft with disc elements perpendicular to the axis carrying peripheral mixing/kneading bars. Stationary hook-shaped bars may be set in the shell and may interact with, and clean, the shaft and disk elements as they rotate. Shell, shaft, and disc elements, all of which contribute to heat transfer can be heated or cooled. The unit generally operates with a fill level of 60 to 75 percent reactor capacity. Typical shaft speeds range from 5 to 100 rotations per minute (“rpm”) with high installed torque. The combined effect of the intensive mixing and kneading action and the self cleaning of the heat exchange surfaces results in high heat and mass transfer rates. In batch units the mixing bars may be arranged to perform optional mixing. For continuous operation, the arrangement of the internal geometry provides a forward plug flow movement of the material. However, the axial conveying rate is nearly independent of agitator rotation speed, making it possible to operate at high agitator rotation speeds optimizing heat and mass transfer. Furthermore, the positioning of the disc elements enables the processing of liquid feed stocks directly through to a solid free flowing material without recycling of dry product. The unique design of the LIST reactor eliminates the formation of a single, continuous, congealed mass. As gelation occurs, the self-wiping concentric agitator shaft and disc elements create easy to handle clumps of gel.

In some embodiments, after crosslinking, the polymer may be hydrated and/or suspended in water, stirred until a gel forms and allowed to cure for a period of time, such as from 30 minutes to 30 hours, from 1 hour to 29 hours, from 3 hours to 28 hours, from 6 hours to 27 hours, from 9 hours to 26 hours, from 12-25 hours, such as 15-21 hours or 17-19 hours. After curing, the gelled polymer may be broken into pieces using any suitable instrument, diluted with water and/or wet milled to a desired constituent particle size. The wet milling may use any known wet milling method and may include using a blender or homogenizer. In some embodiments, after wet milling, or after curing, the gel may be neutralized and/or washed multiple times until the gel (in suspension) has a conductivity of approximately 1 mS/cm³ or less. The polymer or copolymer may then be protonated, for example carbonated using dry ice, CO₂ and/or carbonic acid or any other suitable carbonating system. After protonation, the gel may be dried using any suitable method such as using a convection oven, a vacuum oven and/or a fluidized bed and then may be ground, milled and/or sieved or fractionated to a particular desired particle or mesh size after drying. Alternatively, when a solvent that comprises ethanol, ethanol/water mixtures, isopropanol or isopropanol/water mixtures is used to wash the gel before or after carbonation, it may not be necessary to dry the gel prior to grinding, milling and/or sieving or fractionating. In some embodiments, the solvent is water and the polymer or copolymer is dried prior to grinding.

In some embodiments, crosslinked polyamine polymers or copolymers of the invention may be formed from constituent particles of the crosslinked polyamine, which may be placed in a solvent, such as such as water, ethanol, ethanol/water mixtures, isopropanol, isopropanol/water mixtures and mixtures thereof, dried using any suitable method such as using a convection oven, a vacuum oven or a fluidized bed, and then ground, milled and/or sieved or fractionated to a particular desired particle or mesh size after drying. Alternatively, when a solvent that comprises ethanol, ethanol/water mixtures, isopropanol or isopropanol/water mixtures is used to wash the gel before or after carbonation, it may not be necessary to dry the gel prior to grinding, milling and/or sieving or fractionating. In some embodiments, the solvent is water and the polymer or copolymer is dried prior to grinding.

In some embodiments, crosslinked polyamine polymers of the invention may be formed using or starting from epichlorohydrin crosslinked constituent particles of one of the polymers of copolymers described herein. In some embodiments, the constituent particles range may be suspended in a solvent such as water, ethanol, ethanol/water mixtures, isopropanol, isopropanol/water mixtures and mixtures thereof, stirred until forming a gel and then cured for from 30 minutes to 30 hours, such as from 1 hour to 29 hours, from 3 hours to 28 hours, from 6 hours to 27 hours, from 9 hours to 26 hours, from 12-25 hours, such as 15-21 hours or 17-19 hours. The gel may then be dried for from 30 minutes to 30 hours, such as from 1 hour to 29 hours, from 3 hours to 28 hours, from 6 hours to 27 hours, from 9 hours to 26 hours, from 12-25 hours, such as 15-21 hours or 17-19 hours and the dried gel may then be milled using any suitable milling or grinding equipment and sieved or fractionated to the desired particle size/particle size distribution. A solvent that comprises water, ethanol, ethanol/water mixtures, isopropanol or isopropanol/water mixtures may be used to wash the gel after curing and it may not be necessary to dry the gel prior to grinding, milling and/or sieving or fractionating. In some embodiments, the solvent is water and the polymer or copolymer is dried prior to grinding.

In some embodiments, the solvent comprises water. In some embodiments, the solvent comprises an ethanol/water mixture such as from 5 wt. % to 95 wt. % ethanol and from 5 wt. % to 95 wt. % water. In some embodiments, the solvent comprises an isopropanol/water mixture such as from 5 wt. % to 95 wt. % isopropanol and from 5 wt. % to 95 wt. % water.

In some embodiments, the gel may be cured at room temperature. In other embodiments, the gel may be cured at an elevated temperature such as from 30° C. to 65° C. In some embodiments, the gel may be dried in a forced air oven. In other embodiments, the gel may be dried in a vacuum oven. In other embodiments, the gel may be dried in a fluidized bed. Any suitable drying temperature may be used. In some embodiments, the drying temperature may be from 15° C. to 75° C., such as from 20° C. to 75° C., from 25° C. to 70° C. from 30° C. to 70° C., 35° C. to 65° C., from 40° C. to 65° C., from 45° C. to 60° C. or from 50° C. to 60° C.

In some embodiments, the polymer or copolymer or polymer or copolymer gel may be ground, wet milled and/or milled. Any suitable grinding or milling equipment may be used including manual grinding techniques such as mortar and pestle, potato or other mashers and automated grinding or milling using equipment such as blenders, grinders and mills including coffee grinders, industrial or other commercial blenders. In some embodiments, the polymer or copolymer or polymer or copolymer gel may be milled or ground using a jet-mill, a fluidized jet-mill, a pin-mill, a cosmomizer, a cavitation-mill and/or a dispersion mill. Examples of some suitable milling techniques may be found in Lachman et al, The Theory and Practice of Industrial Pharmacy (1986), the entire contents of which is hereby incorporated by reference. In some embodiments, the grinding or milling may be conducted in the presence of various grinding media that may assist in the grinding.

Any suitable method of controlling or achieving the desired particle size may be used. The particle size of the crosslinked polyamine polymers or copolymers may be controlled by controlling various polymerization and crosslinking process parameters such as temperature, monomer and crosslinker concentration, solvent, monomer to solvent ratio, polymer or copolymer to solvent ratio, pH, infusion rate, mixing rate, and by selecting the downstream process and processing parameters. For example, the particle size may be affected by the orifice size of a spray dryer nozzle and the height of a spray drying tower or the drying temperature. In addition, after crosslinking, the particles may be further processed to achieve the desired particle size such as ground using a grinder or a mill or selectively sieved. Specific suitable downstream processing methods include, but are not limited to grinding, milling, wet milling, spray drying, sieving, precipitation, suspension or re-suspension and filtration, separation using passive or active centrifugal forces, spray-freezing and any combination thereof.

In some embodiments, the crosslinked polyamine particles may be formed using a ratio of (amine compound/monomer+crosslinker): solvent of between about 10:1 to about 1:10 (w/w), such as between about 7:1 to about 1:7 (w/w), between about 5:1 to about 1:5 (w/w), between about 4:1 to about 1:4 (w/w), between about 3.5:1 to about 1:3.5 (w/w), between about 3:1 to about 1:3 (w/w), between about 2.5:1 to about 1:2.5 (w/w), between about 2:1 to about 1:2 (w/w), or between about 1.5:1 to about 1:1.5 (w/w).

In some embodiments, crosslinked polyamine particles of the invention may not dissolve in solvents, and, at most, swell in solvents. The swelling ratio may be calculated according to the procedure in the Test Methods section below and is typically in the range of about 1 to about 150, such as about 2.5 to about 150, about 5 to about 150, about 5 to about 100, about 5 to about 80, about 5 to about 60, about 5 to about 40, or about 5 to about 20; for example, 1 to 20, 2.5 to 19, 5 to 18, 5 to 16 or 5 to 15, such as greater than 1 and less than 50, greater than 2.5 and less than 45, greater than 5 and less than 40, greater than 5 and less than 20, greater than 9 and less than 20, greater than 11 and less than 20, such as 1, 2, 3, 4, 5, 6, 7, 8, 9, 10, 11, 12, 13, 14, 15, 16, 17, 18, 19 or more.

Crosslinking agents are typically compounds having at least two functional groups that are selected from a halogen group, carbonyl group, epoxy group, ester group, acid anhydride group, acid halide group, isocyanate group, vinyl group, and chloroformate group. The crosslinking agent may be attached to the carbon backbone or to a nitrogen of an amine polymer, amine monomer or residue thereof.

Examples of crosslinking agents that are suitable for synthesis of the crosslinked polyamine particles of the present invention include, but are not limited to, one or more multifunctional crosslinking agents such as: dihaloalkanes, haloalkyloxiranes, alkyloxirane sulfonates, di(haloalkyl)amines, tri(haloalkyl)amines, diepoxides, triepoxides, tetraepoxides, bis(halomethyl)benzenes, tri(halomethyl)benzenes, tetra(halomethyl)benzenes, epihalohydrins such as epichlorohydrin and epibromohydrin, poly(epichlorohydrin), (iodomethyl)oxirane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, methyl acrylate and the like. When the crosslinking agent is an alkylhalide compound, a base may be used to scavenge the acid formed during the reaction. Inorganic or organic bases are suitable. NaOH is preferred. The base to crosslinking agent ratio may be between about 0.5 to about 2.

In some embodiments, the crosslinking agents may be used in the crosslinking reaction in an amount of from 7 wt. % to 70 wt %, such as from about 8 wt. % to about 65 wt. %, about 10 wt. % to about 65 wt. %, about 15 wt. % to about 60 wt. %, about 20 wt. % to about 60 wt. %, about 25 wt. % to about 60 wt. %, about 30 wt. % to about 60 wt. %, about 35 wt. % to about 55 wt. %, about 40 wt. % to about 55 wt. % or about 45 wt. % to about 55 wt. %. In some embodiments, the crosslinking agents may be used in the crosslinking reaction an amount of from about 8 wt. % to 11 wt. %, from about 9 wt. % to about 10.4 wt. % or from about 9.4 wt. % to about 10.2 wt. %, such as 8, 9, 9.4, 9.8 or 10 wt. %.

In some embodiments, the weight averaged molecular weight of the polymers and copolymers may be typically at least about 1000. For example, the molecular weight may be from about 1000 to about 1,000,000, such as about 2000 to about 750,000, about 3000 to about 500,000, about 5000 to about 250,000, about 10000 to about 100,000, such as from 15,000-80,000, 20,000 to 75,000, 25,000 to 60,000, 30,000 to 50,000, or 40,000 to 45,000.

The crosslinked polyamine polymers of some embodiments may be formed using a polymerization initiator. Generally, any initiator may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′-azobis(isobutyronitrile), 2,2′-azobis(N,N′-dimethyleneisobutyramidine)dihydrochloride, 2,2′-azobis(2-amidinopropane)dihydrochloride, 2,2′-azobis(N,N′-dimethyleneisobutyramidine), 1,1′-azobis(1-cyclohexanecarbo-nitrile), 4,4′-azobis(4-cyanopentanoic acid), 2,2′-azobis(isobutyramide) dihydrate, 2,2′-azobis(2-methylpropane), 2,2′-azobis(2-methylbutyronitrile), VAZO 67, cyanopentanoic acid, the peroxy pivalates, dodecylbenzene peroxide, benzoyl peroxide, di-t-butyl hydroperoxide, t-butyl peracetate, acetyl peroxide, dicumyl peroxide, cumyl hydroperoxide, dimethyl bis(butylperoxy) hexane.

In some embodiments, any of the nitrogen atoms within the crosslinked polyamine particles according to embodiments of the invention may optionally be quaternized to yield the corresponding positively charged tertiary nitrogen group, such as for example, an ammonium or substituted ammonium group. Any one or more of the nitrogen atoms in the crosslinked polyamines may be quaternized and such quaternization, when present, is not limited to or required to include terminal amine nitrogen atoms. In some embodiments, this quaternization may result in additional network formation and may be the result of addition of crosslinking, linking or amine reactive groups to the nitrogen. The ammonium groups may be associated with a pharmaceutically acceptable counterion.

In some embodiments, crosslinked polyamine particles of the invention may be partially or fully quaternized, including protonated, and may have a pharmaceutically acceptable counterion, which may be organic ions, inorganic ions, or a combination thereof. Examples of some suitable inorganic ions include halides (e.g., chloride, bromide or iodide) carbonates, bicarbonates, sulfates, bisulfates, hydroxides, nitrates, persulfates and sulfites. Examples of some suitable organic ions include acetates, ascorbates, benzoates, citrates, dihydrogen citrates, hydrogen citrates, oxalates, succinates, tartrates, taurocholates, glycocholates, and cholates. Preferred counterions include chlorides and carbonates.

In some embodiments, crosslinked polyamine particles of the invention may be protonated such that the fraction of protonated nitrogen atoms is from 1% to 100%, such as 10% to 75%, 20% to 60%, 25%% to 55%, 30% to 50%, 35% to 45% or about 40%.

In one embodiment, the pharmaceutically acceptable crosslinked polyamine particles are in partially or fully protonated form and comprise a carbonate anion. In one embodiment, the pharmaceutically acceptable crosslinked polyamine particles are in partially or fully protonated form and comprise a mixture of carbonate and bicarbonate counterions.

In some embodiments, crosslinked polyamine particles of the invention are characterized by their ability to bind compounds or ions. Preferably the crosslinked polyamine particles of the invention bind anions, more preferably they bind organophosphates, phosphate and/or oxalate, and most preferably they bind phosphate. For illustration, anion-binding crosslinked polyamine particles and especially organophosphate or phosphate-binding crosslinked polyamine particles will be described; however, it is understood that this description applies equally, with appropriate modifications that will be apparent to those of skill in the art, to other ions, compounds and solutes. While not wishing to be bound by any theory, crosslinked polyamine particles are believed to bind an ion, e.g., an anion, when they associate with the ion, generally though not necessarily in a noncovalent manner, with sufficient association strength that at least a portion of the ion remains bound under the in vitro or in vivo conditions in which the polymer is used for sufficient time to effect a removal of the ion from solution or from the body. A target ion may be an ion to which the crosslinked polyamine particles bind, and usually refers to the ion whose binding to the crosslinked polyamine particles is thought to produce the therapeutic effect of the crosslinked polyamine particles and may be an anion or a cation. Crosslinked polyamine particles of the invention may have more than one target ion.

For example, some of the crosslinked polyamine particles described herein exhibit organophosphate or phosphate binding properties. Phosphate binding capacity is a measure of the amount of phosphate ion a phosphate binder can bind in a given solution. Some embodiments of the crosslinked polyamine particles of the invention have an in vitro non-competitive phosphate binding capacity which is greater than about 0.2, 0.4, 0.5, 1.0, 1.2, 1.3, 1.4, 1.5, 1.6, 1.7, 1.8, 2.0, 2.5, 3.0, 3.5, 4.0, 4.5, 5.0, 6.0, 8.0, 10.0, greater than about 12, or up to about 14, mmol/g. In some embodiments, the in vitro non-competitive phosphate binding capacity of crosslinked polyamine particles of the invention is greater than about 0.4 mmol/g, greater than about 2.5 mmol/g, greater than about 3 mmol/g, greater than about 4.5 mmol/g or greater than about 6 mmol/g. In some embodiments, the in vitro non-competitive phosphate binding capacity can be between about 0.2 mmol/g and about 14 mmol/g, such as between about 0.4 mmol/g and about 10 mmol/g, between about 1.0 mmol/g and about 8 mmol/g, between about 1.5 mmol/g and about 8 mmol/g, between about 2.0 mmol/g and about 8 mmol/g, between about 2.5 mmol/g and about 8 mmol/g, between about 3 mmol/g and about 6 mmol/g or between about 3 mmol/g and about 5 mmol/g. The in vitro non-competitive phosphate binding capacity may be measured according to the techniques described in the Test Methods section below.

In some embodiments, the crosslinked polyamine particles according to the invention have an in vitro competitive phosphate binding capacity of between 0.4 mmol/g and 10 mmol/g, for example between 0.5 mmol/g and 7 mmol/g, between 0.6 mmol/g and 5 mmol/g, between 0.7 mmol/g and 4 mmol/g or between 0.8 mmol/g and 2.5 mmol/g throughout a physiologically significant time period. A physiologically significant time period may be the length of time during which significant uptake of a target ion occurs in a human. For example, for phosphate the physiologically significant time period may be from 0 to 5 hours, such as 0.5 to 5 hours, 1 to 4.5 hours, 1.5 to 4 hours, 2 to 3.5 hours or 1, 1.5, 2, 2.5, 3, 3.5, 4, 4.5 or 5 hours. The in vitro competitive phosphate binding capacity may be measured according to the techniques described in the Test Methods section below.

In some embodiments, the crosslinked polyamine particles of the present invention have an in vitro non-competitive phosphate binding capacity at 5 hours that is within 20%, for example within 15%, 12.5%, 10% or even 5% of that of sevelamer hydrochloride.

In some embodiments, the crosslinked polyamine particles according to the invention may have an in vitro competitive phosphate binging capacity at 60 minutes that is greater than 1.2 mmol phosphate/g of polymer, such as greater than 1.25 mmol/g, greater than 1.30 mmol/g, greater than 1.35 mmol/g, greater than 1.4 mmol/g, greater than 1.5 mmol/g, greater than 1.6 mmol/g, greater than 1.7 mmol/g, greater than 1.8 mmol/g, greater than 1.9 mmol/g or greater than 2.0 mmol/g. In some embodiments, the crosslinked polyamine particles according to the invention may have an in vitro competitive phosphate binging capacity at 60 minutes that is between 1.2 mmol/g and 10 mmol/g, such as between 1.2 mmol/g and 7.5 mmol/g, between 1.2 mmol/g and 5.0 mmol/g, between 1.2 mmol/g and 4.0 mmol/g, between 1.25 mmol/g and 4.0 mmol/g, between 1.3 mmol/g and 4.0 mmol/g, between 1.35 mmol/g and 4.0 mmol/g, between 1.4 mmol/g and 4.0 mmol/g, between 1.5 mmol/g and 4.0 mmol/g, between 1.6 mmol/g and 4.0 mmol/g, between 1.7 mmol/g and 4.0 mmol/g, or between 1.8 mmol/g and 4.0 mmol/g.

In some embodiments, the crosslinked polyamine particles of the present invention have an in vitro competitive phosphate binding capacity at 1 hour of greater than 20%, for example greater than 30%, greater than 35%, greater than 40% or greater than 45% of the 5 hour or 300 minute in vitro non-competitive phosphate binding capacity of said polymer.

In some embodiments, the crosslinked polyamine particles of the invention have an in vivo phosphate binding capacity of between 0.2 mmol/g and 14 mmol/g, such as between 0.3 mmol/g and 14 mmol/g, between 0.4 mmol/g and 12.5 mmol/g, between 0.5 mmol/g and 10 mmol/g, between 0.75 mmol/g and 8 mmol/g, between 1.0 mmol/g and 6 mmol/g, between 1.25 mmol/g and 5 mmol/g, between 1.5 mmol/g and 4.5 mmol/g, between 2.0 mmol/g and 4.0 mmol/g or between 2.5 mmol/g and 3.5 mmol/g. The in vivo phosphate binding capacity may be measured in any animal, such as any mammal, such as humans or rats. The test methods detail a procedure for measuring the in vivo phosphate binding capacity in rats, which may be suitably modified as appropriate for measurement in humans.

In some embodiments, the crosslinked polyamine particles of the invention have an in vitro bile acid binding capacity of between 0.5 mmol/g and 14 mmol/g, such as between 0.3 mmol/g and 14 mmol/g, between 0.4 mmol/g and 12.5 mmol/g, between 0.5 mmol/g and 10 mmol/g, between 0.75 mmol/g and 8 mmol/g, between 1.0 mmol/g and 6 mmol/g, between 1.25 mmol/g and 6 mmol/g, between 1.5 mmol/g and 6 mmol/g, between 2.0 mmol/g and 6 mmol/g or between 2.5 mmol/g and 6 mmol/g, such as greater than 1.00, 1.5, 2.0, 2.5, 3.0, 3.5, 4, 4.5, 5.0, 5.5, 6.0, 7.0, 8.0, 9.0, 10.0, 11.0, 12.0 or greater than 13.0 mmol/g. The in vitro bile acid binding capacity may be determined according to the procedure detailed in the Test Procedures.

In some embodiments, the crosslinked polyamine particles of the invention have an in vivo bile acid binding capacity of between 0.5 mmol/g and 14 mmol/g, such as between 0.3 mmol/g and 14 mmol/g, between 0.4 mmol/g and 12.5 mmol/g, between 0.5 mmol/g and 10 mmol/g, between 0.75 mmol/g and 8 mmol/g, between 1.0 mmol/g and 6 mmol/g, between 1.25 mmol/g and 6 mmol/g, between 1.5 mmol/g and 6 mmol/g, between 2.0 mmol/g and 6 mmol/g or between 2.5 mmol/g and 6 mmol/g, such as greater than 1.00, 1.5, 2.0, 2.5, 3.0, 3.5, 4, 4.5, 5.0, 5.5, 6.0, 7.0, 8.0, 9.0, 10.0, 11.0, 12.0 or greater than 13.0 mmol/g. The in vivo bile acid binding capacity may be measured in any animal, such as any mammal, such as humans or rats. The test methods detail a procedure for measuring the in vivo bile acid binding capacity in rats, which may be suitably modified as appropriate for measurement in humans.

One aspect of the invention is core-shell compositions comprising a polymeric core and shell. In some embodiments, the polymeric core comprises the crosslinked polyamine particles described herein. The shell material can be chemically anchored to the core material or physically coated. In the former case, the shell can be grown on the core component through chemical means, for example by: chemical grafting of shell polymer to the core using living polymerization from active sites anchored onto the core polymer; interfacial reaction, i.e., a chemical reaction located at the core particle surface, such as interfacial polycondensation; and using block copolymers as suspending agents during the core particle synthesis.

In some embodiments, the interfacial reaction and use of block polymers are the techniques used when chemical methods are used. In the interfacial reaction pathway, typically, the periphery of the core material is chemically modified by reacting small molecules or macromolecules on the core interface. For example, a crosslinked polyamine core is reacted with a polymer containing amine reactive groups such as epoxy, isocyanate, activated esters or halide groups to form a crosslinked shell around the core.

When the shell material is physically adsorbed on the core material, well known techniques of microencapsulation such as solvent coacervation, fluidized bed spray coater, or multiemulsion processes can be used. One method of microencapsulation is the fluidized bed spray coater in the Wurster configuration. In yet another embodiment, the shell material is only acting temporarily by delaying the swelling of the core while in the mouth and esophagus, and optionally disintegrates in the stomach or duodenum. The shell may be selected in order to hinder the transport of water into the core, by creating a layer of high hydrophobicity and very low liquid water permeability.

In some embodiments, shell materials are polymers carrying negative charges in the pH range typically found in the intestine. Examples include, but are not limited to, polymers that have pendant acid groups such as carboxylic, sulfonic, hydrosulfonic, sulfamic, phosphoric, hydrophosphoric, phosphonic, hydrophosphonic, phosphoramidic, phenolic, boronic and a combination thereof. The polymer can be protonated or unprotonated; in the latter case the acidic anion can be neutralized with pharmaceutically acceptable cations such as Na, K, Li, Ca, Mg, and NH₄.

The shell polymers can be either linear, branched, hyperbranched, segmented (i.e. backbone polymer arranged in sequence of contiguous blocks of which at least one contains pendant acidic groups), comb-shaped, star-shaped or crosslinked in a network, fully and semi-interpenetrated network (IPN). The shell polymers are either random or blocky in composition and either covalently or physically attached to the core material. Examples of such shell polymers include, but are not limited to acrylic acid homopolymers or copolymers, methacrylic acid homopolymers or copolymers, and copolymers of methacrylate and methacrylic acid. Examples of such polymers are copolymers of methyl methacrylate and methacrylic acid and copolymers of ethyl acrylate and methacrylic acid, sold under the tradename Eudragit (Rohm GmbH & Co. KG): examples of which include Eudragit L100-55 and Eudragit L100 (a methyl methacrylate-methacrylic acid (1:1) copolymer, Degussa/Rohm), Eudragit L30-D55, Eudragit S 100-55 and Eudragit FS 30D, Eudragit S 100 (a methyl methacrylate-methacrylic acid (2:1) copolymer), Eudragit LD-55 (an ethyl acrylate-methacrylic acid (1:1) copolymer), copolymers of acrylates and methacrylates with quaternary ammonium groups, sold under the tradenames Eudragit RL and Eudragit RS, and a neutral ester dispersion without any functional groups, sold under the tradename Eudragit NE30-D.

Additional shell polymers include: poly(styrene sulfonate), polyacrylic acid(s); carboxymethyl cellulose, cellulose acetate phthalate, hydroxypropyl methylcellulose phthalate as sold under the tradename HP-50 and HP-55 (Shin-Etsu Chemical Co., Ltd.), cellulose acetate trimellitate, cellulose acetate, cellulose acetate butyrate, cellulose acetate propionate, ethyl cellulose, cellulose derivatives, such as hydroxypropylmethylcellulose, methylcelluose, hydroxylethylcellulose, hydroxyethylmethylcellulose, hydroxylethylethylcelluose and hydroxypropylethylcellulose and cellulose derivatives such as cellulose ethers useful in film coating formulations, polyvinyl acetate phthalate, carrageenan, alginate, or poly(methacrylic acid) esters, acrylic/maleic acid copolymers, styrene/maleic acid polymers, itaconic acid/acrylic copolymers, and fumaric/acrylic acid copolymers, polyvinyl acetal diethylaminoacetate, as sold under the tradename AEA (Sankyo Co., Ltd.), methylvinylether/maleic acid copolymers and shellac.

In some embodiments the shell polymers are selected amongst pharmaceutically acceptable polymers such as Eudragit L100-55 and Eudragit L100 (a methylmethacrylate-methacrylic acid (1:1) copolymer, Degussa/Rohm), Carbopol 934 (polyacrylic acid, Noveon), C-A-P NF (cellulose acetate phthalate—Eastman), Eastacryl (methacrylic acid esters—Eastman), Carrageenan and Alginate (FMC Biopolymer), Anycoat-P (Samsung Fine Chemicals—HPMC Phthalate), or Aqualon (carboxymethyl cellulose—Hercules), methylvinylether/maleic acid copolymers (Gantrez), and styrene/maleic acid (SMA).

The shell can be coated by a variety of methods. In one embodiment, the shell materials are added in the drug formulation step as an active excipient; for example, the shell material can be included in a solid formulation as a powder, which is physically blended with the crosslinked polyamine and other excipients, optionally granulated, and compressed to form a tablet. Thus, in some embodiments, the shell material need not cover the core material in the drug product. For example, the acidic shell polymer may be added together with the core formulated in the shape of a tablet, capsule, gel, liquid, etc, wafer, extrudates and the shell polymer can then dissolve and distribute itself uniformly as a shell coating around the core while the drug product equilibrates in the mouth, esophagus or ultimately in the site of action, i.e. the GI tract.

In some embodiments, the shell is a thin layer of shell polymer. The layer can be a molecular layer of polyanion on the core material surface. The weight to core ratio can be between about 0.0001% to about 30%, preferably comprised between about 0.01% to about 5%, such as between about 0.1% to about 5%.

The shell polymers have a minimum molecular weight such that they do not freely permeate within the core pore volume nor elute from the core surface. In some embodiments, the molecular weight (Mw) of the shell acidic polymer is above about 1000 g/mole, such as above about 5000 g/mole, and or even above about 20,000 g/mole

The anionic charge density of the shell material (as prevailing in the milieu of use) may be between 0.5 mEq/g to 22 mEq/g, such as 2 mEq/g to 15 mEq/g. If a coating process is used to form the shell on the crosslinked polyamine particles as part of the manufacture of the dosage form, then procedures known from those skilled-in-the-art in the pharmaceutical industry are applicable. In one embodiment, the shell is formed in a fluidized bed coater (Wurster coater). In an alternate embodiment, the shell is formed through controlled precipitation or coascervation, wherein the crosslinked amine polymer particles are suspended in a polymer solution, and the solvent properties are changed in such a way as to induce the polymer to precipitate onto or coat the crosslinked amine polymer particles.

Suitable coating processes include the procedures typically used in the pharmaceutical industry. Typically, selection of the coating method is dictated by a number of parameters, that include, but are not limited to the form of the shell material (bulk, solution, emulsion, suspension, melt) as well as the shape and nature of the core material (spherical beads, irregular shaped, etc.), and the amount of shell deposited. In addition, the cores may be coated with one or more shells and may comprise multiple or alternating layers of shells.

The term “phosphate imbalance disorder” as used herein refers to conditions in which the level of phosphorus present in the body is abnormal. One example of a phosphate imbalance disorder includes hyperphosphatemia. The term “hyperphosphatemia” as used herein refers to a condition in which the element phosphorus is present in the body at an elevated level. Typically, a patient is often diagnosed with hyperphosphatemia if the blood phosphate level is, for example, above about 4.0 or 4.5 milligrams per deciliter of blood, for example above about 5.0 mg/dl, such as above about 5.5 mg/dl, for example above 6.0 mg/dl, and/or the patient has a severely impaired glomerular filtration rate such as, for example, less than about 20% of normal. The present invention may also be used to treat patients suffering from hyperphosphatemia in End Stage Renal Disease and who are also receiving dialysis treatment (e.g., hemodialysis or peritoneal dialysis). Also, the present invention can be used to treat Chronic Kidney Disease (CKD), to treat patients with CKD who are on dialysis and dialysis patients, including prophylactic treatment of any of the above.

Other diseases that can be treated with the methods, polymers, crosslinked polyamine particles, compositions and kits of the present invention include hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues including prophylactic treatment of any of the above.

The crosslinked polyamine particles and compositions described herein can be used as an adjunct to other therapies e.g. those employing dietary control of phosphorus intake, dialysis, inorganic metal salts and/or other polymer resins.

The compositions of the present invention are also useful in removing chloride, bicarbonate, oxalate, and bile acids from the gastrointestinal tract. Crosslinked polyamine particles removing oxalate compounds or ions find use in the treatment of oxalate imbalance disorders, such as oxalosis or hyperoxaluria that increases the risk of kidney stone formation. Crosslinked polyamine particles removing chloride compounds or ions find use in treating acidosis, heartburn, acid reflux disease, sour stomach or gastritis, for example. In some embodiments, the compositions of the present invention are useful for removing fatty acids, bilirubin, and related compounds. Some embodiments may also bind and remove high molecular weight molecules like proteins, nucleic acids, vitamins or cell debris.

The present invention provides methods, pharmaceutical compositions, and kits for the treatment of animals. The term “animal” or “animal subject” or “patient” as used herein includes humans as well as other mammals (e.g., in veterinary treatments, such as in the treatment of dogs or cats, or livestock animals such as pigs, goats, cows, horses) and other livestock animals such as chickens and the like. One embodiment of the invention is a method of removing phosphorous-containing compounds such as organophosphates or phosphate from the gastrointestinal tract, such as the stomach, small intestine or large intestine of an animal by administering an effective amount of the crosslinked polyamine particles described herein.

The term “treating” and its grammatical equivalents as used herein include achieving a therapeutic benefit and/or a prophylactic benefit. By therapeutic benefit is meant eradication, amelioration, or prevention of the underlying disorder being treated. For example, in a hyperphosphatemia patient, therapeutic benefit includes eradication or amelioration of the underlying hyperphosphatemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of crosslinked polyamine particles, described herein, to a patient suffering from renal insufficiency and/or hyperphosphatemia provides therapeutic benefit not only when the patient's serum phosphate level is decreased, but also when an improvement is observed in the patient with respect to other disorders that accompany renal failure and/or hyperphosphatemia like ectopic calcification and renal osteodistrophy. For prophylactic benefit, for example, the crosslinked polyamine particles may be administered to a patient at risk of developing hyperphosphatemia or to a patient reporting one or more of the physiological symptoms of hyperphosphatemia, even though a diagnosis of hyperphosphatemia may not have been made.

The compositions may also be used to control serum phosphate in subjects with elevated phosphate levels, for example, by changing the serum level of phosphate towards a normal or near normal level, for example, towards a level that is within 10% of the normal level of a healthy patient.

Other embodiments of the invention are directed towards pharmaceutical compositions comprising at least one of the crosslinked polyamine particles or a pharmaceutically acceptable salt of the crosslinked polyamine particles, and one or more pharmaceutically acceptable excipients, diluents, or carriers and optionally additional therapeutic agents. The compositions may be lyophilized or dried under vacuum or oven before formulating.

The excipients or carriers are “acceptable” in the sense of being compatible with the other ingredients of the formulation and not deleterious to the recipient thereof. The formulations can conveniently be presented in unit dosage form and can be prepared by any suitable method. The methods typically include the step of bringing into association the agent with the excipients or carriers such as by uniformly and intimately bringing into association the crosslinked amine polymer with the excipients or carriers and then, if necessary, dividing the product into unit dosages thereof.

The pharmaceutical compositions of the present invention include compositions wherein the crosslinked polyamine particles are present in an effective amount, i.e., in an amount effective to achieve therapeutic and/or prophylactic benefit. The actual amount effective for a particular application will depend on the patient (e.g. age, weight, etc.) the condition being treated; and the route of administration.

In some embodiments, crosslinked polyamine particles and compositions of the invention may reduce urinary phosphorous of a patient in need thereof by 5-100% of the elevation above normal urinary phosphorous levels, such as 10-75%, 25-65%, or 45-60%. Some embodiments may reduce urinary phosphorous by greater than 10%, greater than 20%, greater than 30%, greater than 40%, greater than 45%, greater than 50% or greater than 60% of the elevation above normal urinary phosphorous levels.

In some embodiments, crosslinked polyamine particles and compositions of the invention may reduce blood phosphate of a patient in need thereof by 5-100% of the elevation above normal blood phosphate levels, such as 10-75%, 25-65%, or 45-60% of the elevation above normal blood phosphate levels. Some embodiments may reduce blood phosphate levels by greater than 10%, greater than 20%, greater than 30%, greater than 40%, greater than 45%, greater than 50% or greater than 60% of the elevation above normal blood phosphate levels.

The dosages of the crosslinked polyamine particles in animals will depend on the disease being, treated, the route of administration, and the physical characteristics of the animal being treated. Such dosage levels in some embodiments for either therapeutic and/or prophylactic uses may be from about 1 μm/day to about 30 μm/day, for example from about 2 gm/day to about 20 μm/day, from about 2 μm/day to about 10 μm/day, from about 3 μm/day to about 9 μm/day, from about 3 μm/day to about 8 μm/day, from about 3 μm/day to about 7 gm/day, from about 3 μm/day to about 6 μm/day, from about 3 μm/day to about 5 μm/day, from about 4 μm/day to about 7 μm/day or from about 4 μm/day to about 6 μm/day. The dose of the crosslinked amine polymers described herein can be less than about 50 μm/day, less than about 40 μm/day, less than about 30 μm/day, less than about 20 μm/day, and less than about 10 μm/day.

Typically, the crosslinked polyamine particles can be administered before or after a meal, or with a meal. As used herein, “before” or “after” a meal is typically within two hours, preferably within one hour, more preferably within thirty minutes, most preferably within ten minutes of commencing or finishing a meal, respectively.

Generally, it is preferred that the crosslinked polyamine particles are administered along with meals. In some embodiments, the crosslinked polyamine particles may be administered one time a day, two times a day, or three times a day. In some embodiments, the crosslinked polyamine particles are administered once a day with the largest meal.

Preferably, the crosslinked polyamine particles may be used for therapeutic and/or prophylactic benefits and can be administered alone or in the form of a pharmaceutical composition. The pharmaceutical compositions comprise the crosslinked polyamine particles, one or more pharmaceutically acceptable carriers, diluents or excipients, and optionally additional therapeutic agents. For example, the crosslinked polyamine particles of the present invention may be co-administered with other active pharmaceutical agents depending on the condition being treated. Examples of pharmaceutical agents that may be co-administered include, but are not limited to:

Other phosphate sequestrants including pharmaceutically acceptable lanthanum, calcium, aluminum, magnesium, iron and zinc compounds, such as acetates, carbonates, oxides, hydroxides, citrates, alginates, and ketoacids thereof.

Calcium compounds, including calcium carbonate, acetate (such as PhosLo® calcium acetate tablets), citrate, alginate, and ketoacids;

Aluminium-based phosphate sequestrants, such as Amphojel® aluminium hydroxide gel;

Lanthanide compounds such as lanthanum carbonate (Fosrenol®).

Other phosphate sequestrants suitable for use in the present invention include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Other phosphate sequestrants suitable for co-administration include various examples of pharmaceutically acceptable zinc compounds are described in PCT Application No. PCT/US2005/047582 filed Dec. 29, 2005, the entire teachings of which are incorporated herein by reference. Specific suitable examples of pharmaceutically acceptable zinc compounds include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants described above can be used in the invention in combination with pharmaceutically acceptable ferric or ferrous iron salts.

In other embodiments, the phosphate sequestrant used in combination crosslinked polyamine particles of the present invention is not a pharmaceutically acceptable magnesium compound. In yet other embodiments, the phosphate sequestrant used in combination with the pharmaceutically acceptable crosslinked polyamine particles is not a pharmaceutically acceptable zinc compound.

The invention also includes methods and pharmaceutical compositions directed to a combination therapy of the crosslinked polyamine particles in combination with a phosphate transport inhibitor or an alkaline phosphatase inhibitor. Alternatively, a mixture of the crosslinked polyamine particles is employed together with a phosphate transport inhibitor or an alkaline phosphatase inhibitor.

Suitable examples of phosphate transport inhibitors can be found in co-pending U.S. Application Publication Nos. 2004/0019113 and 2004/0019020 and WO 2004/085448, the entire teachings of each of which are incorporated herein by reference.

Examples of alkaline phosphatase (ALP) inhibitors may be found in, for example, U.S. Pat. No. 5,948,630, the entire teachings of which are incorporated herein by reference. Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl)imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

This co-administration can include simultaneous administration of the two agents in the same dosage form, simultaneous administration in separate dosage forms, and separate administration. For example, for the treatment of hyperphosphatemia, the crosslinked polyamine particles may be co-administered with calcium salts which are used to treat hypocalcemia resulting from hyperphosphatemia.

The pharmaceutical compositions of the invention can be formulated as tablets, chewable tablets, sachets, slurries, food formulations, troches, capsules, elixirs, suspensions, syrups, wafers, chewing gums or lozenges.

Preferably, the crosslinked polyamine particles or the pharmaceutical compositions comprising the crosslinked polyamine particles are administered orally. Illustrative of suitable methods, vehicles, excipients and carriers are those described, for example, in Remington's Pharmaceutical Sciences, 19^{th} ed., the contents of which is incorporated herein by reference.

Pharmaceutical compositions for use in accordance with the present invention may be formulated in conventional manner using one or more physiologically acceptable carriers comprising excipients and auxiliaries which facilitate processing of the active crosslinked polyamine particles into preparations which can be used pharmaceutically. Proper formulation is dependent upon the route of administration chosen. Suitable techniques for preparing pharmaceutical compositions are well known in the art.

In some aspects of the invention, the crosslinked polyamine particles provide mechanical and thermal properties that are usually performed by excipients, thus decreasing the amount of such excipients required for the formulation. In some embodiments the crosslinked polyamine particles constitute over about 30 wt. %, for example over about 40 wt. %, over about 50 wt. %, preferably over about 60 wt. %, over about 70 wt. %, more preferably over about 80 wt. %, over about 85 wt. %, over about 90 wt. %, over about 95 wt. % or over about 99 wt. % of the composition, such as from about 80 wt. % to about 99 wt. % or from about 80 wt. % to about 95 wt. % of the composition, the remainder comprising suitable excipient(s).

In some embodiments, the dosage form of the composition is a tablet or tablets. In some embodiments, the compressibility of the tablets is strongly dependent upon the degree of hydration (moisture content) of the crosslinked polyamine particles. Preferably, the crosslinked polyamine particles have a moisture content of about 5% by weight or greater, more preferably, the moisture content is from about 5% to about 9% by weight, and most preferably about 7% by weight. It is to be understood that in embodiments in which the crosslinked polyamine particles are hydrated, the water of hydration is considered to be a component of the crosslinked polyamine particles.

The tablet can further comprise one or more excipients, such as hardeners, glidants and lubricants, which are well known in the art. Suitable excipients include colloidal silicon dioxide, stearic acid, magnesium silicate, calcium silicate, sucrose, calcium stearate, glyceryl behenate, magnesium stearate, talc, zinc stearate and sodium stearylfumarate.

In some embodiments, the tablets may be prepared by a method comprising the steps of: (1) hydrating or drying the crosslinked polyamine particles to the desired moisture level; (2) blending the crosslinked polyamine particles with any excipients; and (3) compressing the blend using conventional tableting technology to form a tablet or a tablet core. In some embodiments, the tablet or tablet core may then be further processed, such as coated.

In some embodiments, the invention relates to a stable, swallowable coated tablet, such as a tablet comprising the crosslinked polyamine particles, as described above. In one embodiment, the coating composition comprises a cellulose derivative and a plasticizing agent. The cellulose derivative is, preferably, hydroxypropylmethylcellulose (HPMC). The cellulose derivative can be present as an aqueous solution. Suitable hydroxypropylmethylcellulose solutions include those containing HPMC low viscosity and/or HPMC high viscosity. Additional suitable cellulose derivatives include cellulose ethers useful in film coating formulations. The plasticizing agent can be, for example, an acetylated monoglyceride such as diacetylated monoglyceride. The coating composition can further include a pigment selected to provide a tablet coating of the desired color. For example, to produce a white coating; a white pigment can be selected, such as titanium dioxide.

In one embodiment, a coated tablet of the invention can be prepared by a method comprising the step of contacting a tablet core, as described above, with a coating solution comprising a solvent, at least one coating agent dissolved or suspended in the solvent and, optionally, one or more plasticizing agents. Preferably, the solvent is an aqueous solvent, such as water or an aqueous buffer, or a mixed aqueous/organic solvent. Preferred coating agents include cellulose derivatives, such as hydroxypropylmethylcellulose. Typically, the tablet core is contacted with the coating solution until the weight of the tablet core has increased by an amount ranging from about 4% to about 6%, indicating the deposition of a suitable coating on the tablet core to form a coated tablet.

Other pharmaceutical excipients useful in some compositions of the invention include a binder, such as microcrystalline cellulose, carbopol, providone and xanthan gum; a flavoring agent, such as mannitol, xylitol, maltodextrin, fructose, or sorbitol; a lubricant, such as vegetable based fatty acids; and, optionally, a disintegrant, such as croscarmellose sodium, gellan gum, low-substituted hydroxypropyl ether of cellulose, sodium starch glycolate. Such additives and other suitable ingredients are well-known in the art; see, e.g., Gennaro A R (Ed.), Remington's Pharmaceutical Sciences, 19^{th} Edition.

In one embodiment, the crosslinked polyamine particles are pre-formulated with a high Tg/high melting point low molecular weight excipient such as mannitol, sorbose, and sucrose in order to form a solid solution wherein the crosslinked polyamine particles and the excipient are intimately mixed. Methods of mixing such as extrusion, spray-drying, chill drying, lyophilization, or wet granulation are useful. Indication of the level of mixing is given by known physical methods such as differential scanning calorimetry or dynamic mechanical analysis.

In some embodiments the crosslinked polyamine particles of the invention may be provided as pharmaceutical compositions in the form of liquid formulations. In some embodiments the pharmaceutical composition contains crosslinked polyamine particles dispersed in a suitable liquid excipient. Suitable liquid excipients are known in the art; see, e.g., Remington's Pharmaceutical Sciences.

In some embodiments, the pharmaceutical compositions may be in the form of a powder formulation packaged as a sachet that may be mixed with water or other ingestible liquid and administered orally as a drink (solution or suspension). In order to ensure that such formulations provide acceptable properties to the patient such as mouth feel and taste, a pharmaceutically acceptable anionic stabilizer may be included in the formulation.

Examples of suitable anionic stabilizers include anionic polymers such as: an anionic polypeptide, an anionic polysaccharide, or a polymer of one or more anionic monomers such as polymers of mannuronic acid, guluronic acid, acrylic acid, methacrylic acid, glucuronic acid glutamic acid or a combination thereof, and pharmaceutically acceptable salts thereof. Other examples of anionic polymers include cellulose, such as carboxyalkyl cellulose or a pharmaceutically acceptable salt thereof. The anionic polymer may be a homopolymer or copolymer of two or more of the anionic monomers described above. Alternatively, the anionic copolymer may include one or more anionic monomers and one or more neutral comonomers such as olefinic anionic monomers such as vinyl alcohol, acrylamide, and vinyl formamide.

Examples of anionic polymers include alginates (e.g. sodium alginate, potassium alginate, calcium alginate, magnesium alginate, ammonium alginate, and esters of alginate), carboxymethyl cellulose, polylactic acid, polyglutamic acid, pectin, xanthan, carrageenan, furcellaran, gum Arabic, karaya gum, gum ghatti, gum carob, and gum tragacanth. Preferred anionic polymers are alginates and are preferably esterified alginates such as a C₂-C₅-diol ester of alginate or a C₃-C₅ triol ester of alginate. As used herein an “esterified alginate” means an alginic acid in which one or more of the carboxyl groups of the alginic acid are esterified. The remainder of the carboxylic acid groups in the alginate are optionally neutralized (partially or completely) as pharmaceutically acceptable salts. For example, propylene glycol alginate is an ester of alginic acid in which some of the carboxyl groups are esterified with propylene glycol, and the remainder of the carboxylic acid groups is optionally neutralized with pharmaceutically acceptable salts. More preferably, the anionic polymer is ethylene glycol alginate, propylene glycol alginate or glycerol alginate, with propylene glycol alginate even more preferred.

### EXAMPLES

The following examples are of syntheses of some embodiments of the crosslinked polyamines that may be suitable for use in some embodiments of the crosslinked polyamine particles described herein. These examples are by way of example only and are not intended to limit the invention in any way.

As used herein, the following terms have the meanings ascribed to them unless specified otherwise:

PAMAM—A second generation starburst dendrimer having a diaminobutane core and 16 terminal amino groups was obtained from Dendritic Nanotechnologies, Inc.

DCM—Dichloromethane, commercially available from Sigma-Aldrich;

DAB-4—1,4-bis[bis(3-aminopropyl)amino]butane, commercially available from Aldrich;

DMAP—N,N-dimethylaminopyridine, commercially available from Aldrich;

Triton® B—trimethylbenzylammonium hydroxide, commercially available from Aldrich.

Proton NMR spectra were recorded at 400 MHz on a Varian NMR spectrometer in deuterated chloroform with TMS as an internal standard, unless otherwise indicated. ¹³C NMR experiments were performed on the same instrument, operating at a frequency of 66 MHz.

LC/MS experiments were performed on a Waters Ion Trap LC/MS equipped with a reversed phase Zorbax C-8 column. Samples were eluted with gradient mixtures of acetonitrile:water:formic acid. Ionization was performed using an electrospray source, with the ionization potential set to 30 V.

HPLC measurements were conducted on Agilent instruments, equipped with a Zorbax C-8 column, and an evaporative light scattering detector.

### Example 1

### Synthesis of an Amide Polyol

To a three-necked flask equipped with a magnetic stir bar was added 9 g of dimethyl L-tartarate, 12.85 g of tris(hydroxymethyl)aminomethane and 28 ml of methanol and the resulting solution was stirred at 50° C. for 20 hours. The addition of heat was stopped, and the solution was self-cooled to 42° C. and then filtered and dried at 30° C. in a vacuum oven for 20 hours to give 11.3 g of a white solid.

### Example 2

### Synthesis of Compound II

550 mg of PAMAM was added to 1.1 ml of deionized water and stirred. 20.96 μl of epichlorohydrin was added. A gel formed after stirring overnight at room temperature. The gel was broken into small pieces and suspended in 1.5 L of deionized water, filtered and dried in a forced air oven at 60° C.

### Example 3

### Synthesis of Compound III

6 g of a 20% solution of PAMAM in methanol was concentrated on a rotary evaporator. 7 g of deionized water was added to the concentrated PAMAM solution and stirred. 153 μl of epichlorohydrin was added. A gel formed after stirring overnight at room temperature. The gel was broken into small pieces, suspended in 2 L of deionized water, stirred and filtered. The filtered material was resuspended in 2 L of deionized water, stirred and filtered. The filtered polymer having a wet weight of 55.9 g was dried in a forced air oven at 60° C. to yield 700 mg of the desired product having an in-process-swelling ratio of 78.86 ml/g.

### Example 4

### Synthesis of Compound IV

A 101 g sample of pentaerythritol was charged to a 2 L 3-necked round bottom flask under N₂, and was slurried in 500 mL of acrylonitrile and 500 mL of 1,4-dioxane. A 9 mL portion of 40% KOH solution, and 18 mL of water were added to the reaction mixture, and the mixture stirred at room temperature. The reaction was heated to 40° C., at which point the pentaerythritol began dissolving. A slow exotherm began, and the reaction was cooled with ice to keep the temperature under 60° C. The reaction was stirred at room temperature overnight, and was analyzed by HPLC the following morning. The reaction mixture was transferred to a large separatory funnel, and was diluted with 2 L of tert-butyl methyl ether. The organic phase was then washed twice with 50% brine, was dried over anhydrous sodium sulfate, was filtered, and was concentrated in vacuo to yield 250 g of a light yellow oil, that solidified upon standing. The material was suitably pure to use for subsequent steps without further purification. ¹H NMR (300 MHz, CDCl₃): δ (ppm) 2.5 (t, 8 H); 3.4 (s, 8H); 3.6 (t, 8H). ¹³C NMR (66 MHz, CDCl₃): δ (ppm) 19.019 (CH₂CN); 45.802 (quaternary C); 65.842 (O—CH₂—CH₂); 68.909 (C—CH₂—O); 118.532 (—CN). HPLC purity (ELSD): >95% AUC.

### Example 5A

### Synthesis of Compound V

A 6 g sample of Compound IV was placed in a Parr hydrogenation apparatus, and was suspended in 150 mL of 1:1 MeOH:H₂O. 12 g of wet Raney cobalt catalyst were charged to this mixture, and the reaction vessel sealed. The resulting mixture was hydrogenated under 700 psi H₂ at 70° C. for 18 h. The reaction vessel was cooled to room temperature, the resulting material was analyzed by LC/MS and filtered through a bed of celite. The filtrate was concentrated in vacuo to yield 5.8 g of the desired product as a pale yellow oil. ¹H NMR (300 MHz, D₂O): δ (ppm) 1.7 (m, 8H); 2.5 (t, 8H); 3.2 (s, 8H); 3.4 (t, 8 H). HPLC purity (ELSD): >98% AUC. LC/MS [M+H]⁺ m/z=365.5 (exact mass of compound=364.300).

### Example 5B

### Synthesis of Compound V

A 50 g sample of Compound IV was placed in a Parr hydrogenation apparatus. To this, 5 g of freshly dried Raney cobalt was added in 30 mL of toluene, under N₂. The hydrogenation apparatus was sealed, and evacuated. 20 psi of anhydrous ammonia was introduced, followed by 1200 psi of hydrogen. The reaction mixture was then heated to 109° C., and was stirred for 12 hr at which point the resulting material was cooled to room temperature and analyzed by LC/MS before being filtered over a small amount of celite (under N₂), with the celite being washed several times with DCM. The filtrate was concentrated in vacuo to give 52 g of the desired product as a yellow oil. ¹H NMR (300 MHz, D₂O): δ (ppm) 1.7 (m, 8H); 2.5 (t, 8H); 3.2 (s, 8H); 3.4 (t, 8H). HPLC purity (ELSD): >98% AUC. LC/MS [M+H]⁺ m/z=365.5 (exact mass of compound=364.300).

### Example 6

### Synthesis of Compound VI

A 52 g sample of Compound V was charged to a Parr hydrogenation apparatus, along with 112 mL of acrylonitrile. The reaction vessel was sealed, and was evacuated. The vessel was then pressurized with 50 psi of N₂, and was heated at 140° C. for 12 h. The reaction vessel was cooled to room temperature and analyzed via HPLC. The reaction mixture was concentrated in vacuo to give ˜200 g of crude material. 40 g of this material were purified over normal phase silica gel (0-100% ethyl acetate:hexane mobile phase) to give 28 g of the desired product. HPLC purity (ELSD): >95% AUC. LC/MS [M+H]⁺ m/z=789.6 (exact mass of compound=788.52).

### Example 7A

### Synthesis of Compound VII

A 3 g sample of Compound VI was charged to a Parr hydrogenation apparatus, and was dissolved in a mixture of 150 mL methanol and 50 mL water. 12 g of wet Raney cobalt were added to the reaction vessel. The vessel was sealed, and the reaction hydrogenated at 80° C. under 1500 psi H₂ for 4 days. The reaction was cooled to room temperature and the analyzed via HPLC and LC/MS, filtered over a bed of celite, with the resulting light blue filtrate concentrated under reduced pressure. The resulting oil was suspended in a 1:1 mixture of methanol and DCM, and was dried over anhydrous sodium sulfate and then treated by excess ammonia in methanol. The resulting material was filtered over celite, and the filtrate concentrated in vacuo to yield 3 g of the desired product as a clear oil. HPLC purity (ELSD): >99% AUC.

### Example 7B

### Synthesis of Compound VII

A 28 g sample of Compound VI was charged to a Parr hydrogenation apparatus, along with 10 g of azeotropically dried Raney cobalt in 40 mL of toluene, under N₂. The reaction vessel was sealed, and evacuated. 40 psi of anhydrous ammonia were introduced, followed by 1600 psi H₂. The reaction was hydrogenated at 120° C. for 3 days, at which point it was cooled to room temperature, the resulting material analyzed by HPLC and LC/MS and filtered over celite (under N₂), with the filter pad being washed with several portions of DCM. The filtrate was concentrated in vacuo to yield 26 g of the desired product as a yellow oil. HPLC Purity (ELSD): >95% AUC. LC/MS [M+H]⁺ m/z=822.3 (molecular weight of compound=821.28, exact mass=820.77).

### Example 8

### Reaction of Compound VII with Epichlorohydrin

To a round bottomed flask was added 6.1 g of Compound IV, 6.1 ml of water, and 420 μl of epichlorohydrin. The resulting solution was stirred at room temperature for 1.5 hours, before being heated to 60° C. for 14 hours. The resulting solids were suspended in 1 L of water, and stirred 1 hour. At this time, the suspension had a conductivity of 319 μS, and a pH of 10.5. The suspension pH was then adjusted to 7 with HCl. The resultant gel was then filtered to give 119 g of polymer (in process swelling ratio=20). The product was, dried at 65° C. in an oven with a constant nitrogen stream for 18 hours, which afforded 2.8 g of a sticky solid. The material was re-swelled in water, and the pH was adjusted to 2 [using HCl]. It was then filtered and dried again. After drying for 3 days, a hygroscopic solid was obtained.

### Example 9

### Synthesis of Compound VIII

To a round bottom flask was added 36.4 g of D-sorbitol, 200 ml of 1,4-dioxane and 106 ml of acrylonitrile. The resulting solution was cooled to 5° C. on ice, to which was dropwise added a solution of Triton® B (5 ml in 50 mL of dioxane) via addition funnel. The reaction mixture was stirred at room temperature for 18 hours, and was then concentrated under reduced pressure. The resulting residue was taken up in DCM and transferred to a separatory funnel. The organic layer was washed twice with 50% brine. The brine layers were combined and further extracted with DCM. The DCM fractions were combined, dried over anhydrous sodium sulfate, filtered, and then concentrated in vacuo. The resulting residue was purified by flash chromatography over silica gel (0->90% ethyl acetate in hexanes as a mobile phase) to afford the desired product (55 g) as a light yellow oil.

### Example 10

### Synthesis of Compound IX

2.5 g of a chilled solution (2° C.) of tris(3-aminopropyl)amine in 2.5 ml of anhydrous methanol was added to an ice water bath chilled solution (2° C.) of 8.7 ml of methyl acrylate in 10 ml of anhydrous methanol. The solution was allowed to slowly warm to room temperature and was stirred six days at room temperature. The solution was concentrated on a rotary evaporator (bath temperature at 40° C.) yielding a light-yellow colored viscous oil. 50 ml of anhydrous methanol was added to this material and the solution was concentrated on a rotary evaporator (bath temperature 40° C.). The addition of anhydrous methanol (40 ml) and concentration on a rotary evaporator was repeated two additional times. The resulting material was dried in vacuo yielding 9.15 g of viscous oil.

### Example 11

### Synthesis of Compound X

10 g of a chilled solution (2° C.) of tris(2-aminoethyl)amine in 10 ml of anhydrous methanol was slowly added to an ice water bath chilled solution (0° C.) of 45 ml of methyl acrylate in 40 ml of anhydrous methanol. The solution was allowed to slowly warm to room temperature and stirred for six days at room temperature. The solution was concentrated on a rotary evaporator (bath temperature 40° C.) to yield a light yellow viscous oil. 50 ml of anhydrous methanol was added to this material and the solution was concentrated on a rotary evaporator (bath temperature 40° C.). The addition of anhydrous methanol (40 ml) and concentration on a rotary evaporator was repeated two additional times. The resulting material was dried in vacuo.

### Example 12

### Synthesis of Compound XI

10 g of a chilled solution (2° C.) of DAB-4 in 10 ml of anhydrous methanol was slowly added to an ice water bath chilled solution (0° C.) of 28 ml of methyl acrylate in 28 ml of anhydrous methanol. The solution was allowed to slowly warm to room temperature and stirred for five days at room temperature. The solution was concentrated on a rotary evaporator (bath temperature 40° C.) to afford a light yellow colored viscous oil. 50 ml of anhydrous methanol was added to this material and the solution was concentrated on a rotary evaporator (bath temperature 40° C.). The addition of anhydrous methanol (40 ml) and concentration on a rotary evaporator was repeated two additional times. The resulting material was dried in vacuo to afford 30.11 g of the desired product.

### Example 13

### Synthesis of Compound XII

A mixture of 10 g of Compound IX and 28.92 g of tris(3-aminopropyl)amine was heated at 75° C. for four days under a nitrogen atmosphere. The mixture was cooled to room temperature, 50 ml of methanol was added to the reaction mixture, and the resulting solution was slowly added, with stirring to 2 L of diethylether. The solution was allowed to settle, the solvent (mostly diethyl ether) was decanted from the precipitate, and the precipitate was dried in a vacuum oven at 30° C. The dried material was re-dissolved in methylene chloride and concentrated on a rotary evaporator (bath temperature 45° C.) in vacuo. A stream of nitrogen was blown over the residue overnight to yield 22.72 g of the desired product.

### Example 14

### Reaction of Compound XII with Epichlorohydrin

A mixture of 19.40 g of Compound XII and 19.04 g of deionized water was heated at 60° C. until a solution formed. 905 ul of epichlorohydrin was added to a 19.04 g aliquot of this solution. Within ten minutes of stirring at room temperature, a gel formed and was cured overnight at room temperature. After curing, the gel was broken into small pieces, suspended in 1 L of deionized water and the pH of the suspension was adjusted to 7.6 using concentrated HCl. The suspension was filtered, and the collected material was re-suspended in 1 L of deionized water, stirred and filtered. The resulting material, having a wet weight of 142 g, was dried in a forced air oven at 60° C. to afford 8.1 g of desired product having an In-Process Swelling Ratio of 16 ml/g.

### Example 15

### Reaction of Compound XII with Epichlorohydrin

1.07 ml of epichlorohydrin was added to a solution of 7.5 g of Compound XII in 7.5 g of water. Within ten minutes of stirring at room temperature, a gel formed and was cured over night at room temperature. After curing, the gel was broken into small pieces, suspended in 1 L of deionized water and the pH of the suspension was adjusted to 8 using concentrated HCl. The suspension was filtered, and the collected material was re-suspended in 1 L of deionized water, stirred and filtered. The resulting material, having a wet weight of 48 g, was dried in a forced air oven at 60° C. to afford 5.9 g of desired product having an In-Process Swelling Ratio of 7.1 ml/g.

### Example 16

### Reaction of Compound XII with Epichlorohydrin

Epichlorohydrin was added to a solution of Compound XII in water and cured for different periods of time according to the amounts and times in Table I below. After curing, the gel was broken into small pieces, suspended in 200 ml of deionized water, stirred, pH adjusted to 7.0 using concentrated HCL, and filtered. The material was dried in a forced air oven at 60° C. The results are summarized in Table I below.

TABLE I
Reaction of Compound XII with Epichlorohydrin

| | Amount of | | Amount of | | | In-Process |
|---|---|---|---|---|---|---|
| | Compound | Amount of | Epichlorohydrin | Curing | Yield | Swelling |
| Example | IV (g) | DI water (g) | (ul) | Conditions | (g) | (ml/g) |
| VII-1 | 0.787 | 0.788 | 28 | 1 day at room | 0.1 | 33.5 |
| | | | | temperature, 3 | | |
| | | | | days at 60° C. | | |
| VII-2 | 0.734 | 0.749 | 42 | 30 minutes at | 0.5 | 41 |
| | | | | room temperature, | | |
| | | | | 1 day at 60° C. | | |
| VII-3 | 0.836 | 0.844 | 64 | 30 minutes at | 0.68 | 22 |
| | | | | room temperature, | | |
| | | | | 1 day at 60° C. | | |

### Example 17

### Synthesis of Compound XIII

A mixture of 10 g of Compound IX and 21.3 ml of tris(2-aminoethyl)amine was heated at 75° C. for four days under a nitrogen atmosphere. The mixture was cooled to room temperature, 30 ml of dichloromethane was added to the reaction mixture, and the resulting solution was slowly added, with stirring to 2 L of diethylether. The solution was allowed to settle for 30 minutes, the solvent (mostly diethyl ether) was decanted from the precipitate. The precipitate was dissolved in methylene chloride with a little methanol, and concentrated on a rotary evaporator (bath temperature 45° C.). A stream of nitrogen was blown over the residue overnight to yield 23.89 g of the desired product.

### Example 18

### Reaction of Compound XIII with Epichlorohydrin

Epichlorohydrin was added to a solution of Compound XIII in water and cured for different periods of time according to the amounts and times in Table II below. After curing, the gel was broken into small pieces, suspended in 200 ml of deionized water, stirred, pH adjusted to 7.0 using concentrated HCL, and filtered. The material was then dried in a forced air oven at 60° C. The results are summarized in Table II below.

TABLE II
Reaction of Compound XIII with Epichlorohydrin

| | Amount of | | Amount of | | | In-Process |
|---|---|---|---|---|---|---|
| | Compound | Amount of | Epichlorohydrin | Curing | Yield | Swelling |
| Example | VIII (g) | DI water (g) | (ul) | Conditions | (g) | (ml/g) |
| IX-1 | 0.75 | 0.75 | 63.2 | 1 day at room | 0.62 | 18 |
| | | | | temperature | | |
| IX-2 | 0.75 | 0.75 | 84.2 | 1 day at room | 0.70 | 9.9 |
| | | | | temperature | | |
| IX-3 | 0.75 | 0.75 | 105.3 | 1 day at room | 0.74 | 8 |
| | | | | temperature | | |

### Example 19

### Reaction of Compound XIII with Epichlorohydrin

1.07 ml of epichlorohydrin was added to a stirred solution of 9.5 g of Compound XIII in 9.5 g of deionized water. With 20 minutes of stirring at room temperature, a gel formed and was cured for three days at room temperature. After curing, the gel was broken into pieces, suspended in 1 L of deionized water and the pH of the suspension was adjusted to 8.4 using concentrated HCl. The suspension was filtered and the collected material was resuspended in 1 L of water, stirred and filtered. The resulting material, having a wet weight of 149 g, was dried in a forced air oven at 60° C. to yield 7.9 g of the desired product having an In-Process Swelling Ratio of 18 ml/g.

### Example 20

### Reaction of Compound XIII with Epichlorohydrin

1.605 ml of epichlorohydrin was added to a stirred solution of 9.5 g of Compound XIII in 9.5 g of deionized water. Within 23 minutes of stirring at room temperature, a gel formed and was cured for three days at room temperature. After curing, the gel was broken into small pieces, suspended in 1 L of deionized water, adjusted to pH 13 with the addition of 50% NaOH, and then further adjusted down to a pH of approximately 8-9 using concentrated HCl. The suspension was filtered, and the collected material was re-suspended in 1 L of deionized water, stirred and filtered. The resulting material, having a wet weight of 72.82 g, was dried in a forced air oven at 60° C. to afford 9.78 g of desired product having an In-Process Swelling Ratio of 6.44 ml/g.

### Example 21

### Synthesis of Compound XIV

A mixture of 9.06 g of Compound IX and 25.5 ml of tris(3-aminopropyl)amine was heated at 75° C. for 48 hours under a nitrogen atmosphere. The mixture was cooled to room temperature, 30 ml of dichloromethane was added to the mixture and the resulting solution was slowly added with stirring to 1 L of tert-butyl methyl ether. The solution was stirred for five minutes, allowed to settle at 0° C., and the solvent (mostly tert-butyl methyl ether) was decanted from the precipitate. The precipitate was mixed in methylene chloride, and concentrated on a rotary evaporator (bath temperature 40° C.) under vacuum. The concentrated material was further dried overnight under vacuum, and a stream of nitrogen was blown over the residue overnight to yield 25.57 g of the desired product. This material was subsequently dissolved in 25.57 g of deionized water to afford a 50% (w/w) stock solution.

### Example 22

### Reaction of Compound XIV with Epichlorohydrin

1.81 ml of epichlorohydrin was added to 19 g of the 50% stock solution from Example 21. Within five minutes of stirring at room temperature, a gel formed and was cured over two nights at room temperature. After curing, the gel was broken into small pieces, suspended in 1 L of deionized water and the pH of the suspension was adjusted to 8 using concentrated HCl. The suspension was filtered, and the collected material was re-suspended in 1 L of deionized water, stirred and filtered. The resulting material, having a wet weight of 38.85 g, was dried in a forced air oven at 60° C. to afford 10.14 g of desired product having an In-Process Swelling Ratio of 2.83 ml/g.

### Example 23

### Reaction of Compound XIV with Epichlorohydrin

905 ul of epichlorohydrin was added to 19 g of the 50% stock solution from Example 21. Within 15 minutes of stirring at room temperature, a gel formed and was cured for ten days at room temperature. After curing, the gel was broken into small pieces, suspended in 1 L of deionized water and the pH of the suspension was adjusted to 8 using concentrated HCl. The suspension was filtered, and the collected material was re-suspended in 1 L of deionized water, stirred and filtered. The resulting material, having a wet weight of 58.55 g, was dried in a forced air oven at 60° C. to afford 8.65 g of desired product having having an In-Process Swelling Ratio of 5.77 ml/g.

### Example 24

### Synthesis of Compound XV

A mixture of 9.03 g of Compound IX and 19.2 ml of tris(2-aminoethyl)amine was heated at 75° C. for 48 hours under a nitrogen atmosphere. The mixture was cooled to room temperature, 30 ml of dichloromethane was added to the mixture and the resulting solution was slowly added with stirring to 2 L of tert-butyl methyl ether. The solution was stirred for five minutes, allowed to settle at 0° C., and the solvent (mostly tert-butyl methyl ether) was decanted from the precipitate. The precipitate was mixed with methylene chloride, and concentrated on a rotary evaporator (bath temperature 40° C.) under vacuum. The concentrated material was further dried overnight in vacuo, and a stream of nitrogen was blown over the residue overnight to yield 23.12 g of the desired product. This material was subsequently dissolved in 23.12 g of deionized water to afford a 50% (w/w) stock solution.

### Example 25

### Reaction of Compound XV with Epichlorohydrin

2.14 ml of epichlorohydrin was added to 19 g of the 50% stock solution from Example 24. Within ten minutes of stirring at room temperature, a gel formed and was cured for two days at room temperature. After curing, the gel was broken into small pieces, suspended in 1 L of deionized water and stirred at room temperature. The suspension was filtered, and the collected material was re-suspended in 1 L of deionized water, stirred and filtered. The resulting material, having a wet weight of 50.24 g, was dried in a forced air oven at 60° C. to afford 9.86 g of desired product having an In-Process Swelling Ratio of 4.10 ml/g.

### Example 26

### Reaction of Compound XV with Epichlorohydrin

1070 ul of epichlorohydrin was added to 19 g of the 50% stock solution from Example 24. Within 27 minutes of stirring at room temperature, a gel formed and was cured for two days at room temperature. After curing, the gel was broken into small pieces, suspended in 1 L of deionized water and the suspension was adjusted to pH 8 using concentrated HCl. The suspension was filtered, and the collected material was re-suspended in 1 L of deionized water, stirred and filtered. The resulting material, having a wet weight of 111.22 g, was dried in a forced air oven at 60° C. to afford 7.32 g of desired product having an In-Process Swelling Ratio of 14.19 ml/g.

### Example 27

### Synthesis of Compound XVI

A mixture of 9.04 g of Compound X and 20.4 ml of tris(2-aminoethyl)amine was heated at 75° C. for 48 hours under a nitrogen atmosphere. The mixture was cooled to room temperature, 30 ml of dichloromethane was added to the mixture and the resulting solution was slowly added with stirring to 2 L of tert-butyl methyl ether. The solution was stirred for five minutes, allowed to settle, the solvent (mostly tert-butyl methyl ether) was decanted from the precipitate and the precipitate was dried in vacuo overnight. The dried material was re-dissolved in methylene chloride and methanol and concentrated on a rotary evaporator (bath temperature 40° C.) under vacuum. The concentrated material was further dried overnight in vacuo, and a stream of nitrogen was blown over the residue overnight to yield 25.62 g of the desired product. This material was subsequently dissolved in 25.62 g of deionized water to afford a 50% (w/w) stock solution.

### Example 28

### Reaction of Compound XVI with Epichlorohydrin

1.5 ml of epichlorohydrin was added to 26 g of the 50% stock solution from Example 27. Within one hour and 40 minutes of stirring at room temperature, a gel formed and was cured for four days at room temperature. After curing, the gel was broken into small pieces, suspended in 2 L of deionized water and stirred at room temperature. The suspension was filtered, and the collected material was re-suspended in 2 L of deionized water, stirred and filtered. The resulting material, having a wet weight of 138.3 g, was dried in a forced air oven at 60° C. to afford 9.50 g of desired product having an In-Process Swelling Ratio of 13.56 ml/g.

### Example 29

### Synthesis of Compound XVII

A mixture of 9.03 g of Compound X and 27 ml of tris(3-aminopropyl)amine was heated at 75° C. for 48 hours under a nitrogen atmosphere. The mixture was cooled to room temperature, 30 ml of dichloromethane was added to the mixture and the resulting solution was slowly added with stirring to 2 L of tert-butyl methyl ether. The solution was stirred for five minutes, allowed to settle, the solvent (mostly tert-butyl methyl ether) was decanted from the precipitate and the precipitate was dried in a vacuum oven overnight. The dried material was re-dissolved in methylene chloride and methanol and concentrated on a rotary evaporator (bath temperature 40° C.) in vacuo. The concentrated material was further dried overnight in vacuo, and a stream of nitrogen was blown over the residue overnight to yield 28.31 g of the desired product. This material was subsequently dissolved in 28.31 g of deionized water to afford a 50% (w/w) stock solution.

### Example 30

### Reaction of Compound XVII with Epichlorohydrin

1.2 ml of epichlorohydrin was added to 22 g of the 50% stock solution from Example 29. Within 12 minutes of stirring at room temperature, a gel formed and was cured for four days at room temperature. After curing, the gel was broken into small pieces, suspended in 2 L of deionized water and stirred at room temperature. The suspension was filtered, and the collected material was re-suspended in 2 L of deionized water, stirred and filtered. The resulting material, having a wet weight of 63.71 g, was dried in a forced air oven at 60° C. to afford 9.0 g of desired product having an In-Process Swelling Ratio of 6.08 ml/g.

### Example 31

### Synthesis of Compound XVIII

A mixture of 9.09 g of Compound X and 45 ml of DAB-4 was heated at 75° C. for four days under a nitrogen atmosphere. The mixture was cooled to room temperature, 30 ml of dichloromethane was added to the mixture and the resulting solution was slowly added with stirring to 2 L of tert-butyl methyl ether. The solution was stirred for five minutes, allowed to settle, the solvent (mostly tert-butyl methyl ether) was decanted from the precipitate and the precipitate was dried in a vacuum oven overnight. The dried material was re-dissolved in methylene chloride and methanol and concentrated on a rotary evaporator (bath temperature 40° C.) in vacuo. The concentrated material was further dried overnight in vacuo, and a stream of nitrogen was blown over the residue overnight to yield 49.40 g of the desired product. This material was subsequently dissolved in 49.40 g of deionized water to afford a 50% (w/w) stock solution.

### Example 32

### Reaction of Compound XVIII with Epichlorohydrin

1.5 ml of epichlorohydrin was added to 36.2 g of the 50% stock solution from Example 31. Within 11 minutes of stirring at room temperature, a gel formed and was cured for four days at room temperature. After curing, the gel was broken into small pieces, suspended in 2 L of deionized water and stirred at room temperature. The suspension was filtered, and the collected material was re-suspended in 2 L of deionized water, stirred and filtered. The resulting material, having a wet weight of 58.78 g, was dried in a forced air oven at 60° C. to afford 10.0 g of desired product having an In-Process Swelling Ratio of 4.88 ml/g.

### Example 33

### Synthesis of Compound XIX

A mixture of 9.05 g of Compound IX and 42 ml of DAB-4 was heated at 75° C. for four days under a nitrogen atmosphere. The mixture was cooled to room temperature, 30 ml of dichloromethane was added to the mixture and the resulting solution was slowly added with stirring to 2 L of tert-butyl methyl ether. The solution was stirred for five minutes, allowed to settle, the solvent (mostly tert-butyl methyl ether) was decanted from the precipitate and the precipitate was dried in a vacuum oven overnight. The dried material was re-dissolved in methylene chloride and methanol and concentrated on a rotary evaporator (bath temperature 40° C.) in vacuo. The concentrated material was further dried overnight in vacuo, and a stream of nitrogen was blown over the residue overnight to yield 29.58 g of the desired product. This material was subsequently dissolved in 29.58 g of deionized water to afford a 50% (w/w) stock solution.

### Example 34

### Reaction of Compound XIX with Epichlorohydrin

1.3 ml of epichlorohydrin was added to 39.82 g of the 50% stock solution from Example 33. Within 19 minutes of stirring at room temperature, a gel formed and was cured for four days at room temperature. After curing, the gel was broken into small pieces, suspended in 2 L of deionized water and stirred at room temperature. The suspension was filtered, and the collected material was re-suspended in 2 L of deionized water, stirred and filtered. The resulting material, having a wet weight of 71.4 g, was dried in a forced air oven at 60° C. to afford 10.5 g of desired product having an In-Process Swelling Ratio of 5.8 ml/g.

### Example 35

### Synthesis of Compound XX

A mixture of Compound XI (29.54 g) and tris(2-aminoethyl)amine (60.0 mL) was heated at 75° C. for four days under a nitrogen atmosphere. The resulting solution was diluted with deionized water and dialyzed against deionized water (membrane MWCO 3,500). The dialyzed solution was concentrated and lyophilized to afford 34.16 g.

### Example 36

### Reaction of Compound XX with Epichlorohydrin

2.0 ml of epichlorohydrin was added to 17.25 g of a 50% (w/w) aqueous solution of Compound XX. Within 14 minutes of stirring at room temperature, a gel formed and was cured overnight at room temperature and for 8 hours at 60° C. After curing and cooling to room temperature, the gel was broken into small pieces, and suspended in 2 L of deionized water. The suspension was filtered, and the collected material was re-suspended in 2 L of deionized water, stirred and the pH was adjusted to 11 with 50% aqueous NaOH. This suspension was filtered, washed and filtered two more times with 2 L of deionized water each time. The resulting material having a wet weight of 74.5 g was dried in a forced air oven at 60° C. to afford 16.87 g of desired product having an In-Process Swelling of 3.42 ml/g.

### Example 37

### Synthesis of Compound XXI

A mixture of 30 g of Compound IX and 64 ml of tris(2-aminoethyl)amine was heated at 75° C. for 48 hours. The mixture was cooled to room temperature, diluted with deionized water to 25% (w/w) and dialyzed (MWCO 3500). Lyophilization of the dialyzed product afforded 32.47 g of the desired product.

### Example 38

### Reaction of Compound XXI with Epichlorohydrin

3.1 ml of epichlorohydrin was added to 55.16 g of a 50% (w/w) aqueous solution of Compound XXI. Within 17 minutes of stirring at room temperature, a gel formed and was cured overnight at room temperature. After curing, the gel was broken into small pieces, and suspended in 2 L of deionized water. The suspension was filtered, and the collected material was re-suspended in 2 L of deionized water, stirred and filtered. The resulting material having a wet weight of 182.69 g was dried in a forced air oven at 60° C. to afford 27.79 g of desired product having an In-Process Swelling of 5.57 ml/g.

### Example 39

### Synthesis of Compound XXII

A solution of 0.231 g of tris(3-chloropropyl)amine hydrochloride, 141 μl of tris(2-aminoethyl)amine, 1 ml of acetonitrile and 500 μl of deionized water was heated at 75° C. under a nitrogen atmosphere for 12 hours. A light colored gel was formed.

### Example 40

### Synthesis of Compound XXIII

A solution of 0.266 g of tris(3-chloropropyl)amine hydrochloride, 161 μl of tris(2-aminoethyl)amine, 1 ml of acetonitrile and 500 μl of deionized water was heated at 75° C. under a nitrogen atmosphere for 12 hours. A light colored gel was formed.

### Example 41

### Synthesis of Compound XXIV

A solution of 0.253 g of tris(2-chloroethyl)amine hydrochloride, 175 μl of dipropylenetriamine, 1 ml of acetonitrile and 500 μl of deionized water was heated at 75° C. under a nitrogen atmosphere for 12 hours. A light colored gel was formed.

### Example 42

### Synthesis of Compound XXV

A solution of 0.5 g of tris(3-chloropropyl)amine hydrochloride, 5 ml of deionized water and 1.0 g of a 50% aqueous solution of NaOH was extracted twice with 6 ml of hexane. The hexane extracts were combined and concentrated in vacuo on a rotary evaporator to yield 0.419 g of tris(3-chloropropyl)amine. The resulting 0.419 g of tris(3-chloropropyl)amine was placed into solution with 1.3 ml of tris(2-aminoethyl)amine, 850 μl of acetonitrile and 850 μl of deionized water and was heated under a nitrogen atmosphere at 75° C. for 12 hours. The viscosity of the solution increased. The solution was concentrated in vacuo and diluted with methanol. The resulting solution was washed twice with diethyl ether and dried under a stream of nitrogen, followed by drying under vacuum over P₂O₅ to yield the desired product having a MW of 6.84 kD and a polydispersity of 1.54.

### Example 43

### Synthesis of Compound XXVI

A solution of 1.0 g of tris(3-chloropropyl)amine hydrochloride, 5 ml of deionized water and 1.0 g of a 50% aqueous solution of NaOH was extracted twice with 6 ml of hexane. The hexane extracts were combined and concentrated in vacuo on a rotary evaporator to afford 0.834 g of tris(3-chloropropyl)amine. The resulting 0.834 g of tris(3-chloropropyl)amine was placed into solution with 101 μl of tris(2-aminoethyl)amine, 450 μl of acetonitrile and 450 μl of deionized water and was heated under a nitrogen atmosphere at 75° C. for 12 hours. The solution formed a gel.

### Example 44

### Synthesis of Compound XXVII

A solution of 0.5 g of tris(2-chloroethyl)amine hydrochloride, 5 ml of deionized water and 1.0 g of a 50% aqueous solution of NaOH was extracted twice with 6 ml of hexane. The hexane extracts were combined and concentrated in vacuo on a rotary evaporator to yield 0.36 g of tris(2-chloroethyl)amine. The resulting 0.36 g of tris(2-chloroethyl)amine was placed into solution with 1.3 ml of dipropylenetriamine, 800 μl of acetonitrile and 800 μl of deionized water and was heated under a nitrogen atmosphere at 75° C. for 12 hours. The viscosity of the solution increased. The solution was concentrated in vacuo and diluted with methanol. The resulting solution was washed twice with diethyl ether and dried under a stream of nitrogen, followed by drying under vacuum over P₂O₅ to yield the desired product having a MW of 2.21 kD and a polydispersity of 1.74.

### Example 45

### Synthesis of Compound XXVIII

A solution of 0.6872 g of tris(2-chloroethyl)amine hydrochloride, 400 μl of dipropylenetriamine, 400 μl of acetonitrile and 400 μl of deionized water was heated at 75° C. under a nitrogen atmosphere for 12 hours. A gel was formed.

### Example 46

### Synthesis of Compound XIX

A solution of 2.0 g of tris(3-chloropropyl)amine hydrochloride, 20 ml of deionized water and 4.0 μg of a 50% aqueous solution of NaOH was extracted twice with 20 ml of hexane. The hexane extracts were combined and concentrated in vacuo on a rotary evaporator to yield 1.54 g of tris(3-chloropropyl)amine. The resulting 1.54 g of tris(3-chloropropyl)amine was placed into solution with 5.1 ml of tris(2-aminoethyl)amine, 3.4 ml of acetonitrile and 3.4 ml of deionized water and was heated under a nitrogen atmosphere at 75° C. for 72 hours. The viscosity of the solution increased. The solution was diluted with isopropanol and mixed with tert-butyl methyl ether. The resulting precipitate was collected and washed multiple times with isopropanol and t-butyl methyl ether mixtures. The residue was dried in vacuo. The isopropanol/tert-butyl methyl ether combined layers were concentrated and the residue and the concentrated layers were dissolved in deionized water: A 50% aqueous solution of NaOH was added until the solution pH was 10.6. The solution was dialyzed (MWCO 3500) against deionized water and lyophilized to afford 0.5 g of the desired product.

### Example 47

### Synthesis of Compound XXX

A solution of 0.2 g of tris(3-chloropropyl)amine hydrochloride, 550 μl of tris(2-aminoethyl)amine and 728 μl of deionized water was heated under a nitrogen atmosphere at 75° C. for 48 hours. The solution was diluted with isopropanol and concentrated HCl was added until the pH was between 2 and 3 as measured by pH paper. The solution was decanted from the precipitate and the precipitate was washed with isopropanol followed by tert-butyl methyl ether and dried in vacuo. The residue was dissolved in deionized water and a 50% solution of NaOH was added until the pH was 11. The solution was dialyzed (MWCO 3500) against deionized water and lyophilized to afford 25 mg of the desired product.

### Example 48

### Synthesis of Compound XXXI

A solution of 2.05 g of tris(2-chloroethyl)amine hydrochloride, 6.07 g of tris(2-aminoethyl)amine and 8 ml of deionized water was heated under a nitrogen atmosphere at 75° C. for 72 hours, yielding the desired product. The resulting product had a MW of 1.7 kD and a poyldispersity of 1.46.

### Example 49

### Synthesis of Compound XXXII

A solution of 2.03 g of tris(2-chloroethyl)amine hydrochloride, 3.64 g of tris(2-aminoethyl)amine and 5.6 ml of deionized water was heated under a nitrogen atmosphere at 75° C. for 72 hours, yielding the desired product. The resulting product had a MW of 2.48 kD and a poyldispersity of 1.93.

### Example 50

### Synthesis of Compound XXXIII

A solution of 2.04 g of tris(2-chloroethyl)amine hydrochloride, 7.8 g of tris(3-aminopropyl)amine and 9.8 ml of deionized water was heated under a nitrogen atmosphere at 75° C. for 72 hours, yielding the desired product. The resulting product had a MW of 2.4 kD and a poyldispersity of 1.45.

### Example 51

### Synthesis of Compound XXXIV

A solution of 2.05 g of tris(2-chloroethyl)amine hydrochloride, 4.69 g of tris(3-aminopropyl)amine and 9.8 ml of deionized water was heated under a nitrogen atmosphere at 75° C. for 72 hours, yielding the desired product. The resulting product had a MW of 2.4 kD and a poyldispersity of 1.45.

### Example 52

### Synthesis of Compound XXXV

A solution of 5.0 g of tris(3-chloropropyl)amine hydrochlroide, 50 ml of deionized water and 10.0 g of a 50% aqueous solution of NaOH was extracted twice with 60 ml of hexane each. The hexane extracts were combined and concentrated in vacuo on a rotary evaporator to yield 3.84 g of tris(3-chloropropyl)amine. The resulting 3.84 g of tris(3-chloropropyl)amine was placed into solution with 11.4 ml of tris(2-aminoethyl)amine, 5 ml of acetonitrile and 5 ml of deionized water and was heated under a nitrogen atmosphere at 75° C. for 6 days. The solution was diluted with methanol and the resulting precipitate was collected and concentrated in vacuo using a rotary evaporator. The material was dissolved into methanol and precipitated into tert-butyl methyl ether. The solvent layer was decanted and the residue was dried under a stream of nitrogen to afford 17.62 g of the desired product.

### Example 53

### Reaction of Compound XXXV with Epichlorohydrin

A solution of 2.0 g of Compound XXXV and 350 μl of epichlorohydrin in 2.0 g of deionized water was stirred overnight at room temperature resulting in a gel. The material was heated at 60° C. overnight and cooled to room temperature. The gel was broken into small pieces, suspended in 500 ml of deionized water, stirred and filtered. The wet material having a wet weight of 62.45 g was dried in a forced air oven at 60° C. to yield 0.75 g of product having an in-process-swelling ratio of 82.3 ml/g.

### Example 54

### Synthesis of Compound XXXVI

A solution of 5.0 g of tris(2-chloroethyl)amine hydrochloride, 50 ml of deionized water and 10.0 g of a 50% aqueous solution of NaOH was extracted twice with 60 ml of hexane each. The hexane extracts were combined and concentrated in vacuo on a rotary evaporator to yield 3.84 g of tris(2-chloroethyl)amine. The resulting 3.68 g of tris(2-chloroethyl)amine was placed into solution with 18.2 ml of tris(3-aminopropyl)amine, 6.5 ml of acetonitrile and 6.5 ml of deionized water and was heated under a nitrogen atmosphere at 75° C. for 6 days. The solution was diluted with methanol and the resulting precipitate was collected by filtration and concentrated in vacuo using a rotary evaporator. The material was dissolved into methanol and precipitated into t-butyl methyl ether. The solvent layer was decanted and the residue was dried under a stream of nitrogen to afford 24.42 g of the desired product.

### Example 55

### Reaction of Compound XXXVI with Epichlorohydrin

A solution of 2.0 g of Compound XXXVI and 350 μl of epichlorohydrin in 2.0 g deionized water was stirred overnight at room temperature resulting in a gel. The material was heated at 60° C. overnight and cooled to room temperature. The gel was broken into small pieces, suspended in 500 ml of deionized water, stirred and filtered. The wet material having a wet weight of 46.92 g was dried in a forced air oven at 60° C. to yield 1.10 g of product having an in-process-swelling ratio of 45.9 ml/g.

### Example 56

### Synthesis of Compound XXXVII

Four solutions of 2.0 g of tris(2-chloroethyl)amine hydrochloride, 5.0 ml of tris(3-aminopropyl)amine and 6.7 ml of deionized water were placed into separate reaction vials, and heated to 75° C. under a nitrogen atmosphere for 4 days. Isopropanol was added to each solution and the solutions were separately precipitated into t-butyl methyl ether. Each of the solvent layers was decanted off and the residues were taken up in methanol. The methanol solutions were combined, filtered through filter paper and concentrated in vacuo using a rotary evaporator. The residue was dried under a stream of nitrogen to yield 28.4 g of desired product.

### Example 57

### Reaction of Compound XXXVII with Epichlorohydrin

A solution of 2.0 g of Compound XXXVII and 300 μl of epichlorohydrin in 2.0 g deionized water was stirred overnight at room temperature resulting in a gel. The material was heated at 60° C. overnight and cooled to room temperature. The gel was broken into small pieces, suspended in 500 ml of deionized water, stirred and filtered. The wet material having a wet weight of 29.49 g was dried in a forced air oven at 60° C. to yield 1.3 g of product having an in-process-swelling ratio of 21.7 ml/g.

### Example 58

### Synthesis of Compound XXXVIII

A solution of 1.0 g of tris(2-chloroethyl)amine hydrochloride, 1.7 ml of tris(3-aminopropyl)amine and 1.2 ml of deionized water was heated under a nitrogen atmosphere at 75° C. for 72 hours. Methanol was added to the solution and the solution was concentrated in vacuo using a rotary evaporator. The resulting material was dissolved into methanol and precipitated into diethyl ether, the solvent layer was decanted and the residue was dried in a vacuum oven at room temperature. Deionized water was added to the dried residue to make a 50% aqueous solution, 390 μl of epichlorohydrin was added and the solution was stirred overnight at room temperature and then heated to 60° C. overnight. A gel formed within 20 minutes. After cooling to room temperature, the gel was broken into small pieces, suspended in deionized water, stirred and filtered. The wet material having a wet weight of 12.92 g was dried in a forced air oven at 60° C. to yield 2.07 g of product having an in-process-swelling ratio of 5.2 ml/g.

### Example 59

### Synthesis of Compound XXXIX

A solution of 1.0 g of tris(2-chloroethyl)amine hydrochloride, 2.1 ml of tris(3-aminopropyl)amine and 1.4 ml of deionized water was heated under a nitrogen atmosphere at 75° C. for 72 hours. Methanol was added to the solution and the solution was concentrated in vacuo using a rotary evaporator. The resulting material was dissolved into methanol and precipitated into diethyl ether, the solvent layer was decanted and the residue was dried in a vacuum oven at room temperature. Deionized water was added to the dried residue to make a 50% aqueous solution followed by 450 μl of epichlorohydrin and the solution was stirred overnight at room temperature and then heated to 60° C. overnight. A gel formed within 20 minutes. After cooling to room temperature, the gel was broken into small pieces, suspended in deionized water, stirred and filtered. The wet material having a wet weight of 22.78 g was dried in a forced air oven at 60° C. to yield 2.25 g of product having an in-process-swelling ratio of 9.12 ml/g.

### Example 60

### Synthesis of Compound XL

A solution of 1.0 g of tris(2-chloroethyl)amine hydrochloride, 2.5 ml of tris(3-aminopropyl)amine and 1.6 ml of deionized water was heated under a nitrogen atmosphere at 75° C. for 72 hours. Methanol was added to the solution and the solution was concentrated in vacuo using a rotary evaporator. The resulting material was dissolved into methanol and precipitated into diethyl ether, the solvent layer was decanted and the residue was dried in a vacuum oven at room temperature. Deionized water was added to the dried residue to make a 50% aqueous solution followed by 450 μl of epichlorohydrin. A gel formed and was cured for 3 days at room temperature. The gel was broken into small pieces, suspended in 1 L of deionized water, stirred and filtered. The wet material having a wet weight of 43.32 g was dried in a forced air oven at 60° C. to yield 2.0 g of product having an in-process-swelling ratio of 20.66 ml/g.

### Example 61

### Synthesis of Compound XLI

A solution of 1.0 g of tris(2-chloroethyl)amine hydrochloride, 1.3 ml of tris(2-aminoethyl)amine and 1.1 ml of deionized water was heated under a nitrogen atmosphere at 75° C. for 72 hours. Methanol was added to the solution and the solution was concentrated in vacuo using a rotary evaporator. The resulting material was dissolved into methanol and precipitated into diethyl ether, the solvent layer was decanted and the residue was dried in a vacuum oven at room temperature. Deionized water was added to the dried residue to make a 50% aqueous solution followed by 300 μl of epichlorohydrin and the solution was stirred overnight at room temperature and then heated to 60° C. overnight. A gel formed within 20 minutes. After cooling to room temperature, the gel was broken into small pieces, suspended in 1 L of deionized water, stirred and filtered. The wet material having a wet weight of 59.8 g was dried in a forced air oven at 60° C. to yield 0.85 g of product having an in-process-swelling ratio of 71 ml/g.

### Example 62

### Synthesis of Compound XLII

A solution of 5.0 g of tris(2-chloroethyl)amine hydrochloride, 8.5 ml of tris(3-aminopropyl)amine and 6 ml of deionized water was heated under a nitrogen atmosphere at 75° C. for 4 days. Methanol was added to the solution and the solution was concentrated in vacuo using a rotary evaporator. The methanol addition and concentration was repeated. The resulting material was dissolved into a small amount of methanol and precipitated into 0.75 L of diethyl ether. The solution was allowed to settle for two hours, the solvent layer was decanted and the residue was dried in a vacuum oven at 30° C. 13.5 g of deionized water was added to the dried residue followed by 2.0 ml of epichlorohydrin and the solution was stirred overnight at room temperature and then heated to 60° C. overnight. A gel formed. After cooling to room temperature, the gel was broken into small pieces, suspended in 2 L of deionized water, stirred, filtered, resuspended in 2 L of deionized water, stirred and filtered. The wet material having a wet weight of 122.46 g was dried in a forced air oven at 60° C. to yield 11.0 g of product having an in-process-swelling ratio of 10.13 ml/g.

### Example 63

### Synthesis of Compound XLIII

A solution of 5.0 g of tris(2-chloroethyl)amine hydrochloride, 12.5 ml of tris(3-aminopropyl)amine and 8 ml of deionized water was heated under a nitrogen atmosphere at 75° C. for 4 days. Methanol was added to the solution and the solution was concentrated in vacuo using a rotary evaporator. The methanol addition and concentration was repeated. The resulting material was dissolved into a small amount of methanol and precipitated into 0.75 L of diethyl ether. The solution was allowed to settle for two hours, the solvent layer was decanted and the residue was dried in a vacuum oven at 30° C. 17.5 g of deionized water was added to the dried residue followed by 2.5 ml of epichlorohydrin and the solution was stirred overnight at room temperature and then heated to 60° C. overnight. A gel formed. After cooling to room temperature, the gel was broken into small pieces, suspended in 2 L of deionized water, stirred, filtered, resuspended in 2 L of deionized water, stirred and filtered. The wet material having a wet weight of 537.08 g was dried in a forced air oven at 60° C. to yield 9.62 g of product having an in-process-swelling ratio of 54.83 ml/g.

### Example 64

### Synthesis of Compound XLIV

A solution of 10.0 g of tris(2-chloroethyl)amine hydrochloride, 13.0 ml of tris(2-aminoethyl)amine and 11 ml of deionized water was heated under a nitrogen atmosphere at 75° C. for 4 days. Methanol was added to the solution and the solution was concentrated in vacuo using a rotary evaporator. The methanol addition and concentration was repeated. The resulting material was dissolved into a small amount of methanol and precipitated into 0.75 L of diethyl ether. The solution was allowed to settle for two hours, the solvent layer was decanted and the residue was dried in a vacuum oven at 30° C. 23.0 g of deionized water was added to the dried residue followed by 3.3 ml of epichlorohydrin and the solution was stirred overnight at room temperature and then heated to 60° C. overnight. A gel formed. After cooling to room temperature, the gel was broken into small pieces, suspended in 2 L of deionized water, stirred, filtered, resuspended in 2 L of deionized water, stirred and filtered. The wet material having a wet weight of 606.73 g was dried in a forced air oven at 60° C. to yield 15.17 g of product having an in-process-swelling ratio of 39 ml/g.

### Example 65

### Synthesis of Compound XLV

9.8 g of Compound XLII was suspended in 2 L of deionized water with stirring, pH adjusted to 11.3 using a 50% aqueous solution of NaOH and filtered. The collected material was resuspended in 2 L of deionized water with stirring and pH adjusted to 9.6 using a 50% aqueous solution of NaOH. The suspension was filtered and the collected material was dried in a forced air oven at 60° C. to yield 6.55 g of the desired product.

### Example 66

### Synthesis of Compound XLVI

9.8 g of Compound XLII was suspended in 2 L of deionized water with stirring, pH adjusted to 11.2 using a 50% aqueous solution of NaOH and filtered. The collected material was resuspended in 2 L of deionized water with stirring and pH adjusted to 9.5 using concentrated HCl. The suspension was filtered and the collected material was dried in a forced air oven at 60° C. to yield 7.03 g of the desired product.

### Example 67

### Synthesis of Compound XLVII

A solution of 5.82 g of tris(3-chloropropyl)amine, 8.2 ml of tris(3-aminopropyl)amine and 6 ml of deionized water was heated under a nitrogen atmosphere at 75° C. overnight to form a gel. After cooling to room temperature, the gel was broken into small pieces, suspended in 2 L of deionized water and stirred. The pH of the suspension was adjusted to 11 using a 50% aqueous solution of NaOH and filtered.

### Example 68

### Synthesis of Compound XLVIII

A solution of 12.6 g of tris(2-chloroethyl)amine hydrochloride, 22.5 ml of tris(3-aminopropyl)amine and 16 ml of deionized water was heated under a nitrogen atmosphere at 75° C. for 4 days. Methanol was added to the solution and the solution was concentrated in vacuo using a rotary evaporator. The resulting material was dissolved into a small amount of methanol and precipitated into diethyl ether. The solution was allowed to settle, the solvent layer was decanted and the residue was dried in a vacuum oven at 30° C. 30 g of deionized water and a small amount of methanol was added to the dried residue and the solution was concentrated on a rotary evaporator to 69.32 g. 5.2 ml of epichlorohydrin was added to the resulting solution and the solution was stirred overnight at room temperature and then heated to 60° C. overnight. A gel formed. After cooling to room temperature, the gel was broken into small pieces, suspended in 2 L of deionized water, stirred, filtered, resuspended in 2 L of deionized water, stirred and filtered. The filtered material was suspended in 2 L of deionized water and adjusted to pH 11 with a 50% aqueous solution of NaOH and filtered. The resulting filtered material was suspended in 2 L of deionized water and filtered. The wet material having a wet weight of 182.1 g was dried in a forced air oven at 60° C. to yield 22.3 g of the desired product having an in-process-swelling ratio of 7.17 ml/g.

### Example 69

### Synthesis of Compound XLIX

A solution of 20.08 g of tris(2-chloroethyl)amine hydrochloride, 27 ml of tris(2-aminoethyl)amine and 22 ml of deionized water was heated under a nitrogen atmosphere at 75° C. for 4 days. Methanol was added to the solution and the solution was concentrated in vacuo using a rotary evaporator. The resulting material was dissolved into a small amount of methanol and precipitated into diethyl ether. The solution was allowed to settle, the solvent layer was decanted and the residue was dried in a vacuum oven at 30° C. 47 g of deionized water and a small amount of methanol was added to the dried residue and the solution was concentrated on a rotary evaporator to 78.56 g. 7.8 ml of epichlorohydrin was added to the resulting solution and the solution was heated to 60° C. for 3 hours at which point an additional 500 μl of epichlorohydrin was added and heating to 60° C. continued overnight. A gel formed. After cooling to room temperature, the gel was broken into small pieces, suspended in 2 L of deionized water, stirred, filtered, resuspended in 2 L of deionized water, stirred and filtered. The filtered material was suspended in 2 L of deionized water and adjusted to pH 11.2 with a 50% aqueous solution of NaOH and filtered. The resulting filtered material was suspended in 2 L of deionized water and stirred. The suspension was pH adjusted to 9.57 with concentrated HCl and filtered. The wet material having a wet weight of 499.6 g was dried in a forced air oven at 60° C. to yield 27.39 g of product having an in-process-swelling ratio of 17.24 ml/g. The dried material was suspended in 2 L of deionized water, stirred and filtered. The filtered material was suspended in 2 L of deionized water, stirred, pH adjusted to pH 12.6 using a 50% aqueous NaOH solution and filtered. The filtered material was suspended in 2 L of deionized water, stirred, filtered, resuspended in 2 L of deionized water, stirred and filtered. The filtered material was suspended in 2 L of deionized water, adjusted to pH 9.6 with concentrated HCl and filtered. The wet material having a wet weight of 164.7 was dried in a forced air oven at 60° C. to afford 24.42 g of the desired product having an in-process-swelling ratio of 5.74 ml/g.

### Example 70

### Synthesis of Compound L

A solution of 10.07 g of tris(3-chloropropyl)amine hydrochloride, 22.5 ml of tris(3-aminopropyl)amine and 13.3 ml of deionized water was heated under a nitrogen atmosphere at 75° C. for 3 days. Methanol was added to the solution and the solution was concentrated in vacuo using a rotary evaporator. The resulting material was dissolved into a small amount of methanol and precipitated into diethyl ether. The solution was allowed to settle, the solvent layer was decanted and the residue was dried in a vacuum oven at 30° C. 32.5 g of deionized water and a small amount of methanol was added to the dried residue and the solution was concentrated on a rotary evaporator to 60.61 g. 4.8 ml of epichlorohydrin was added to the resulting solution and the solution gelled within 25 minutes at room temperature. The gel was heated at 60° C. overnight. After cooling to room temperature, the gel was broken into small pieces, suspended in 2 L of deionized water, stirred, filtered, resuspended in 2 L of deionized water, stirred and filtered. The filtered material was suspended in 2 L of deionized water and adjusted to pH 9.4 with a 50% aqueous solution of NaOH and filtered. The resulting filtered material was dried in a forced air oven at 60° C. to afford 33.86 g. The dried material was suspended in 2 L of deionized water, stirred, filtered, resuspended in 2 L of deionized water, pH adjusted to 12.4 using a 50% aqueous solution of NaOH and filtered. The resulting material was washed twice by suspending it in 2 L of deionized water, stirring and filtering the suspension. The material resulting from the second filtration was resuspended in 2 L of deionized water, pH adjusted to 10 using a 50% aqueous solution of NaOH and filtered. The wet material having wet weight of 114.8 g was dried in a forced air oven at 60° C. to afford 22.26 g of the desired product having an in-process-swelling ratio of 4.2 ml/g.

### Example 71

### Synthesis of Compound LI

A solution of 10.08 g of tris(2-chloroethyl)amine hydrochloride, 24 ml of dipropylenetriamine and 14 ml of deionized water was heated under a nitrogen atmosphere at 90° C. for 4 days. Methanol was added to the solution and the solution was concentrated in vacuo using a rotary evaporator. The resulting material was dissolved into a small amount of methanol and precipitated into 1 L of diethyl ether. The solution was allowed to settle, the solvent layer was decanted. The residue was dissolved in a small amount of ethanol and precipitated into 1 L of diethyl ether. The solution was allowed to settle, the solvent layer was decanted and the residue was dried in a vacuum oven at 30° C. 34 g of deionized water was added to the dried residue and 6.0 ml of epichlorohydrin was added to the resulting solution. After stirring overnight at room temperature 600 μl of epichlorohydrin was added and the solution was heated overnight at 60° C. An additional 600 μl of epichlorohydrin was added and the solution was kept at room temperature for 8 hours, followed by heating at 60° C. overnight. A gel formed and was cured at 60° C. for an additional 5 days. the solution gelled and the gel was cured and the solution gelled within 25 minutes at room temperature. The solution was heated at 60° C. overnight. After cooling to room temperature, the gel was broken into small pieces, suspended in 2 L of deionized water, stirred and filtered. The resulting filtered material was suspended in 2 L of deionized water, stirred, the suspension was adjusted to pH 11.6 using a 50% aqueous solution of NaOH and filtered. The filtered material was washed twice with 2 L of deionized water and filtered. The filtered material was dried in a forced air oven at 60° C. to afford 10.0 g of the desired product having an in-process-swelling ratio of 136.5 ml/g.

### Example 72

### Synthesis of Compound LII

A portion of Compound XLIII was suspended in 2 L of deionized water with stirring, pH adjusted to 11.5 using a 50% aqueous solution of NaOH and filtered. The collected material was suspended in 2 L of deionized water with stirring and filtered. The filtered material was suspended in 2 L of deionized water with stirring, pH adjusted to 9.7 using a 50% aqueous solution of NaOH and filtered. The filtered material was suspended in 2 L of deionized water, stirred and filtered. The collected material was dried in a forced air oven at 60° C. to yield 7.52 g of the desired product having an in-process-swelling ratio of 10.6 g/ml.

### Example 73

### Synthesis of Compound LIII

A solution of 5 g of tris(3-chloropropyl)amine hydrochloride, 11.2 ml of tris(3-aminopropyl)amine and 7 ml of deionized water was heated under a nitrogen atmosphere at 75° C. for 3 days. Methanol was added and the solution was concentrated in vacuo using a rotary evaporator. After diluting with deionized water, the solution was dialyzed (MWCO 3500) against deionized water, concentrated in a 60° C. forced air oven and lyophilized to afford 5.38 g of the desired product having a weight-averaged molecular weight of 36,000.

### Example 74

### Synthesis of Compound LIV

A solution of 30 g of tris(3-chloropropyl)amine hydrochloride, 68 ml of tris(3-aminopropyl)amine and 40 ml of deionized water was heated under a nitrogen atmosphere at 75° C. for 3 days. Methanol was added to the solution and the solution was concentrated in vacuo using a rotary evaporator. The resulting material was dissolved into a small amount of methanol and precipitated into 2 L of diethyl ether. The solution was allowed to settle, the solvent layer was decanted, 98 ml of deionized water was added and the solution was concentrated on a rotary evaporator to 182.4 g. 14.4 ml of epichlorohydrin was added to the resulting solution and the solution gelled within 10 minutes at room temperature. The solution was cured overnight at room temperature and was heated at 60° C. for 24 hours. After cooling to room temperature, the gel was broken into small pieces, suspended in 4 L of deionized water, stirred, filtered, resuspended in 4 L of deionized water and stirred. The suspension was pH adjusted to pH 12.4 with a 50% aqueous solution of NaOH and filtered. The resulting filtered material was washed twice with 4 L of deionized water. The resulting material was suspended in 4 L of deionized water, the suspension was pH adjusted to pH 10.1 and filtered to afford material having a wet weight of 409.2 g. 204.6 g of the wet filtered material was dried in a forced air oven at 60° C. to afford 36.45 g of the desired product having an in-process-swelling ratio of 4.6 ml/g.

### Example 75

### Synthesis of Compound LV

204.6 g of the wet filtered material from Example 74 was diluted with 1.5 L of deionized water and the suspension was pH adjusted to pH 10.4 with a 50% aqueous NaOH solution. Carbon dioxide was bubbled through the solution until the suspension had a pH of 8. The resulting material was filtered and dried in a forced air oven at 60° C. to afford 39.5 g of the desired product having an in-process-swelling ratio of 4.7 ml/g.

### Example 76

### Synthesis of Compound LVI

A solution of 5 g of tris(2-chloroethyl)amine hydrochloride, 8.5 ml of tris(3-aminopropyl)amine and 6 ml of deionized water was heated under a nitrogen atmosphere at 75° C. for 3 days. Methanol was added and the solution was concentrated in vacuo using a rotary evaporator. After diluting with deionized water, the solution was dialyzed (MWCO 3500) against deionized water, concentrated in a 60° C. forced air oven and lyophilized to afford 5.38 g of the desired product having a weight-averaged molecular weight of 15,000.

### Example 77

### Synthesis of Compound LVII

A solution of 10.07 g of tris(2-chloroethyl)amine hydrochloride, 12 ml of dipropylenetriamine and 10 ml of deionized water was heated under a nitrogen atmosphere at 75° C. for 3 days. Methanol was added to the solution and the solution was concentrated in vacuo using a rotary evaporator. The resulting material was dissolved into a small amount of methanol and precipitated into 1 L of diethyl ether. The solution was allowed to settle, the solvent layer was decanted and the residue was dried in a vacuum oven at 30° C. 22 g of deionized water was added to the dried residue and 3.3 ml of epichlorohydrin was added to the resulting solution. After stirring overnight at room temperature, 1.1 ml of epichlorohydrin was added and the solution was stirred at room temperature overnight followed by heating at 60° C. overnight. A gel formed. After cooling to room temperature, the gel was broken into small pieces, suspended in 1 L of deionized water, stirred and filtered. The filtered material was washed again with 1 L of deionized water. The resulting filtered material was suspended in 1 L of deionized water, stirred, the suspension was adjusted to pH 12.3 using a 50% aqueous solution of NaOH and filtered. The filtered material was washed twice with 1 L of deionized water and filtered. The filtered material was suspended in 1 L of deionized water, stirred and the suspension was pH adjusted to 10.2 using a 50% aqueous solution of NaOH. Carbon dioxide was bubbled through the suspension until the pH of the suspension was 8. The resulting material was dried in a forced air oven at 60° C. to afford 13.98 g of the desired product having an in-process-swelling ratio of 5.7 ml/g.

### Example 78

### Synthesis of Compound LVIII

A solution of 10.09 g of tris(2-chloroethyl)amine hydrochloride, 18 ml of dipropylenetriamine and 12 ml of deionized water was heated under a nitrogen atmosphere at 75° C. for 3 days. Methanol was added to the solution and the solution was concentrated in vacuo using a rotary evaporator. The resulting material was dissolved into a small amount of methanol and precipitated into 1 L of diethyl ether. The solution was allowed to settle, the solvent layer was decanted and the residue was dried in a vacuum oven at 30° C. 28 ml of deionized water was added to the dried residue and 4.2 ml of epichlorohydrin was added to the resulting solution. After stirring overnight at room temperature, 1.8 ml of epichlorohydrin was added and the solution was stirred at room temperature overnight followed by heating at 60° C. overnight. A gel formed. After cooling to room temperature, the gel was broken into small pieces, suspended in 1 L of deionized water, stirred and filtered. The filtered material was washed again with 1 L of deionized water. The resulting filtered material was suspended in 1 L of deionized water, stirred, the suspension was adjusted to pH 12.4 using a 50% aqueous solution of NaOH and filtered. The filtered material was washed twice with 1 L of deionized water and filtered. The filtered material was suspended in 1 L of deionized water, stirred and the suspension was pH adjusted to 11.1 using a 50% aqueous solution of NaOH and filtered. The filtered material was suspended in 1 L of deionized water, stirred and the suspension was pH adjusted to 10.4 using a 50% aqueous solution of NaOH. Carbon dioxide was bubbled through the suspension until the pH of the suspension was 7.9. The resulting material was dried in a forced air oven at 60° C. to afford 16.66 g of the desired product having an in-process-swelling ratio of 12.8 ml/g.

### Example 79

### Synthesis of Compound LIX

A solution of 9.11 g of tris(3-chloropropyl)amine, 21 ml of tris(3-aminopropyl)amine and 12 ml of deionized water was heated under a nitrogen atmosphere at 75° C. for 3 days. Methanol was added and the solution was concentrated in vacuo using a rotary evaporator. The resulting material was dissolved into a small amount of methanol and precipitated into 1 L of diethyl ether. The solution was allowed to settle, the solvent layer was decanted and the residue was dried in a vacuum oven at 30° C. A 3.0 g portion of the residue was reserved. After diluting with deionized water, the remaining residue was dialyzed (MWCO 3500) against deionized water, concentrated in a 60° C. forced air oven and lyophilized to afford 9.36 g of the desired product having a weight-averaged molecular weight of 27,000 and a polydispersity of 1.4.

### Example 80

### Acidification of Compound LIV

5.0 g of Compound LIV was suspended in 500 ml of deionized water and stirred. Concentrated HCl was added to the solution until the solution had a pH of 2.0. The mixture was filtered and the collected solid was dried in a forced air oven at 60° C. to yield 7.5 g of the desired product having an in-process-swelling ratio of 5.5 ml/g.

### Example 81

### Synthesis of Compound LX

1.7 ml of epichlorohydrin was added to a stirred solution 11.74 g of Compound LII, 8.17 g of Compound LIX in 17.3 g of deionized water. A gel formed within 52 minutes and was cured at room temperature for 4 days. The gel was broken into small pieces, suspended in 1 L of deionized water, stirred and filtered. The filtered material was washed twice more with 1 L of deionized water and filtered. The resulting filtered material was suspended in 1 L of deionized water, stirred, the suspension was adjusted to pH 13 using a 50% aqueous solution of NaOH and filtered. The filtered material was washed three additional times with 1 L of deionized water and filtered. The filtered material was suspended in 1 L of deionized water, stirred and the suspension was pH adjusted to 10 using a 50% aqueous solution of NaOH. Carbon dioxide was bubbled through the suspension until the pH of the suspension was 7.7. The resulting material was dried in a forced air oven at 60° C. to afford 10.86 g of the desired product having an in-process-swelling ratio of 3.5 ml/g.

### Example 82

### Synthesis of Compound LXI

A flask was charged with 15.06 ml of divinyl sulfone, 15.52 ml of N-methyl-1,3 propane diamine and 60 ml of chloroform. The mixture exothermed to 59° C., and then was heated at 40° C. for 96 hours with stirring. The mixture was cooled to room temperature and poured into a solution of 2.5 L of methanol and 50 ml of concentrated HCl. The precipitate was collected by filtration, suspended in a hot solution of 50% (v/v) methanol and acetone, stirred for 10 min and filtered. The precipitate was re-suspended in a hot solution of 50% (v/v) methanol and acetone, stirred for 10 min and filtered. The resulting material was dried in a vacuum oven at 70° C. with a small bleed of nitrogen gas to yield 30.20 g of the desired product.

### Examples 83

### Synthesis of Compounds LXII-LXIV

Using the procedure described for Example 82, Compounds LXII-LXIV were synthesized as indicated in Table III:

TABLE III
Synthesis of Compounds LXII-LXIV

| Compound | | Multifunctional Amine- | | |
|---|---|---|---|---|
| Synthesized | Amine (amount) | Reactive Compound (amount) | Solvent (amount) | Yield (g) |
| LXII | N-methyl-1,3-propane | Divinyl sulfone (15.06 ml) | Chloroform (60 ml) | 31.36 |
| | diamine (15.52 ml) | | | |
| LXIII | 4-(aminomethyl)- | Divinyl sulfone (15.06 ml) | Chloroform (60 ml) | 33.86 |
| | piperidine (17.99 ml) | | | |
| LXIV | 1-(2-aminoethyl)- | Divinyl sulfone (15.06 ml) | Chloroform (60 ml) | 37.83 |
| | piperazine (19.68 ml) | | | |

### Example 84

### Reaction of Compound LXI with Epichlorohydrin

8.0 g of a 50% aqueous solution of NaOH was added to a solution of 15 g of Compound LXI in 15 g of deionized water. The resulting solution had a pH of 10.29. 0.569 ml of epichlorohydrin was added to this solution, stirred overnight at room temperature, and then heated to 60° C. for 1 hour. No gel was observed. A second portion of 0.569 ml of epichlorohydrin was added and the solution was heated in a closed container at 60° C. overnight. The resulting gel was broken into small pieces suspended in 2 L of deionized water, stirred for 20 min and filtered. The filtered material was re-suspended in 2 L of deionized water, stirred for 20 min and filtered. Prior to filtering the suspension had a conductivity of 70.3 us/cm. The filtered material, having a wet weight of 22.07 g was dried in a forced air oven at 60° C. to afford 2.68 g of rubbery material. The rubbery material was suspended in deionized water and the pH was adjusted with HCl to 1. The material was filtered and dried in a forced air oven at 60° C. to afford 3.51 g of the desired product.

### Example 85

### Dialysis of Compound LXI

A solution of 10 g of Compound LXI in 50 ml of deionized water was dialyzed against deionized water using MWCO 3500 tubing, until the conductivity was 17.9 uS/cm. The dialyzed material was lyophilized to afford 2.27 g.

### Example 86

### Reaction of Compound LXIV with Epichlorohydrin

9.1 g of a 50% aqueous solution of NaOH was added to a pH 3.2 solution of 15 g of Compound LXIV in 30 g of deionized water. The resulting solution had a pH of 10.51.

0.474 ml of epichlorohydrin was added to this solution and stirred overnight at room temperature. No gel was observed. A second portion of 0.474 ml of epichlorohydrin was added and the solution was stirred at room temperature for 6 hours and heated in a closed container at 60° C. for 2 hours. No gel was observed. A third portion of 0.474 ml of epichlorohydrin was added and the solution was stirred overnight at room temperature and heated in a closed container at 60° C. for 5 hours. No gel was observed. A fourth portion of 0.474 ml of epichlorohydrin was added and the solution was stirred overnight at room temperature. No gel was observed. A fifth portion of 0.474 ml of epichlorohydrin was added and the solution was heated in a closed container at 60° C. overnight. The resulting gel was broken into small pieces suspended in 2 L of deionized water, stirred for 20 min and filtered. The filtered material was re-suspended in 2 L of deionized water, stirred for 20 min and filtered. The filtered material was suspended in 2 L of deionized water and had the suspension had a conductivity of 0.31 mS/cm and a pH of 6.2. 55.3 ml of concentrated HCL was added to the suspension to adjust the pH to 1.03. The suspension was filtered and the filtered material, having a wet weight of 127.39 g, was dried in a forced-air oven at 60° C. to afford 10.83 g of the desired product having an In-Process Swelling Ratio of 10.76 ml/g.

### Test Methods

### Non-Competitive Phosphate Binding Capacity

### Buffer Preparation:

0.680 g of KH₂PO₄, 10.662 g of morpholinoethane sulfonic acid and 2.338 g of NaCl may be weighed into a 500 ml volumetric flask. 300 ml of deionized water and the solids may be dissolved. Additional deionized water may be added until the total volume of buffer is 500 ml. The pH is adjusted to 5.8 using 1 N NaOH.

### Sample Preparation:

The percent loss on drying (% LOD) by Thermogravimetric Analyzer (TGA) of 25 mg of each polymer may be determined on a Thermogravimetric Analyzer, TA Instruments, Model TGA Q 500, purged with nitrogen and using platinum pans. The following heating conditions may be used:

Heating rate: 10° C./min

End temperature: 85° C.

Hold time: 60 minutes

The % LOD may be determined as the % weight loss over 65 minutes and the result used to calculate the target sample weight with the following formula:

Weight=33.35mg/(1−(LOD/100))

### Binding Procedure:

The calculated target sample weight per polymer is weighed into each of two 50 ml plastic sample bottles. A 25 ml aliquot of the 10 mM Phosphate Buffer Solution is transferred into each of the sample bottles. The solutions are mixed well by vortexing and then shaken in an orbital shaker at 37° C. and 250 RPMs for 60 minutes. During shaking it should be ensured that the polymer particles do not adhere to the walls or lid of the sample bottle. After 60 minutes the shaker is stopped and the polymer is allowed to settle. An aliquot of exactly 2.0 ml is taken from each solution. The aliquots are filtered into small vials using a disposable syringe and 25 mm syringe filter and then diluted at a ratio of 1 part solution to 9 parts DI water. The sample bottles are shaken for a further 4 hours (total of 5 hours altogether) and the sampling procedure is repeated. Four phosphate standards are prepared by diluting the 10 mM Phosphate Buffer Solution as follows:

| | Volume of 10 mM | | |
|---|---|---|---|
| | Phosphate Solution, | Volume of H₂O, | Total Volume, |
| Std Conc, mM | mL | mL | mL |
| 0.30 | 0.75 | 24.25 | 25 |
| 0.50 | 0.50 | 9.50 | 10 |
| 0.75 | 0.75 | 9.25 | 10 |
| 1.00 | 1.00 | 9.00 | 10 |

The standards and samples are analyzed by ion chromatography using a Dionex ICS3000 instrument with conductivity detection. The 0.75 mM Standard is used as a check standard to verify the system suitability by re-injecting this standard after every 6 sample injections. The following instrument conditions are used:

Column: Dionex, AS11-HC, 4×250 mm,

Guard Column: AG11-HC, 4×50 mm,

Mobile Phase=40 mM KOH (using eluent generator)

Conductivity detector current set at 200 mA

Column Temperature: 35° C.

Flow rate: 1.5 mL/min

Injection volume: 25 μL

Run time: 6 minutes

Retention time of phosphate: ˜4 mins

A standard curve is prepared and the unbound phosphate (mM) for each test solution is calculated taking into account the 10-fold dilution. The bound phosphate is determined using the following equation:

Bound PO₄ (mmol/g)=[(10−Unbound PO₄)×Vol.×1000]/MassP

where:
Vol.=volume of test solution (L)
MassP=LOD adjusted mass of polymer (mg)
   The results from the duplicate analyses may be averaged.

### Competitive Phosphate Binding Capacity

### Buffer Preparation:

0.680 g of KH₂PO₄, 10.662 g of morpholinoethane sulfonic acid and 2.338 g of NaCl are weighed into a 500 ml volumetric flask. 300 ml of deionized water and the solids are dissolved. Additional deionized water is added until the total volume of buffer is 500 ml. A 10 mL aliquot of this solution is taken and stored for use in the preparation of standards. 3.537 g of Glycochenodeoxycholic acid, sodium salt (“GCDC”) and 2.285 g of oleic acid, sodium salt are added to the remaining 490 ml of buffer solution and the pH was adjusted to pH 5.8 with 1 N NaOH. The solution was well mixed. (Note that oleic acid does not dissolve but forms a suspension. It is ensured that the solution is well mixed and the suspended oleic acid is mixed as homogenously as possible before taking aliquots.)

### Sample Preparation:

The % LOD drying is determine as set forth above.

### Binding Procedure:

The procedure as set forth above is repeated with a 25 ml aliquot of the 10 mM Phosphate Buffer Solution with Acids.

### Determination of Particle Size and Distribution

Particle size and distribution of particle sizes may be determined as vol. % using a Malvern Mastersizer 2000 equipped with a Scirocco 2000 dry powder dispensing unit. The Mastersizer is modified by removing the ball bearings and mesh basket positioned above the venturi from the feed tray and the sample is fed to the machine and the particle size and distribution are determined using the following parameters:

Measurement time: 20 sec

Measurement snaps: 20,000

Background measurement time: 15 sec

Background measurement snaps: 15,000

Obscuration limits: 0.1 to 6%

Feeding rate: 30%

Dispersion Air pressure: 1 bar

### Determination of Mean Gray Value Using Bright Field Microscopy

After sieving to a mesh size that is −20/+50, a representative sample of the crosslinked polyamine particles may be sieved using a 35 mesh sieve. A representative sample of the particles retained on the sieve is spread over a glass slide. Images having 15-40 particles within the field of view are taken with an Olympus SZX12 Stereomicroscope equipped with an Olympus QColor 5 digital camera and set with the following parameters: 0.5× objective lens, 10× total magnification, bright field setting, and open light filters (FR, LBD and ND25).

Mean Gray Value is determined using Microsuite Biological Suite 2.3 (Build 1121). Image magnification is set at 10× using software calibration. The images are converted from the full color to 8-bit format with 230 colors. Two color phases are used: Phase I (green for the background) is set from color value 0-112, and Phase II (red for the particles) is set from color value 114-250). The minimum particle size used in the analysis is set at 1000 pixels and the fill holes option is selected. A gray value for each pixel in every particle in the image is assigned, and a mean individual particle gray value is calculated, by the software. The mean gray value, which represents the arithmetic mean of the individual particles gray value means, is determined for the imaged collection of particles. Two additional representative samples of the particles retained on the 35 mesh sieve are analyzed and the mean gray values for each of the three images are averaged to establish the Mean Gray Value.

### Bile Acid Binding Capacity

After analyzing the competitive phosphate binding of a polymer sample by ion chromatography the bile acid binding capacity of the same samples may be analyzed using HPLC according to the following procedure:

### Standard Preparation

0.177 g of GCDC is weighed into a 25 ml volumetric flask and diluted to the mark using a 100 mM morpholinoethane sulfonic acid stock solution to form a 15 mM GCDC stock solution. Four standards having the following concentrations are prepared by diluting the GCDC stock solution in volumetric flasks as follows:

| Standard Conc | Volume 15 mM | Vol. Flask |
|---|---|---|
| (mM) | GCDC stock (μL) | (mL) |
| 1.50 | 1000 | 10 |
| 1.00 | 750 | 10 |
| 0.75 | 500 | 10 |
| 0.48 | 800 | 25 |

A blank is prepared by diluting the MES buffer stock 1-to-10.

For the HPLC determination, the following parameters are used:
Column: Platinum EPS-C18, 33×7 mm, 3 micron, rocket format
MP: A=15 mM ammonium acetate, pH 5.30 (adjust pH with an 8/2 by volume acetic acid/acetonitrile solution)
MP: B=acetonitrile
Flow rate: 2 ml/min
Column Temp: 30° C.
Injection Volume: 10 μl
UV Detection: 210 nm
   and using the following gradient:

| Time (minutes) | % B |
|---|---|
| 0 | 20 |
| 2 | 20 |
| 4 | 95 |

with stop run=4.0 minutes and post run=2.5 minutes.

The following injection format may be used: Blank twice, Standards twice, Blank, then test samples once each with the 1.0 mM standard injected after every 9 sample injections for system suitability testing. The system is suitable if the difference between the original standards and the suitability standard is less than 5%.

A standard curve is set up and the unbound GCDC (mM) for each test solution is calculated. The bound GCDC is determined using the following equation:

Bound GCDC (mmol/g)=[(15−Unbound GCDC)×Vol.×1000]/MassP

where:
Vol.=volume of test solution (L) and
MassP=LOD adjusted mass of polymer (mg)

### Crosslinked Amine Polymer Urinary Phosphorous Reduction (In Vivo-Rats)

House male Sprague Dawley (SD) rats may be used for the experiments. The rats are placed singly in wire-bottom cages, fed with Purina 5002 diet, and allowed to acclimate for at least 5 days prior to experimental use.

To establish baseline phosphorus excretion, the rats are placed in metabolic cages for 48 hours. Their urine is collected and its phosphorus content analyzed with a Hitachi analyzer to determine phosphorus excretion in mg/day. Any rats with outlying values are excluded; and the remainder of the rats is distributed into groups.

Purina 5002 is used as the standard diet. The crosslinked polyamine particles being tested in each group are mixed with Purina 5002 to result in the desired final crosslinked polyamine concentration for each group. Cellulose at 0.5% by weight is used as a negative control. For each rat, 200 g of diet is prepared.

Each rat is weighed and placed on the standard diet. After 4 days the standard diet is replaced with the treatment diet (or control diet for the control group). On days 5 and 6, urine samples from the rats at 24 hours (+/−30 minutes) are collected and analyzed. The test rats are again weighed, and any weight loss or gain is calculated. Any remaining food is also weighed to calculate the amount of food consumed per day. A change in phosphorus excretion relative to cellulose negative control is calculated. Percentage reduction of urinary phosphorous is determined using the following equation:

% Reduction of Urinary Phosphorous=[(urinary phosphorous of negative control (mg/day)−urinary phosphorous of experimental (mg/day))/urinary phosphorous of negative control (mg/day)]×100.

### Crosslinked Amine Polymer Fecal Bile Acid Increase (In Vivo-Rats)

House male Sprague Dawley (SD) rats may be used for the experiments. The rats are placed singly in wire-bottom cages, fed with Purina 5002 diet, and allowed to acclimate for at least 5 days prior to experimental use.

After acclimatization, the rats are split into test groups with 6 rats per group. Purina 5002 with NaH₂PO₄ at a concentration of 0.4 wt % phosphate added is used as the standard diet. The crosslinked polyamine being tested in each group is mixed with the standard diet to result in the desired final crosslinked polyamine concentration for each group. Cellulose at 4.0% by weight is used as a negative control.

Each rat is weighed and placed on its respective treatment diet. On day six, the rats are placed in metabolism cages specifically designed to separate and collect fecal material for 24 hours. The fecal material is collected, freeze dried, weighed and ground into a powder. 500 mgs of the powder is added to an extraction vessel and heated to 100° C. at 1500 psi for 10 minutes in an extraction solvent consisting of 80% methanol/20% 500 mM KOH. 250 μls of the extract is evaporated in a speed vac at 45° C. for 2 hours and then is reconstituted in a 50% mixture of calf serum and saline. The bile acid concentration may be quantitated using a Total Bile Acids colorometric assay available from Diazyme Laboratories, Inc. at catalog number DZ092A.

A change in fecal bile acid excretion relative to the cellulose negative control is calculated. Percentage increase of fecal bile acid was determined using the following equation:

% Increase in Fecal Bile Acid=[(Fecal Bile Acid of experimental (mg/day)−Fecal Bile Acid of negative control (mg/day))/Fecal Bile Acid of negative control (mg/day)]×100.

In-Process Swelling Ratio (ml/g)

The in-process swelling ratio (SR) of polymers may be determined by the following equation:

SR=(weight of wet gel (g)−weight of dry polymer (g))/weight of dry polymer (g).

All publications and patent applications mentioned in this specification are herein incorporated by reference to the same extent as if each individual publication or patent application was specifically and individually indicated to be incorporated by reference.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives and equivalents to the embodiments of the invention described herein may be employed in practicing the invention. It is intended that the following claims define the scope of the invention and that methods and structures within the scope of these claims and their equivalents be covered thereby.

1. A pharmaceutical composition comprising crosslinked polyamine particles, or pharmaceutically acceptable salts thereof, having a particle size distribution wherein greater than 10 vol. % of the particles have a particle size greater than 500 μm.

2. The pharmaceutical composition according to claim 1, wherein said particles have a d₅₀ between 675 μm and 1000 μm.

3. The pharmaceutical composition according to claim 2, wherein said particles have a distribution such that the d₁₀ value is between 350 μm and 650 μm and/or the d₉₀ value is between 1100 μm and 1400 μm.

4. A pharmaceutical composition comprising:
crosslinked polyamine particles, or a pharmaceutically acceptable salt thereof, said particles having a mean gray value of greater than 180.

5. The pharmaceutical composition according to claim 4, wherein said particles have a mean gray value of between 190 and 230.

6. The pharmaceutical composition according to claim 5, wherein said particles have a d₅₀ between 675 μm and 1000 μm.

7. The pharmaceutical composition according to claim 6, wherein said particles have a distribution such that the d₁₀ value is between 350 μm and 650 μm and/or the d₉₀ value is between 1100 μm and 1400 μm.

8. A pharmaceutical composition comprising:
aggregate particles comprising constituent particles comprising crosslinked polyamine.

9. The pharmaceutical composition according to claim 8, wherein said aggregate particles comprise from 500 to 1000 of said constituent particles.

10. The pharmaceutical composition according to claim 9, wherein said constituent particles have a d₅₀ between 70 and 120 μm.

11. The pharmaceutical composition according to claim 10, wherein said aggregate particles are formed by aggregating 2 or more constituent particles comprising crosslinked polyamine.

12. The pharmaceutical composition of claim 11, wherein said aggregating comprises hydrating said constituent particles.

13. The pharmaceutical composition according to claim 12, wherein said aggregate particles have a d₅₀ between 675 μm and 1000 μm.

14. The pharmaceutical composition according to claim 13, wherein said aggregate particles have a distribution such that the d₁₀ value is between 350 μm and 650 μm and/or the d₉₀ value is between 1100 μm and 1400 μm.

15. The pharmaceutical composition according to claim 14, wherein said particles have a particle size distribution wherein greater than 50 vol. % of the particles have a particle size between 500 μm and 1500 μm.

16. The pharmaceutical composition according to claim 15, wherein said crosslinked polyamine is at least partially protonated with carbonate, bicarbonate or a mixture thereof as the counterion.

17. The pharmaceutical composition according to claim 16, wherein said crosslinked polyamine particles are crosslinked with epichlorohydrin.

18. The pharmaceutical composition according to claim 17, wherein said crosslinked polyamine particles comprise crosslinked dendrimers.

19. The pharmaceutical composition according to claim 18, wherein said crosslinked polyamine particles comprise crosslinked hyperbranched polymers or crosslinked hyperbranched copolymers.

20. The pharmaceutical composition according to claim 19, further comprising a pharmaceutically acceptable excipient.
